(19) United States
(12) Patent Application Publication
(10) Pub. No.: US 2023/0348468 A1
(43) Pub. Date: Nov. 2, 2023

(54) 1H-PYRAZOLO[4,3-d]PYRIMIDINE COMPOUNDS AS TOLL-LIKE RECEPTOR 7 (TLR7) AGONISTS

(71) Applicant: BRISTOL-MYERS SQUIBB COMPANY, Princeton, NJ (US)

(72) Inventors: Matthew COX, San Francisco, CA (US); Naidu S. CHOWDARI, Dublin, CA (US); Ashvinikumar V. GAVAI, Princeton Junction, NJ (US); Sanjeev GANGWAR, Foster City, CA (US); Christine M. TARBY, Lawrenceville, NJ (US); Yam B. POUDEL, Fremont, CA (US); Murugaiah ANDAPPAN MURUGAIAH SUBBAIAH, Bommasandra Bangalore (IN)

(21) Appl. No.: 17/793,278

(22) PCT Filed: Jan. 26, 2021
(86) PCT No.: PCT/US2021/014980

§ 371 Date: Jul. 15, 2022

Related U.S. Application Data
(60) Provisional application No. 63/058,230, filed on Jul. 29, 2020.
(60) Provisional application No. 62/966,144, filed on Jan. 27, 2020.

(51) Int. Cl. C07D 487/04; C07K 16/28

(52) U.S. Cl. CPC C07D 487/04; C07K 16/2818; C07K 16/2827

(57) ABSTRACT

Compounds according to formula I or II are useful as agonists of Toll-like receptor 7 (TLR7). (I) (II) Such compounds can be used in cancer treatment, especially in combination with an anti-cancer immunotherapy agent, or as a vaccine adjuvant.

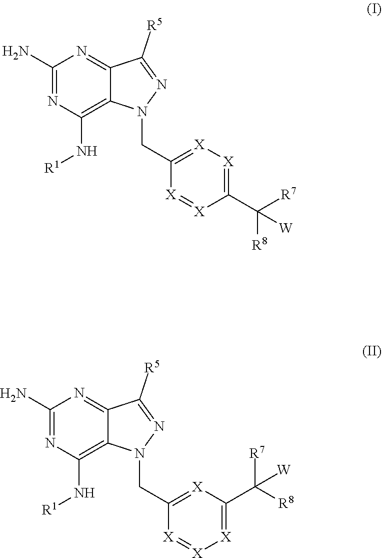

### CROSS-REFERENCE TO RELATED APPLICATIONS

This application claims the benefit under 35 U.S.C. § 119(e) of U.S. Provisional Application Ser. No. 63/058,230, filed Jul. 29, 2020, and U.S. Provisional Application Ser. No. 62/966,144, filed Jan. 27, 2020; the disclosures of which are incorporated herein by reference.

### BACKGROUND OF THE DISCLOSURE

This disclosure relates to Toll-like receptor 7 (“TLR7”) agonists and conjugates thereof, and methods for the preparation and use of such agonists and their conjugates.

Toll-like receptors (“TLRs”) are receptors that recognize pathogen-associated molecular patterns (“PAMPs”), which are small molecular motifs conserved in certain classes of pathogens. TLRs can be located either on a cell's surface or intracellularly. Activation of a TLR by the binding of its cognate PAMP signals the presence of the associated pathogen inside the host—i.e., an infection—and stimulates the host's immune system to fight the infection. Humans have 10 TLRs, named TLR1, TLR2, TLR3, and so on.

The activation of a TLR—with TLR7 being the most studied—by an agonist can have a positive effect on the action of vaccines and immunotherapy agents in treating a variety of conditions other than actual pathogen infection, by stimulating the immune response overall. Thus, there is considerable interest in the use of TLR7 agonists as vaccine adjuvants or as enhancers in cancer immunotherapy. See, for example, Vasilakos and Tomai 2013, Sato-Kaneko et al. 2017, Smits et al. 2008, and Ota et al. 2019.

TLR7, an intracellular receptor located on the membrane of endosomes, recognizes PAMPs associated with single-stranded RNA viruses. Its activation induces secretion of Type I interferons such as IFNα and IFNβ (Lund et al. 2004). TLR7 has two binding sites, one for single stranded RNA ligands (Berghöfer et al. 2007) and one for small molecules such as guanosine (Zhang et al. 2016).

TLR7 can bind to, and be activated by, guanosine-like synthetic agonists such as imiquimod, resiquimod, and gardiquimod, which are based on a 1H-imidazo[4,5-c]quinoline scaffold. For a review of small-molecule TLR7 agonists, see Cortez and Va 2018.

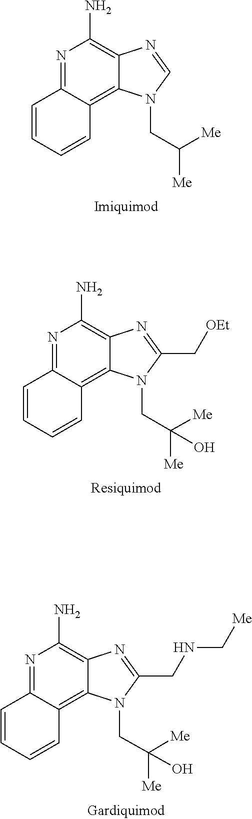 [SMILES: CC(C)Cn1cnc2c(N)nc3ccccc3c21 ; CCOCc1nc2c(N)nc3ccccc3c2n1CC(C)(C)O ; CCNCc1nc2c(N)nc3ccccc3c2n1CC(C)(C)O]

Synthetic TLR7 agonists based on a pteridinone molecular scaffold are also known, as exemplified by vesatolimod (Desai et al. 2015).

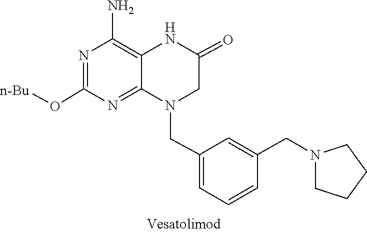

Other synthetic TLR7 agonists based on a purine-like scaffold have been disclosed, frequently according to the general formula (A):

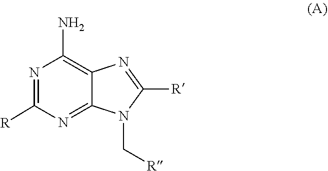

where R, R′, and R″ are structural variables, with R″ typically containing an unsubstituted or substituted aromatic or heteroaromatic ring.

Disclosures of bioactive molecules having a purine-like scaffold and their uses in treating conditions such as fibrosis, inflammatory disorders, cancer, or pathogenic infections include: Akinbobuyi et al. 2015 and 2016; Barberis et al. 2012; Carson et al. 2014; Ding et al. 2016, 2017a, and 2017b; Graupe et al. 2015; Hashimoto et al. 2009; He et al. 2019a and 2019b; Holldack et al. 2012; Isobe et al. 2009a and 2012; Poudel et al. 2019a and 2019b; Pryde 2010; and Young et al. 2019.

The group R″ can be pyridyl: Bonfanti et al. 2015a and 2015b; Halcomb et al. 2015; Hirota et al. 2000; Isobe et al. 2002, 2004, 2006, 2009a, 2009b, 2011, and 2012; Kasibhatla et al. 2007; Koga-Yamakawa et al. 2013; Musmuca et al. 2009; Nakamura 2012; Ogita et al. 2007; and Yu et al. 2013.

There are disclosures of related molecules in which the 6,5-fused ring system of formula (A)—a pyrimidine six member ring fused to an imidazole five member ring—is modified. (a) Dellaria et al. 2007, Jones et al. 2010 and 2012, and Pilatte et al. 2017 disclose compounds in which the pyrimidine ring is replaced by a pyridine ring. (b) Chen et al. 2011, Coe et al. 2017, Poudel et al. 2020a and 2020b, and Zhang et al. 2018 disclose compounds in which the imidazole ring is replaced by a pyrazole ring. (c) Cortez et al. 2017 and 2018; Li et al. 2018; and McGowan et al. 2016a, 2016b, and 2017 disclose compounds in which the imidazole ring is replaced by a pyrrole ring.

Bonfanti et al. 2015b and 2016 and Purandare et al. 2019 disclose TLR7 modulators in which the two rings of a purine moiety are spanned by a macrocycle:

A TLR7 agonist can be conjugated to a partner molecule, which can be, for example, a phospholipid, a poly(ethylene glycol) (“PEG”), an antibody, or another TLR (commonly TLR2). Exemplary disclosures include: Carson et al. 2013, 2015, and 2016, Chan et al. 2009 and 2011, Cortez et al. 2017, Gadd et al. 2015, Lioux et al. 2016, Maj et al. 2015, Vernejoul et al. 2014, and Zurawski et al. 2012. A frequent conjugation site is at the R″ group of formula (A).

Jensen et al. 2015 discloses the use of cationic lipid vehicles for the delivery of TLR7 agonists.

Some TLR7 agonists, including resiquimod are dual TLR7/TLR8 agonists. See, for example, Beesu et al. 2017, Embrechts et al. 2018, Lioux et al. 2016, and Vernejoul et al. 2014.

Full citations for the documents cited herein by first author or inventor and year are listed at the end of this specification.

### BRIEF SUMMARY OF THE DISCLOSURE

This specification relates to compounds having a 1H-pyrazolo[4,3d]pyrimidine aromatic system, having activity as TLR7 agonists.

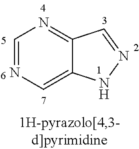

In one aspect, there is provided a compound with a structure according to formula (I) or (II)

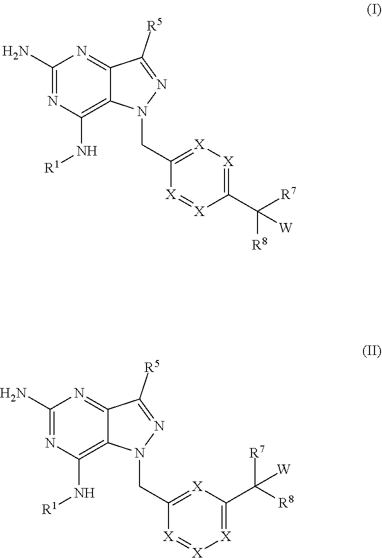

wherein
each X is independently N or CR²;
W is R³ or

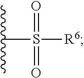

R¹ is (C₁-C₅ alkyl),
   (C₂-C₅ alkenyl),
   (C₁-C₈ alkanediyl)₀₋₁(C₃-C₆ cycloalkyl),
   (C₁-C₈ alkanediyl)₀₋₁(C₅-C₁₀ spiroalkyl),
   (C₂-C₈ alkanediyl)OH,
   (C₂-C₈ alkanediyl)₀₋₁(C₁-C₃ alkyl),
   (C₁-C₄ alkanediyl)₀₋₁(5-6 membered heteroaryl),
   (C₁-C₄ alkanediyl)₀₋₁phenyl,
   (C₁-C₄ alkanediyl)CF₃,
   (C₂-C₈ alkanediyl)N[C(═O)](C₁-C₃ alkyl),
   or
   (C₂-C₈ alkanediyl)NR^{x}R^{y};
each R² is independently H, O(C₁-C₃ alkyl), S(C₁-C₃ alkyl), SO₂(C₁-C₃ alkyl), C₁-C₃ alkyl, O(C₃-C₄ cycloalkyl), S(C₃-C₄ cycloalkyl), SO₂(C₃-C₄ cycloalkyl), C₃-C₄ cycloalkyl, Cl, F, CN; or [C(═O)]₀₋₁NR^{x}R^{y};
R³ is NH₂,
   NH[C(═O)]₀₋₁(C₁-C₅ alkyl),
   N(C₁-C₅ alkyl)₂,
   NH[C(═O)]₀₋₁(C₁-C₄ alkanediyl)₀₋₁(C₃-C₈ cycloalkyl),
   NH[C(═O)]₀₋₁(C₁-C₄ alkanediyl)₀₋₁(C₄-C₁₀ bicycloalkyl),
   NH[C(═O)]₀₋₁(C₁-C₄ alkanediyl)₀₋₁(C₅-C₁₀ spiroalkyl),
   N(C₃-C₆ cycloalkyl)₂,
   N[C₁-C₃ alkyl]C(═O)(C₁-C₆ alkyl),
   NH(SO₂)(C₁-C₅ alkyl),
   NH(SO₂)(C₁-C₄ alkanediyl)₀₋₁(C₃-C₈ cycloalkyl),
   NH(SO₂)(C₁-C₄ alkanediyl)₀₋₁(C₄-C₁₀ bicycloalkyl),
   NH(SO₂)(C₁-C₄ alkanediyl)₀₋₁(C₅-C₁₀ spiroalkyl),
   a 6-membered aromatic or heteroaromatic moiety,
   a 5-membered heteroaromatic moiety, or
   a moiety having the structure

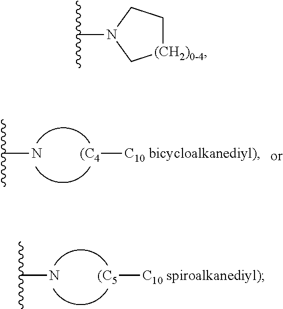

R⁵ is H, C₁-C₅ alkyl, C₂-C₅ alkenyl, C₃-C₆ cycloalkyl, halo, O(C₁-C₅ alkyl), (C₁-C₄ alkanediyl)OH, (C₁-C₄ alkanediyl)O(C₁-C₃ alkyl), phenyl, NH(C₁-C₅ alkyl), 5 or 6 membered heteroaryl,

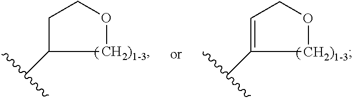

R⁶ is NH₂,
   (NH)₀₋₁(C₁-C₅ alkyl),
   N(C₁-C₈ alkyl)₂,
   (NH)₀₋₁(C₁-C₄ alkanediyl)₀₋₁(C₃-C₈ cycloalkyl),
   (NH)₀₋₁(C₁-C₄ alkanediyl)₀₋₁(C₄-C₁₀ bicycloalkyl),
   (NH)₀₋₁(C₁-C₄ alkanediyl)₀₋₁(C₅-C₁₀ spiroalkyl),
   N(C₃-C₆ cycloalkyl)₂,
   or
   a moiety having the structure

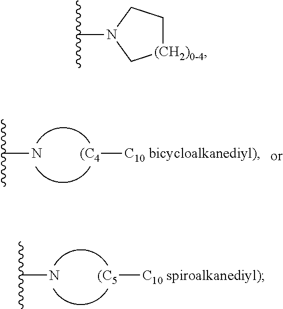

R⁷ and R⁸ are independently
   C₁-C₄ alkyl,
   C₂-C₄ alkylene,
   C₃-C₄ cycloalkyl,
   or R⁷ and R⁸ combine with the carbon to which they are bonded to form a 3- to 7-membered cycloalkyl moiety;
R^{x} and R^{y} are independently H or C₁-C₃ alkyl or R^{x} and R^{y} combine with the nitrogen to which they are bonded to form a 3- to 7-membered heterocycle;
wherein in R¹, R², R³, R⁵, R⁶, R⁷, and R⁸
   an alkyl moiety, alkanediyl moiety, cycloalkyl moiety, or a moiety of the formula

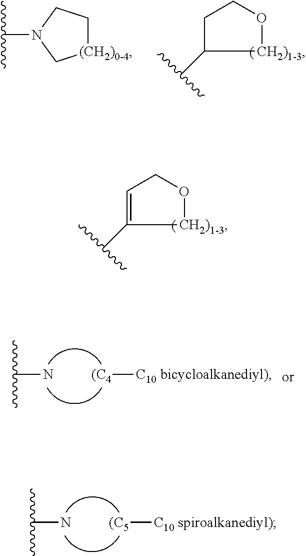

is optionally substituted with one or more substituents selected from OH, halo, CN, (C₁-C₃ alkyl), O(C₁-C₃ alkyl), C(═O)(C₁-C₃ alkyl), SO₂(C₁-C₃ alkyl), NR^{x}R^{y}, (C₁-C₄ alkanediyl)OH, (C₁-C₄ alkanediyl)O(C₁-C₃ alkyl);
and
an alkyl, alkanediyl, cycloalkyl, or moiety of the formula

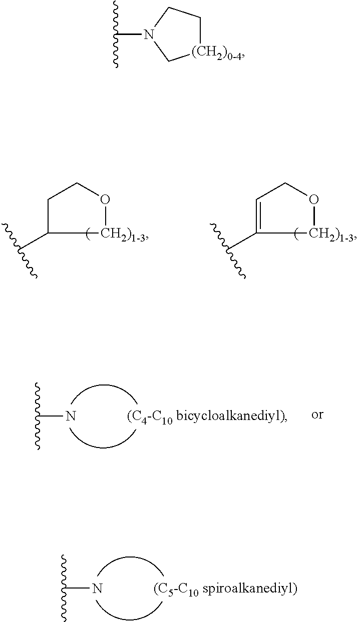

may have a CH₂ group replaced by O, SO₂, CF₂, C(═O), NH,
N[C(═O)]₀₋₁(C₁-C₃ alkyl),
N[C(═O)]₀₋₁(C₁-C₄ alkanediyl)CF₃,
N[C(═O)]₀₋₁(C₁-C₄ alkanediyl)OH,
or
N[C(═O)]₀₋₁(C₁-C₄ alkanediyl)₀₋₁(C₃-C₅ cycloalkyl).

Compounds disclosed herein have activity as TLR7 agonists and some can be conjugated to an antibody for targeted delivery to a target tissue or organ of intended action. They can also be PEGylated, to modulate their pharmaceutical properties.

Compounds disclosed herein, or their conjugates or their PEGylated derivatives, can be used in the treatment of a subject suffering from a condition amenable to treatment by activation of the immune system, by administering to such subject a therapeutically effective amount of such a compound or a conjugate thereof or a PEGylated derivative thereof, especially in combination with a vaccine or a cancer immunotherapy agent.

### DETAILED DESCRIPTION OF THE DISCLOSURE

### Compounds

In one aspect, W is R³.

In one aspect, in formula (I) the moiety

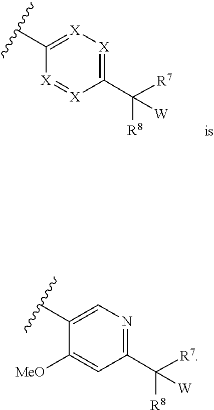

In one aspect, in formula (II) the moiety

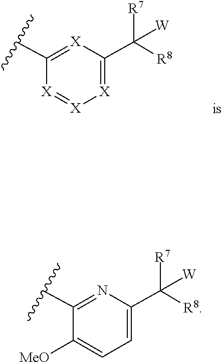

In one aspect, compounds of this disclosure are according to formula (Ia), wherein R¹, R³, R⁷ and R⁸ are as defined in respect of formula (I):

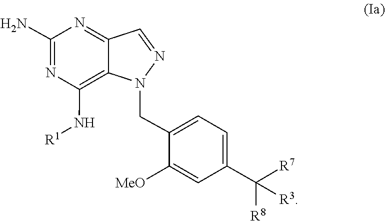

In another aspect, compounds of this disclosure are according to formula (IIa), wherein R¹, R³, R⁷ and R⁸ are as defined in respect of formula (II):

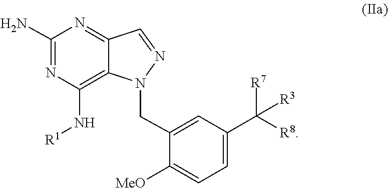

In one embodiment, each of R⁷ and R⁸ is C₁-C₄ alkyl. In such instance, R⁷ and R⁸ can be but are not necessarily the same C₁-C₄ alkyl.

In another embodiment, R⁷ and R⁸ are both Me.

In another embodiment,

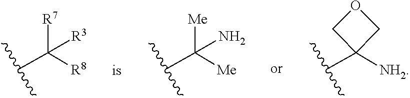

In another embodiment, R⁷ and R⁸ combine with the carbon to which they are bonded to form a 3- to 7-membered cycloakyl moiety. Optionally, such cycloalkyl moiety has a CH₂ group replace by O; preferably to form an oxetanyl ring, so that

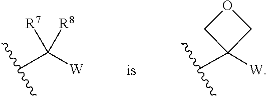

Examples of suitable groups R¹ include:

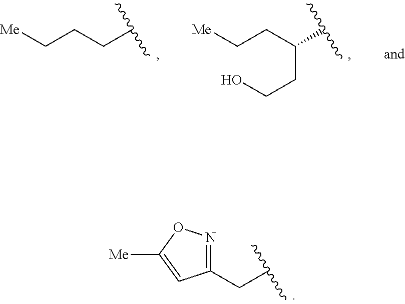 [SMILES: *CCCC ; *[C@@H](CCC)CCO ; *Cc1cc(C)on1]

R² preferably is OMe, O(cyclopropyl), or OCHF₂, more preferably OMe.

In one aspect, R⁵ is H.

In one aspect, there is provided a compound according to formula (Ia) or (IIa)

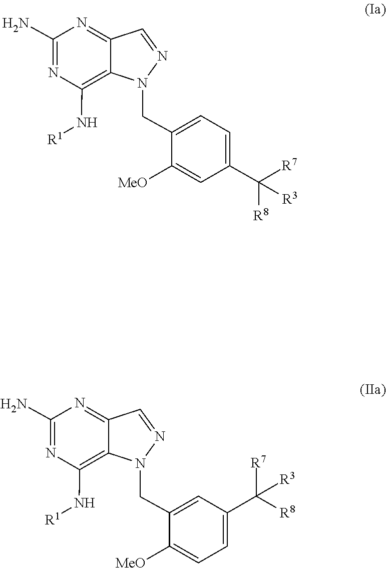

wherein
R¹ is

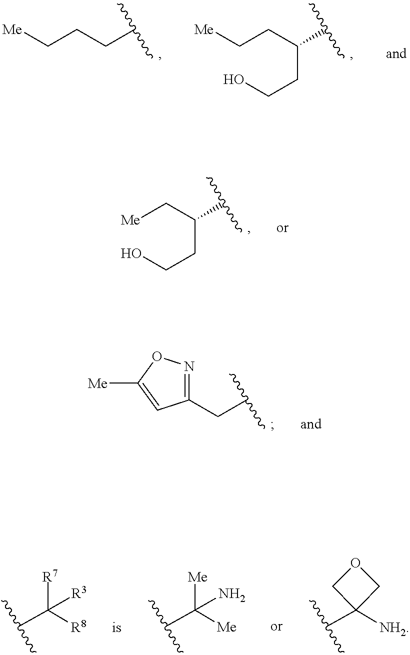

In another aspect, there is provided a compound with a structure according to formula (I′) or (II′)

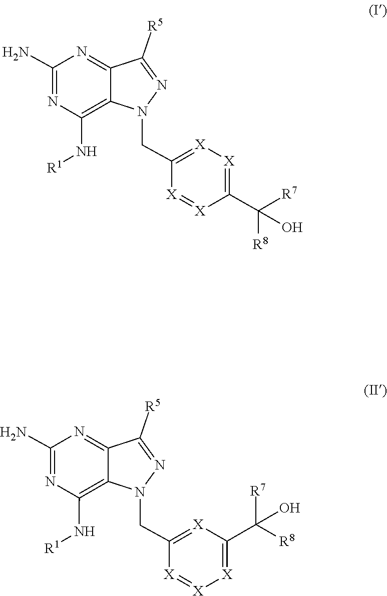

wherein
each X is independently N or CR²;
R¹ is (C₁-C₅ alkyl),
   (C₂-C₅ alkenyl),
   (C₁-C₈ alkanediyl)₀₋₁(C₃-C₆ cycloalkyl),
   (C₁-C₈ alkanediyl)₀₋₁(C₅-C₁₀ spiroalkyl),
   (C₂-C₈ alkanediyl)OH,
   (C₂-C₈ alkanediyl)O(C₁-C₃ alkyl),
   (C₁-C₄ alkanediyl)₀₋₁(5-6 membered heteroaryl),
   (C₁-C₄ alkanediyl)₀₋₁phenyl,
   (C₁-C₄ alkanediyl)CF₃,
   (C₂-C₈ alkanediyl)N[C(═O)](C₁-C₃ alkyl),
   or
   (C₂-C₈ alkanediyl)NR^{x}R^{y};
each R² is independently H, O(C₁-C₃ alkyl), S(C₁-C₃ alkyl), SO₂(C₁-C₃ alkyl), C₁-C₃ alkyl, O(C₃-C₄ cycloalkyl), S(C₃-C₄ cycloalkyl), SO₂(C₃-C₄ cycloalkyl), C₃-C₄ cycloalkyl, Cl, F, CN; or [C(═O)]₀₋₁NR^{x}R^{y};
R⁵ is H, C₁-C₅ alkyl, C₂-C₅ alkenyl, C₃-C₆ cycloalkyl, halo, O(C₁-C₅ alkyl), (C₁-C₄ alkanediyl)OH, (C₁-C₄ alkanediyl)O(C₁-C₃ alkyl), phenyl, NH(C₁-C₅ alkyl), 5 or 6 membered heteroaryl,

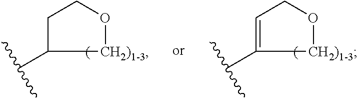

R⁷ and R⁸ are independently
   C₁-C₄ alkyl,
   C₂-C₄ alkylene,
   C₃-C₄ cycloalkyl,
   or R⁷ and R^{B} combine with the carbon to which they are bonded to form a 3- to 7-membered cycloalkyl moiety;
R^{x} and R^{y} are independently H or C₁-C₃ alkyl or R^{x} and R^{y} combine with the nitrogen to which they are bonded to form a 3- to 7-membered heterocycle;
wherein in R¹, R², R⁵, R⁷, and R⁸
an alkyl moiety, alkanediyl moiety, cycloalkyl moiety, or a moiety of the formula

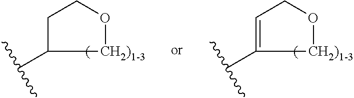

is optionally substituted with one or more substituents selected from OH, halo, CN, (C₁-C₃ alkyl), O(C₁-C₃ alkyl), C(═O)(C₁-C₃ alkyl), SO₂(C₁-C₃ alkyl), NR^{x}R^{y}, (C₁-C₄ alkanediyl)OH, (C₁-C₄ alkanediyl)O(C₁-C₃ alkyl);
and
an alkyl, alkanediyl, cycloalkyl, or moiety of the formula

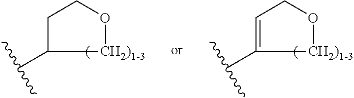

may have a CH₂ group replaced by O, SO₂, CF₂, C(═O), NH,
N[C(═O)]₀₋₁(C₁-C₃ alkyl),
N[C(═O)]₀₋₁(C₁-C₄ alkanediyl)CF₃,
N[C(═O)]₀₋₁(C₁-C₄ alkanediyl)OH,
or
N[C(═O)]₀₋₁(C₁-C₄ alkanediyl)₀₋₁(C₃-C₅ cycloalkyl).

In another aspect, there is provided a compound having a structure according to formula (Ia′)

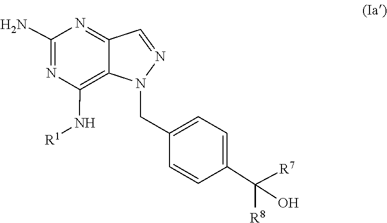

wherein
R¹ is

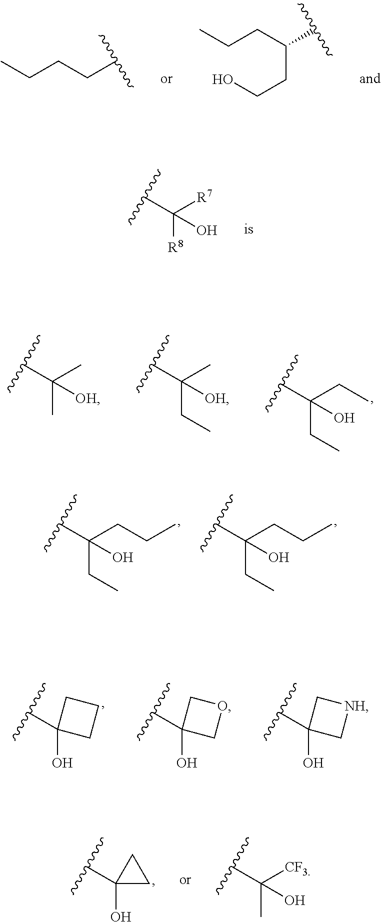

Specific examples of compounds disclosed herein are shown in the following Table A. The table also provides data relating to biological activity: human TLR7 agonism (reporter) assay and/or induction of the CD69 gene in human whole blood, determined per the procedure provided hereinbelow. The right-most column contains info analytical data (mass spectrum, HPLC retention time, and NMR). In one embodiment, a compound of this disclosure has (a) a human TLR7 (hTLR7) Reporter Assay EC₅₀ value of less than 1,000 nM and (b) a human whole blood (hWB) CD69 induction EC₅₀ value of less than 1,000 nM. (Where an assay was performed multiple times, the reported value is an average.)

TABLE A

| | | hTLR7 | hWB | Analytical Data |
|---|---|---|---|---|
| | | Agonism | CD69 | (Mass spectrum, LC/MS Retention Time, |
| Cpd | | EC₅₀ | EC₅₀ | ¹H NMR (500 MHz, DMSO-d6) unless |
| No. | Structure | (nM) | (nM) | noted otherwise) |
| 101 | 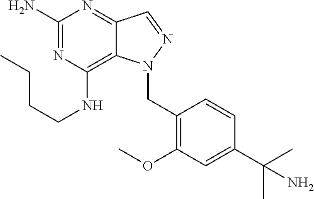 [SMILES: CCCCNc1nc(N)nc2cnn(Cc3ccc(C(C)(C)N)cc3OC)c12] | 76.6 | 11.6 | LC/MS [M + H]⁺ = 384.1 RT (min) = 1.24 (LC/MS Procedure A) δ 7.55 (s, 1H), 7.21 (s, 1H), 6.94 (br d, J = 7.9 Hz, 1H), 6.50 (br d, J = 7.9 Hz, 1H), 6.48-6.44 (m, 1H), 5.67-5.56 (m, 4H), 3.86 (s, 3H), 3.40 (m, 2H), 1.62-1.44 (m, 2H), 1.41 (s, 6H), 1.32-1.15 (m, 2H), 0.85 (t, J = 7.3 Hz, 3H) |
| | 1-(4-(2-Aminopropan-2-yl)-2- | | | |
| | methoxybenzyl)-N7-butyl-1H- | | | |
| | pyrazolo[4,3-d]pyrimidine- | | | |
| | 5,7-diamine | | | |
| 102 | 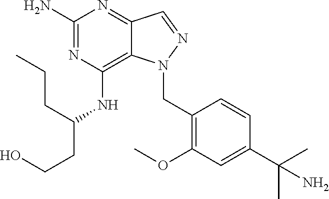 | 7.2 | 1.8 | LC/MS [M + H]⁺ = 428.1 RT (min) = 1.12 (LC/MS Procedure A) δ 7.56 (s, 1H), 7.24 (s, 1H), 7.07-6.92 (m, 1H), 6.41 (d, J = 8.0 Hz, 1H), 5.67-5.51 (m, 4H), 4.31 (br d, J = 2.9 Hz, 1H), 3.87 (s, 3H), 3.34-3.22 (m, 1H), 1.63 (br dd, J = 12.9, 5.3 Hz, 1H), 1.51-1.39 (m, 3H), 1.37 (s, 6H), 1.16-1.00 (m, 2H), 0.78 (t, J = 7.3 Hz, 3H) |
| | (S)-3-((5-Amino-1-(4-(2-aminopro- | | | |
| | pan-2-yl)-2-methoxybenzyl)-1H- | | | |
| | pyrazolo[4,3-d]pyrimidin-7- | | | |
| | yl)amino)hexan-1-ol | | | |
| 103 | 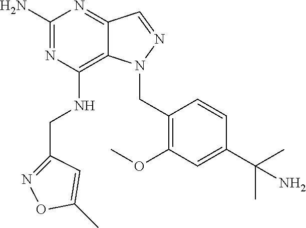 [SMILES: COc1cc(C(C)(C)N)ccc1Cn1ncc2nc(N)nc(NCc3cc(C)on3)c21] | 202.0 | | LC/MS [M + H]⁺ = 423.1 RT (min) = 1.02 (LC/MS Procedure A) δ 7.60 (s, 1H), 7.20 (s, 1H), 6.95 (br d, J = 6.4 Hz, 1H), 6.55 (d, J = 7.9 Hz, 1H), 6.08 (s, 1H), 5.76 (s, 2H), 5.62 (s, 2H), 4.67 (br d, J = 4.3 Hz, 2H), 3.79 (s, 3H), 2.36 (s, 3H), 1.41 (s, 6H). |
| | 1-(4-(2-Aminopropan-2-yl)-2- | | | |
| | methoxybenzyl)-N7-((5-methyl- | | | |
| | isoxazol-3-yl)methyl)-1H-pyrazolo | | | |
| | [4,3-d]pyrimidine-5,7-diamine | | | |
| 104 | 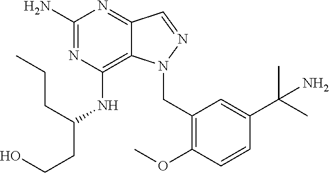 | 34.5 | 12.7 | LC/MS [M + H]⁺ = 428.2 RT (min) = 1.23 (LC/MS Procedure A) δ 7.49 (s, 1H), 7.36 (dd, J = 8.6, 2.4 Hz, 1H), 6.92 (d, J = 8.7 Hz, 1H), 6.64 (d, J = 2.5 Hz, 1H), 5.66-5.40 (m, 4H), 4.25 (m, 1H), 3.77 (s, 3H), 3.25 (m, 2H), 1.60-1.24 (m, 6H), 1.14 (s, 3H), 1.12 (s, 3H), 0.97 (m, 2H), 0.69 (t, J = 7.3 Hz, 3H) |
| | (S)-3-((5-Amino-1-(5-(2-aminopro- | | | |
| | pan-2-yl)-2-methoxybenzyl)-1H- | | | |
| | pyrazolo[4,3-d]pyrimidin-7- | | | |
| | yl)amino)hexan-1-ol | | | |
| 105 | 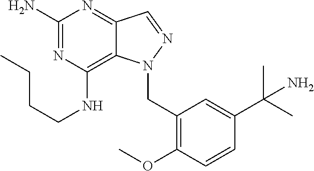 [SMILES: CCCCNc1nc(N)nc2cnn(Cc3cc(C(C)(C)N)ccc3OC)c12] | — | 50.6 | LC/MS [M + H]⁺ = 383.9 RT (min) = 1.33 (LC/MS Procedure A) δ 7.55 (s, 1H), 7.44 (d, J = 7.9 Hz, 1H), 7.07 (d, J = 8.6 Hz, 1H), 6.87 (d, J = 2.4 Hz, 1H), 6.51 (d, J = 5.8 Hz, 1H), 5.61 (s, 4H), 3.80 (s, 4H), 2.55 (s, 2H), 1.56-1.44 (m, 2H), 1.39 (s, 6H), 1.23 (q, J = 7.5 Hz, 2H), 0.86 (t, J = 7.4 Hz, 3H) |
| | 1-(5-(2-Aminopropan-2-yl)-2- | | | |
| | methoxybenzyl)-N7-butyl-1H- | | | |
| | pyrazolo[4,3-d]pyrimidine- | | | |
| | 5,7-diamine | | | |
| 106 | 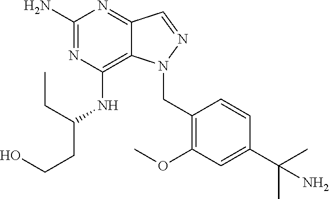 | 210.9 | 3.7 | LC/MS [M + H]⁺ = 414.2 RT (min) = 1.09 (LC/MS Procedure A) δ 7.57 (s, 1H), 7.21 (s, 1H), 6.94 (br d, J = 8.0 Hz, 1H), 6.49 (d, J = 8.0 Hz, 1H), 5.85- 5.55 (m, 4H), 4.23 (br s, 1H), 3.96-3.81 (m, 3H), 3.45-3.26 (m, 1H), 1.76-1.60 (m, 2H), 1.60-1.47 (m, 8H), 1.41 (dt, J = 14.0, 7.1 Hz, 2H), 0.76-0.58 (m, 3H) |
| | (S)-3-((5-amino-1-(4-(2-amino- | | | |
| | propan-2-yl)-2-methoxybenzyl)-1H- | | | |
| | pyrazolo[4,3-d]pyrimidin-7- | | | |
| | yl)amino)pentan-1-ol | | | |
| 107 | 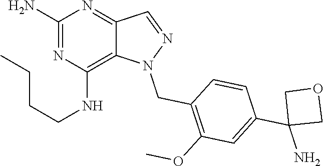 [SMILES: CCCCNc1nc(N)nc2cnn(Cc3ccc(C4(N)COC4)cc3OC)c12] | 246.7 | 42.6 | LC/MS [M + H]⁺ = 398.2 RT (min) = 1.28 (LC/MS Procedure A) δ 8.37 (br s, 1H), 7.89 (br s, 1H), 7.76 (s, 1H), 7.16 (s, 1H), 7.05 (br d, J = 8.5 Hz, 1H), 6.98 (d, J = 7.6 Hz, 1H), 5.76 (s, 2H), 4.94 (s, 4H), 3.80 (s, 2H), 3.65-3.54 (m, 1H), 1.62 (quin, J = 7.4 Hz, 2H), 1.31 (dq, J = 15.0, 7.3 Hz, 2H), 0.92 (t, J = 7.3 Hz, 3H). |
| | 1-(4-(3-aminooxetan-3-yl)-2- | | | |
| | methoxybenzyl)-N7-butyl-1H- | | | |
| | pyrazolo-[4,3-d]pyrimidine- | | | |
| | 5,7-diamine | | | |
| 108 | 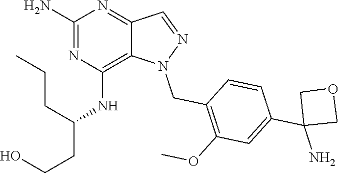 [SMILES: CCC[C@@H](CCO)Nc1nc(N)nc2cnn(Cc3ccc(C4(N)COC4)cc3OC)c12] | 43.4 | 3.3 | LC/MS [M + H]⁺ = 442.3 RT (min) = 0.97 (LC/MS Procedure A) δ 7.59 (s, 1H), 7.22 (s, 1H), 7.05 (br d, J = 7.9 Hz, 1H), 6.50 (d, J = 7.9 Hz, 1H), 5.82- 5.54 (m, 4H), 4.70 (d, J = 6.1 Hz, 2H), 4.63 (dd, J = 6.0, 2.3 Hz, 2H), 4.33 (br d, J = 7.6 Hz, 1H), 3.89 (s, 3H), 3.31 (br t, J = 6.4 Hz, 1H), 1.79-1.60 (m, 1H), 1.52 (br dd, J = 13.6, 7.8 Hz, 1H), 1.47-1.32 (m, 2H), 1.15-1.00 (m, 2H), 0.77 (t, J = 7.3 Hz, 3H) |
| | (S)-3-((5-amino-1-(4-(3-amino- | | | |
| | oxetan-3-yl)-2-methoxybenzyl)-1H- | | | |
| | pyrazolo[4,3-d]pyrimidin-7- | | | |
| | yl)amino)hexan-1-ol | | | |
| 109 | 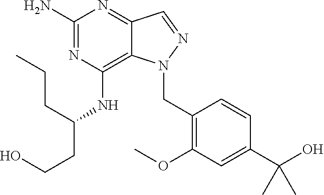 [SMILES: CCC[C@@H](CCO)Nc1nc(N)nc2cnn(Cc3ccc(C(C)(C)O)cc3OC)c12] | 4.6 | 3.5 | [M + H]⁺ = 429.0 LC RT = 1.36 (LC/MS Method A) δ 7.61 (s, 1H), 7.16 (d, J = 1.7 Hz, 1H), 6.93-6.87 (m, 1H), 6.46 (d, J = 8.0 Hz, 1H), 6.16 (s, 1H), 6.08 (s, 0H), 5.68 (d, J = 16.7 Hz, 1H), 5.57 (d, J = 16.8 Hz, 1H), 4.37 (s, 1H), 3.51 (s, 1H), 3.33 (s, 1H), 2.55 (d, J = 1.2 Hz, 5H), 1.65 (t, J = 6.8 Hz, 1H), 1.56-1.49 (m, 0H), 1.39 (s, 6H), 1.07 (dt, J = 22.6, 8.5 Hz, 2H), 0.78 (t, J = 7.3 Hz, 3H). |
| | (3S)-3-[(5-amino-1-{[4-(2-hydroxy- | | | |
| | propan-2-yl)-2-methoxyphenyl]- | | | |
| | methyl}-1H-pyrazolo[4,3-d] | | | |
| | pyrimidin-7-yl)amino]hexan-1-ol | | | |
| 110 | 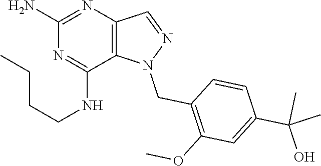 [SMILES: CCCCNc1nc(N)nc2cnn(Cc3ccc(C(C)(C)O)cc3OC)c12] | 71.13 | | [M + H]⁺ = 385.12 LC RT = 1.52 min (LC/MS Method A) |
| | 2-(4-{[5-amino-7-(butylamino)-1H- | | | |
| | pyrazolo[4,3-d]pyrimidin-1-yl] | | | |
| | methyl}-3-methoxyphenyl)propan-2-ol | | | |
| 111 | 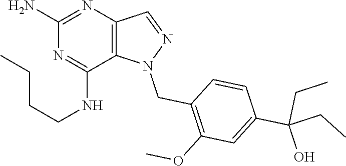 [SMILES: CCCCNc1nc(N)nc2cnn(Cc3ccc(C(O)(CC)CC)cc3OC)c12] | 45.85 | | [M − H]⁻ = 411.09 LC RT = 1.69 min (LC/MS Method A) |
| | 3-(4-{[5-amino-7-(butylamino)-1H- | | | |
| | pyrazolo[4,3-d]pyrimidin-1-yl] | | | |
| | methyl}-3-methoxyphenyl)pentan-3-ol | | | |
| 112 | 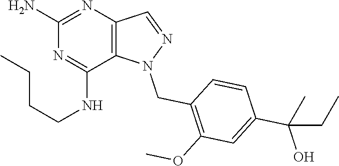 [SMILES: CCCCNc1nc(N)nc2cnn(Cc3ccc(C(C)(O)CC)cc3OC)c12] | 95.95 | | [M + H]⁺ = 399.37 LC RT = 1.52 min (LC/MS Method A) δ 7.57 (s, 1H), 7.11 (s, 1H), 6.94-6.78 (m, J = 7.9 Hz, 1H), 6.55-6.43 (m, 2H), 5.78 (br s, 2H), 5.61 (s, 2H), 3.84 (s, 3H), 1.66 (dt, J = 13.2, 6.7 Hz, 2H), 1.48 (quin, J = 7.2 Hz, 2H), 1.37 (s, 3H), 1.21 (dq, J = 14.7, 7.2 Hz, 2H), 0.85 (t, J = 7.3 Hz, 3H), 0.67 (br t, J = 7.3 Hz, 3H) |
| | 2-(4-{[5-amino-7-(butylamino)-1H- | | | |
| | pyrazolo[4,3-d]pyrimidin-1-yl] | | | |
| | methyl}-3-methoxyphenyl)butan-2-ol | | | |
| 113 | 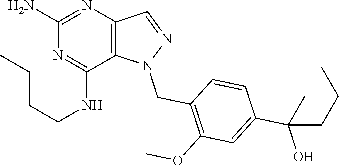 [SMILES: CCCCNc1nc(N)nc2cnn(Cc3ccc(C(C)(O)CCC)cc3OC)c12] | 255.54 | | [M + H]⁺ = 413.16 LC RT = 1.66 min (LC/MS Method A) δ 7.56 (s, 1H), 7.11 (s, 1H), 6.83 (br d, J = 7.3 Hz, 1H), 6.45 (br d, J = 8.2 Hz, 2H), 5.75 (br s, 1H), 5.60 (s, 2H), 3.84 (s, 3H), 1.61 (br dd, J = 10.8, 5.3 Hz, 2H), 1.47 (quin, J = 7.2 Hz, 2H), 1.37 (s, 3H), 1.31- 1.16 (m, 3H), 0.98 (br s, 1H), 0.85 (t, J = 7.3 Hz, 3H), 0.77 (t, J = 7.3 Hz, 3H) |
| | 2-(4-{[5-amino-7-(butylamino)-1H- | | | |
| | pyrazolo[4,3-d]pyrimidin-1-yl] | | | |
| | methyl}-3-methoxyphenyl)pentan-2-ol | | | |
| 114 | 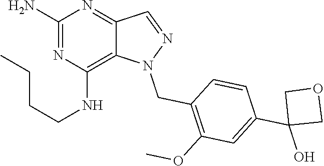 [SMILES: CCCCNc1nc(N)nc2cnn(Cc3ccc(C4(O)COC4)cc3OC)c12] | 410.23 | | [M + H]⁺ = 399.22 LC RT = 1.39 min (LC/MS Method A) δ 7.56 (s, 1H), 7.21 (s, 1H), 7.08 (d, J = 7.9 Hz, 1H), 6.57 (d, J = 7.9 Hz, 1H), 6.46 (br t, J = 5.5 Hz, 1H), 5.62 (br d, J = 8.9 Hz, 4H), 4.74 (d, J = 6.4 Hz, 2H), 4.66 (d, J = 6.4 Hz, 2H), 3.87 (s, 2H), 3.40 (br d, J = 6.1 Hz, 1H), 1.61-1.41 (m, 2H), 1.30-1.13 (m, 2H), 0.84 (t, J = 7.3 Hz, 3H) |
| | 3-(4-{[5-amino-7-(butylamino)-1H- | | | |
| | pyrazolo[4,3-d]pyrimidin-1-yl] | | | |
| | methyl}-3-methoxyphenyl)oxetan-3-ol | | | |
| 115 | 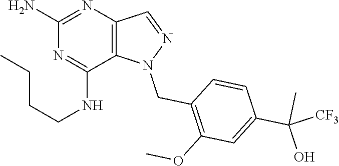 [SMILES: CCCCNc1nc(N)nc2cnn(Cc3ccc(C(C)(O)C(F)(F)F)cc3OC)c12] | 146.05 | 340.47 | [M + H]⁺ = 439.12 LC RT = 1.58 min (LC/MS Method A) δ 7.57 (s, 1H), 7.23 (s, 1H), 7.02 (br d, J = 7.9 Hz, 1H), 6.47 (d, J = 7.9 Hz, 2H), 5.64 (br d, J = 4.6 Hz, 4H), 3.86 (s, 3H), 3.20-3.14 (m, J = 14.0 Hz, 1H), 1.58 (br s, 1H), 1.47 (quin, J = 7.2 Hz, 2H), 1.38-1.26 (m, 1H), 1.18 (sxt, J = 7.4 Hz, 2H), 0.94 (t, J = 7.3 Hz, 2H), 0.83 (t, J = 7.5 Hz, 3H) |
| | 2-(4-{[5-amino-7-(butylamino)-1H- | | | |
| | pyrazolo[4,3-d]pyrimidin-1- | | | |
| | yl]methyl}-3-methoxyphenyl)-1,1,1- | | | |
| | trifluoropropan-2-ol | | | |
| 116 | 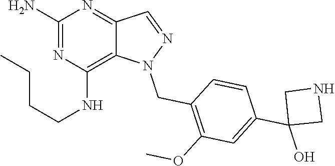 [SMILES: CCCCNc1nc(N)nc2cnn(Cc3ccc(C4(O)CNC4)cc3OC)c12] | 786.5 | | [M + H]⁺ = 398.05 LC RT = 1.05 min (LC/MS Method A) δ 7.55 (s, 1H), 7.20 (br s, 1H), 7.04 (br d, J = 7.5 Hz, 1H), 6.56 (br d, J = 7.7 Hz, 1H), 5.61 (s, 4H), 3.93-3.66 (m, 8H), 3.42- 3.36 (m, 2H), 1.94-1.75 (m, 2H), 1.51- 1.43 (m, 2H), 1.24-1.13 (m, 2H), 0.83 (t, J = 7.4 Hz, 3H) |
| | 3-(4-{[5-amino-7-(butylamino)-1H- | | | |
| | pyrazolo[4,3-d]pyrimidin-1- | | | |
| | yl]methyl}-3-methoxyphenyl)- | | | |
| | azetidin-3-ol | | | |
| 117 | 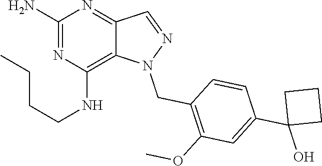 [SMILES: CCCCNc1nc(N)nc2cnn(Cc3ccc(C4(O)CCC4)cc3OC)c12] | 25.9 | 803.5 | [M + H]⁺ = 397.11 LC RT = 1.59 min (LC/MS Method A) δ 7.60 (s, 1H), 7.11 (s, 1H), 6.97 (d, J = 7.9 Hz, 1H), 6.58 (d, J = 7.9 Hz, 1H), 5.64 (s, 2H), 3.84 (s, 2H), 3.56-3.41 (m, 1H), 2.42- 2.31 (m, 2H), 2.28-2.21 (m, 2H), 1.94- 1.85 (m, 1H), 1.67-1.57 (m, 1H), 1.50 (quin, J = 7.2 Hz, 2H), 1.21 (sxt, J = 7.4 Hz, 2H), 0.85 (t, J = 7.3 Hz, 3H) |
| | 1-(4-{[5-amino-7-(butylamino)-1H- | | | |
| | pyrazolo[4,3-d]pyrimidin-1-yl] | | | |
| | methyl}-3-methoxyphenyl)cyclobutan- | | | |
| | 1-ol | | | |
| 118 | 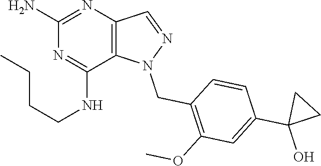 [SMILES: CCCCNc1nc(N)nc2cnn(Cc3ccc(C4(O)CC4)cc3OC)c12] | 257.4 | 275.7 | [M + H]⁺ = 383.17 LC RT = 1.7 min (LC/MS Method A) δ 7.63 (s, 1H), 7.50 (s, 1H), 7.48 (d, J = 7.8 Hz, 1H), 6.85 (br s, 1H), 6.53 (d, J = 7.6 Hz, 1H), 6.02 (br s, 1H), 5.73 (s, 2H), 3.91 (s, 3H), 3.00 (q, J = 7.2 Hz, 2H), 1.47 (quin, J = 7.3 Hz, 2H), 1.19-1.10 (m, 2H), 1.07 (t, J = 7.2 Hz, 3H), 0.81 (t, J = 7.5 Hz, 3H) |
| | 1-(4-{[5-amino-7-(butylamino)-1H- | | | |
| | pyrazolo[4,3-d]pyrimidin-1-yl] | | | |
| | methyl}-3-methoxyphenyl) | | | |
| | cyclopropan-1-ol | | | |
| 119 | 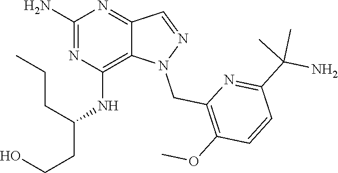 [SMILES: CCC[C@@H](CCO)Nc1nc(N)nc2cnn(Cc3nc(C(C)(C)N)ccc3OC)c12] | 4954 | | LC/MS [M + H]⁺ 429.2 LC RT: 1.04 min. (Method A). δ 7.52 (s, 1H), 7.47-7.38 (m, 2H), 6.16 (br d, J = 8.9 Hz, 1H), 5.82 (br d, J = 16.8 Hz, 1H), 5.70 (br d, J = 16.8 Hz, 1H), 5.53 (s, 2H), 4.29 (br s, 1H), 3.84 (s, 3H), 3.49 (br s, 1H), 3.32 (br s, 1H), 1.90 (br s, 2H), 1.70-1.53 (m, 2H), 1.53-1.35 (m, 3H), 1.16 (s, 3H), 1.13-1.10 (m, 3H), 1.10- 1.01 (m, 2H), 0.76 (t, J = 7.3 Hz, 3H) |
| | (3S)-3-[(5-amino-1-{[6-(2-amino- | | | |
| | propan-2-yl)-3-methoxypyridin-2- | | | |
| | yl]methyl}-1H-pyrazolo[4,3- | | | |
| | d]pyrimidin-7-yl)amino]hexan-1-ol | | | |
| 120 | 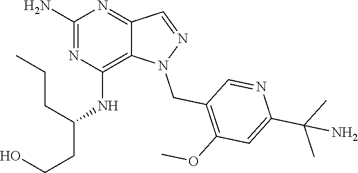 [SMILES: CCC[C@@H](CCO)Nc1nc(N)nc2cnn(Cc3cnc(C(C)(C)N)cc3OC)c12] | 48.3 | | LC/MS m/z = 429.2 [M + H]⁺ RT (min) = 0.857 (LC/MS method B). ¹H NMR (400 MHz, DMSO-d₆) δ = 7.59 (s, 1H), 7.46 (s, 1H), 7.27 (s, 1H), 6.24-6.18 (m, 1H), 5.76-5.58 (m, 4H), 4.40-4.29 (m, 1H), 3.91 (s, 3H), 3.39 (br s, 2H), 1.74- 1.59 (m, 2H), 1.54-1.41 (m, 8H), 1.20- 1.04 (m, 2H), 0.81 (t, J = 7.2 Hz, 3H). |
| | (3S)-3-[(5-amino-1-{[6-(2-amino- | | | |
| | propan-2-yl)-4-methoxypyridin-3- | | | |
| | yl]methyl}-1H-pyrazolo[4,3- | | | |
| | d]pyrimidin-7-yl)amino]hexan-1-ol | | | |

### Pharmaceutical Compositions and Administration

In another aspect, there is provided a pharmaceutical composition comprising a compound of as disclosed herein, or of a conjugate thereof, formulated together with a pharmaceutically acceptable carrier or excipient. It may optionally contain one or more additional pharmaceutically active ingredients, such as a biologic or a small molecule drug. The pharmaceutical compositions can be administered in a combination therapy with another therapeutic agent, especially an anti-cancer agent.

The pharmaceutical composition may comprise one or more excipients. Excipients that may be used include carriers, surface active agents, thickening or emulsifying agents, solid binders, dispersion or suspension aids, solubilizers, colorants, flavoring agents, coatings, disintegrating agents, lubricants, sweeteners, preservatives, isotonic agents, and combinations thereof. The selection and use of suitable excipients is taught in Gennaro, ed., Remington: The Science and Practice of Pharmacy, 20th Ed. (Lippincott Williams & Wilkins 2003).

Preferably, a pharmaceutical composition is suitable for intravenous, intramuscular, subcutaneous, parenteral, spinal or epidermal administration (e.g., by injection or infusion). Depending on the route of administration, the active compound may be coated in a material to protect it from the action of acids and other natural conditions that may inactivate it. The phrase “parenteral administration” means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, the pharmaceutical composition can be administered via a non-parenteral route, such as a topical, epidermal or mucosal route of administration, for example, intranasally, orally, vaginally, rectally, sublingually or topically.

Pharmaceutical compositions can be in the form of sterile aqueous solutions or dispersions. They can also be formulated in a microemulsion, liposome, or other ordered structure suitable to achieve high drug concentration. The compositions can also be provided in the form of lyophilates, for reconstitution in water prior to administration.

The amount of active ingredient which can be combined with a carrier material to produce a single dosage form will vary depending upon the subject being treated and the particular mode of administration and will generally be that amount of the composition which produces a therapeutic effect. Generally, out of one hundred percent, this amount will range from about 0.01 percent to about ninety-nine percent of active ingredient, preferably from about 0.1 percent to about 70 percent, most preferably from about 1 percent to about 30 percent of active ingredient in combination with a pharmaceutically acceptable carrier.

Dosage regimens are adjusted to provide a therapeutic response. For example, a single bolus may be administered, several divided doses may be administered overtime, or the dose may be proportionally reduced or increased as indicated by the exigencies of the situation. It is especially advantageous to formulate parenteral compositions in dosage unit form for ease of administration and uniformity of dosage. “Dosage unit form” refers to physically discrete units suited as unitary dosages for the subjects to be treated; each unit containing a predetermined quantity of active compound calculated to produce the desired therapeutic response, in association with the required pharmaceutical carrier.

The dosage ranges from about 0.0001 to 100 mg/kg, and more usually 0.01 to 5 mg/kg, of the host body weight. For example dosages can be 0.3 mg/kg body weight, 1 mg/kg body weight, 3 mg/kg body weight, 5 mg/kg body weight or 10 mg/kg body weight or within the range of 1-10 mg/kg, or alternatively 0.1 to 5 mg/kg. Exemplary treatment regimens are administration once per week, once every two weeks, once every three weeks, once every four weeks, once a month, once every 3 months, or once every three to 6 months. Preferred dosage regimens include 1 mg/kg body weight or 3 mg/kg body weight via intravenous administration, using one of the following dosing schedules: (i) every four weeks for six dosages, then every three months; (ii) every three weeks; (iii) 3 mg/kg body weight once followed by 1 mg/kg body weight every three weeks. In some methods, dosage is adjusted to achieve a plasma antibody concentration of about 1-1000 g/mL and in some methods about 25-300 μg/mL.

A “therapeutically effective amount” of a compound of the invention preferably results in a decrease in severity of disease symptoms, an increase in frequency and duration of disease symptom-free periods, or a prevention of impairment or disability due to the disease affliction. For example, for the treatment of tumor-bearing subjects, a “therapeutically effective amount” preferably inhibits tumor growth by at least about 20%, more preferably by at least about 40%, even more preferably by at least about 60%, and still more preferably by at least about 80% relative to untreated subjects. A therapeutically effective amount of a therapeutic compound can decrease tumor size, or otherwise ameliorate symptoms in a subject, which is typically a human but can be another mammal. Where two or more therapeutic agents are administered in a combination treatment, “therapeutically effective amount” refers to the efficacy of the combination as a whole, and not each agent individually.

The pharmaceutical composition can be a controlled or sustained release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. See, e.g., Sustained and Controlled Release Drug Delivery Systems, J. R. Robinson, ed., Marcel Dekker, Inc., New York, 1978.

Therapeutic compositions can be administered via medical devices such as (1) needleless hypodermic injection devices; (2) micro-infusion pumps; (3) transdermal devices; (4) infusion devices; and (5) osmotic devices.

In certain embodiments, the pharmaceutical composition can be formulated to ensure proper distribution in vivo. For example, to ensure that the therapeutic compounds of the invention cross the blood-brain barrier, they can be formulated in liposomes, which may additionally comprise targeting moieties to enhance selective transport to specific cells or organs.

### INDUSTRIAL APPLICABILITY AND USES

TLR7 agonist compounds disclosed herein can be used for the treatment of a disease or condition that can be ameliorated by activation of TLR7.

In one embodiment, the TLR7 agonist is used in combination with an anti-cancer immunotherapy agent—also known as an immuno-oncology agent. An anti-cancer immunotherapy agent works by stimulating a body's immune system to attack and destroy cancer cells, especially through the activation of T cells. The immune system has numerous checkpoint (regulatory) molecules, to help maintain a balance between its attacking legitimate target cells and preventing it from attacking healthy, normal cells. Some are stimulators (up-regulators), meaning that their engagement promotes T cell activation and enhances the immune response. Others are inhibitors (down-regulators or brakes), meaning that their engagement inhibits T cell activation and abates the immune response. Binding of an agonistic immunotherapy agent to a stimulatory checkpoint molecule can lead to the latter's activation and an enhanced immune response against cancer cells. Reciprocally, binding of an antagonistic immunotherapy agent to an inhibitory checkpoint molecule can prevent down-regulation of the immune system by the latter and help maintain a vigorous response against cancer cells. Examples of stimulatory checkpoint molecules are B7-1, B7-2, CD28, 4-1BB (CD137), 4-1BBL, ICOS, CD40, ICOS-L, OX40, OX40L, GITR, GITRL, CD70, CD27, CD40, DR3 and CD28H. Examples of inhibitory checkpoint molecules are CTLA-4, PD-1, PD-L1, PD-L2, LAG-3, TIM-3, Galectin 9, CEACAM-1, BTLA, CD69, Galectin-1, CD113, GPR56, VISTA, 2B4, CD48, GARP, PD1H, LAIR1, TIM-1, CD96 and TIM-4.

Whichever the mode of action of an anti-cancer immunotherapy agent, its effectiveness can be increased by a general up-regulation of the immune system, such as by the activation of TLR7. Thus, in one embodiment, this specification provides a method of treating a cancer, comprising administering to a patient suffering from such cancer a therapeutically effective combination of an anti-cancer immunotherapy agent and a TLR7 agonist as disclosed herein. The timing of administration can be simultaneous, sequential, or alternating. The mode of administration can systemic or local. The TLR7 agonist can be delivered in a targeted manner, via a conjugate.

Cancers that could be treated by a combination treatment as described above include acute myeloid leukemia, adrenocortical carcinoma, Kaposi sarcoma, lymphoma, anal cancer, appendix cancer, teratoid/rhabdoid tumor, basal cell carcinoma, bile duct cancer, bladder cancer, bone cancer, brain cancer, breast cancer, bronchial tumor, carcinoid tumor, cardiac tumor, cervical cancer, chordoma, chronic lymphocytic leukemia, chronic myeloproliferative neoplasm, colon cancer, colorectal cancer, craniopharyngioma, bile duct cancer, endometrial cancer, ependymoma, esophageal cancer, esthesioneuroblastoma, Ewing sarcoma, eye cancer, fallopian tube cancer, gallbladder cancer, gastrointestinal carcinoid tumor, gastrointestinal stromal tumor, germ cell tumor, hairy cell leukemia, head and neck cancer, heart cancer, liver cancer, hypopharngeal cancer, pancreatic cancer, kidney cancer, laryngeal cancer, chronic myelogenous leukemia, lip and oral cavity cancer, lung cancer, melanoma, Merkel cell carcinoma, mesothelioma, mouth cancer, oral cancer, osteosarcoma, ovarian cancer, penile cancer, pharyngeal cancer, prostate cancer, rectal cancer, salivary gland cancer, skin cancer, small intestine cancer, soft tissue sarcoma, testicular cancer, throat cancer, thyroid cancer, urethral cancer, uterine cancer, vaginal cancer, and vulvar cancer.

Anti-cancer immunotherapy agents that can be used in combination therapies as disclosed herein include: AMG 557, AMP-224, atezolizumab, avelumab, BMS 936559, cemiplimab, CP-870893, dacetuzumab, durvalumab, enoblituzumab, galiximab, IMP321, ipilimumab, lucatumumab, MEDI-570, MEDI-6383, MEDI-6469, muromonab-CD3, nivolumab, pembrolizumab, pidilizumab, spartalizumab, tremelimumab, urelumab, utomilumab, varlilumab, vonlerolizumab. Table B below lists their alternative name(s) (brand name, former name, research code, or synonym) and the respective target checkpoint molecule.

TABLE B

| Immunotherapy Agent | Alternative Name(s) | Target |
|---|---|---|
| AMG 557 | | B7RP-1 (ICOSL) |
| AMP-224 | | PD-1 |
| Atezolizumab | MPDL3280A, RO5541267, | PD-L1 |
| | TECENTRIQ ® | |
| Avelumab | BAVENCIO ® | PD-L1 |
| BMS 936559 | | PD-L1 |
| Cemiplimab | LIBTAYO ® | PD-1 |
| CP-870893 | | CD40 |
| Dacetuzumab | | CD40 |
| Durvalumab | IMFINZI ® | PD-L1 |
| Enoblituzumab | MGA271 | B7-H3 |
| Galiximab | | B7-1 (CD80) |
| IMP321 | | LAG-3 |
| Ipilimumab | YERVOY ® | CTLA-4 |
| Lucatumumab | | CD40 |
| MEDI-570 | | ICOS (CD278) |
| MEDI-6383 | | OX40 |
| MEDI-6469 | | OX40 |
| Muromonab-CD3 | | CD3 |
| Nivolumab | OPDIVO ® | PD-1 |
| Pembrolizumab | KEYTRUDA ® | PD-1 |
| Pidilizumab | MDV9300 | PD-1 |
| Spartalizumab | PDR001 | PD-1 |
| Tremelimumab | Ticilimumab, CP-675, | CTLA-4 |
| | CP-675, 206 | |
| Urelumab | BMS-663513 | CD137 |
| Utomilumab | PF-05082566 | CD137 |
| Varlilumab | CDX 1127 | CD27 |
| Vonlerolizumab | RG7888, MOXR0916, | OX40 |
| | pogalizumab | |

In one embodiment of a combination treatment with a TLR7 agonist, the anti-cancer immunotherapy agent is an antagonistic anti-CTLA-4, anti-PD-1, or anti-PD-L1 antibody. The cancer can be lung cancer (including non-small cell lung cancer), pancreatic cancer, kidney cancer, head and neck cancer, lymphoma (including Hodgkin's lymphoma), skin cancer (including melanoma and Merkel skin cancer), urothelial cancer (including bladder cancer), gastric cancer, hepatocellular cancer, or colorectal cancer.

In another embodiment of a combination treatment with a TLR7 agonist, the anti-cancer immunotherapy agent is an antagonistic anti-CTLA-4 antibody, preferably ipilimumab.

In another embodiment of a combination treatment with a TLR7 agonist, the anti-cancer immunotherapy agent is an antagonistic anti-PD-1 antibody, preferably nivolumab or pembrolizumab.

The TLR7 agonists disclosed herein also are useful as vaccine adjuvants.

The practice of this invention can be further understood by reference to the following examples, which are provided by way of illustration and not of limitation.

### Analytical Procedures

### NMR

The following conditions were used for obtaining proton nuclear magnetic resonance (NMR) spectra: NMR spectra were taken in either 400 Mz or 500 Mhz Bruker instrument using either DMSO-d6 or CDCl₃ as solvent and internal standard. The crude NMR data was analyzed by using either ACD Spectrus version 2015-01 by ADC Labs or MestReNova software.

Chemical shifts are reported in parts per million (ppm) downfield from internal tetramethylsilane (TMS) or from the position of TMS inferred by the deuterated NMR solvent. Apparent multiplicities are reported as: singlet-s, doublet-d, triplet-t, quartet-q, or multiplet-m. Peaks that exhibit broadening are further denoted as br. Integrations are approximate. It should be noted that integration intensities, peak shapes, chemical shifts and coupling constants can be dependent on solvent, concentration, temperature, pH, and other factors. Further, peaks that overlap with or exchange with water or solvent peaks in the NMR spectrum may not provide reliable integration intensities. In some cases, NMR spectra may be obtained using water peak suppression, which may result in overlapping peaks not being visible or having altered shape and/or integration.

### Liquid Chromatography

The following preparative and analytical (LC/MS) liquid chromatography methods were used:

Analytical LC/MS Procedure A: Column: Waters XBridge C18, 2.1 mm×50 mm, 1.7 μm particles; Mobile Phase A: 5:95 acetonitrile:water with 10 mM NH₄OAc; Mobile Phase B: 95:5 acetonitrile:water with 10 mM NH₄OAc; Temperature: 50° C.; Gradient: 0% B to 100% B over 3 min, then a 0.50 min hold at 100% B; Flow: 1 ml/min; Detection: MS and UV (220 nm).

Analytical LC/MS Procedure B: Column: Xbridge BEH C18 XP (50×2.1 mm), 2.5 μm; mobile phase A: 5:95 CH₃CN: H₂O with 10 mM NH₄OAc; mobile phase B: 95:5 CH₃CN: H₂O with 10 mM NH₄OAc; temperature: 50° C.; gradient: 0-100% B over 3 min; flow rate: 1.1 ml/min).

### Synthesis—General Procedures

Generally, the procedures disclosed herein produce a mixture of regioisomers, alkylated at the 1H or 2H position of the pyrazolopyrimidine ring system (which are also referred to as N1 and N2 regioisomers, respectively, alluding to the nitrogen that is alkylated). For brevity, the N2 regioisomers are not shown for convenience, but it is to be understood that they are present in the initial product mixture and separated at a later time, for example by preparative HPLC.

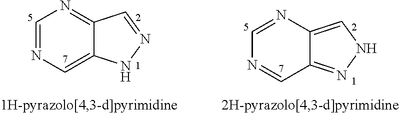 [SMILES: c1ncc2[nH]ncc2n1 ; c1ncc2n[nH]cc2n1]

The mixture of regioisomers can be separated at an early stage of the synthesis and the remaining synthetic steps carried out with the 1H regioisomer or, alternatively, the synthesis can be progressed carrying the mixture of regioisomers and separation effected at a later stage, as desired.

The compounds of the present disclosure can be prepared by a number of methods well known to one skilled in the art of synthetic organic chemistry. These methods include those described below, or variations thereof. Preferred methods include, but are not limited to, those described below in Schemes 1-4 below.

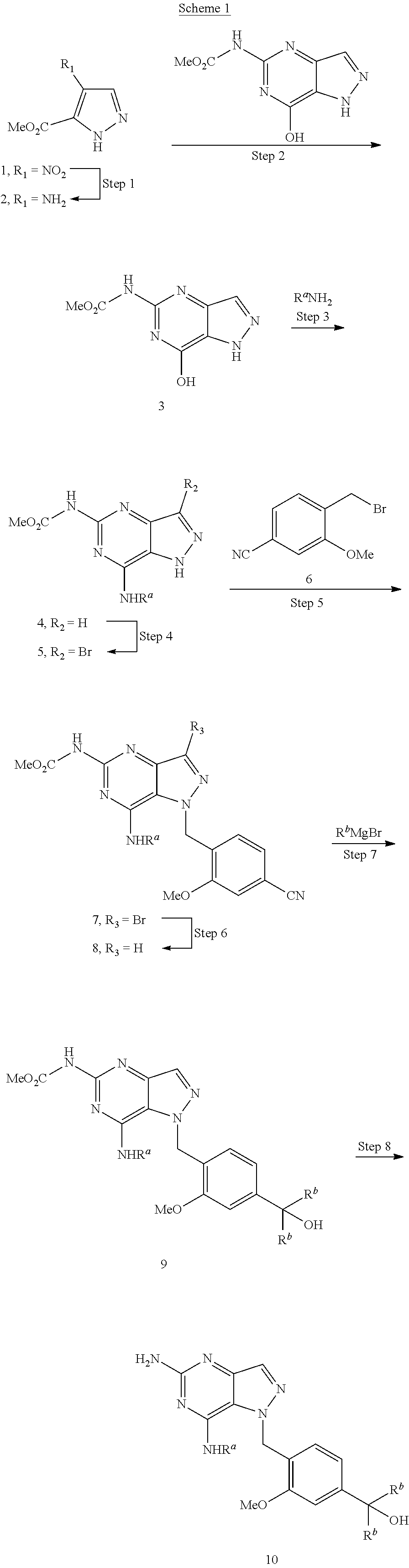

Compound 10 can be prepared by the synthetic sequence outlined in Scheme 1 above. Reduction of nitropyrazole 1 to give the corresponding amine 2, followed by cyclisation with 1,3-bis(methoxycarbonyl)-2-methyl-2-thiopseudourea gives the hydroxypyrazolopyrimidine 3. The amine is introduced using BOP/DBU coupling conditions to give compound 4. Subsequent bromination using NBS gives the bromopyrazolopyrimidine 5. Alkylation using benzyl halide 6 gives a mixture of N1 and N2 products, which are separated, giving N1 intermediate 7, which is debrominated using catalytic hydrogenation to give cyano intermediate 8, which is then converted to the gem-dialkylamine 9 using Grignard reagents and titanium (IV) isopropoxide. Finally, removal of the methyl carbamate using sodium hydroxide gives the target compound 10.

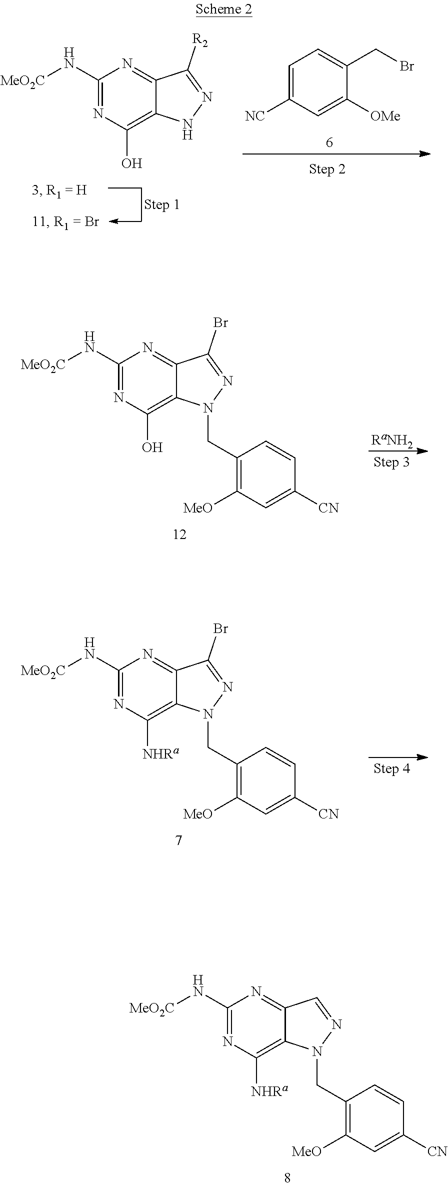

Alternatively, cyano intermediate 8 can be accessed using the route described in Scheme 2 above. Intermediate 3 is brominated using NBS to give compound 11, which is alkylated using the benzyl halide 6 to give the hydroxy intermediate 12. Incorporation of an amine R^{a}NH₂ using BOP/DBU followed by debromination gives the desired intermediate 8.

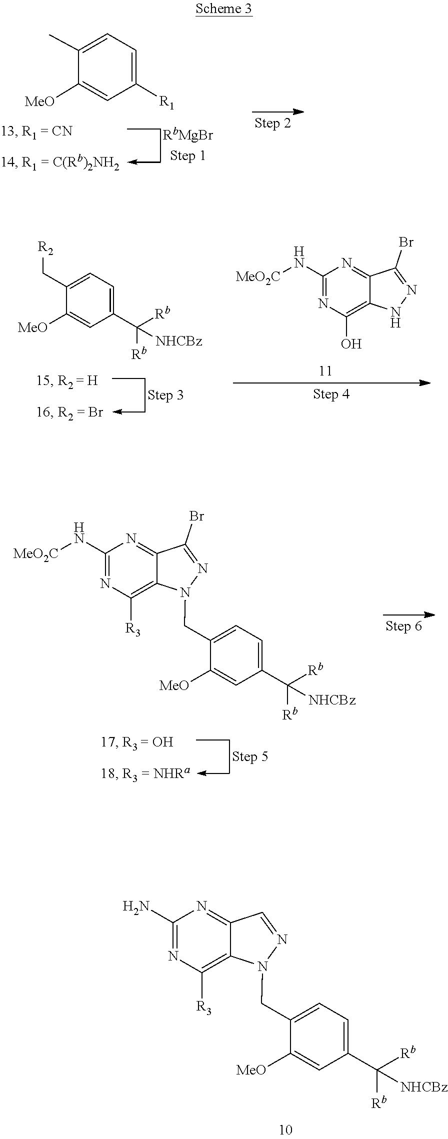

Another alternative route to compound 10 is described in Scheme 3 above. Benzonitrile 13 is converted to the dialkylamine 14 using a Grignard reagent and titanium (IV) isopropoxide. Amine 14 is then protected using benzyl chloroformate to give intermediate 15, which is converted to benzyl bromide 16 using NBS/AIBN. Benzyl bromide 16 is attached to the pyrazolopyrimidine intermediate 11 using Cs₂CO₃ in DMF. Resulting product 17 is aminated with R^{a}NH₂ using BOP/DBU. This gives intermediate 18, which is converted to the final compound 10 using catalytic hydrogenation to remove the bromo group and the CBz protecting group, followed by sodium hydroxide to remove the methyl carbamate.

The above Schemes are depicted in the regioisomerism of formula (I). Those skilled in the art will understand that the same Schemes can be adapted to make compounds according to formula (II), mutatis mutandis.

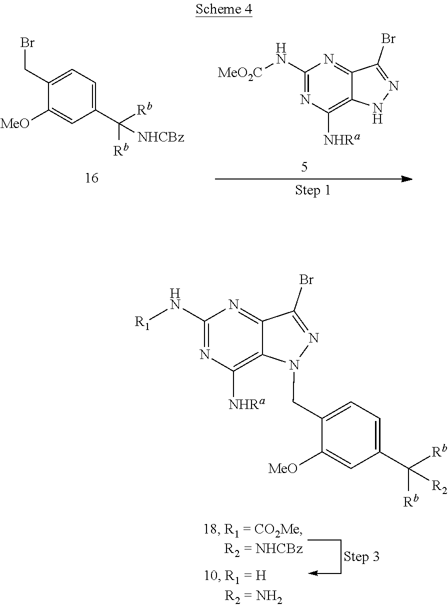

Scheme 4 above shows an alternative approach, in which intermediate 16 is attached to amine-containing pyrazolopyrimidine 5, giving intermediate 18. Deprotection of intermediate 18 as before using catalytic hydrogenation and sodium hydroxide gives target compound 10.

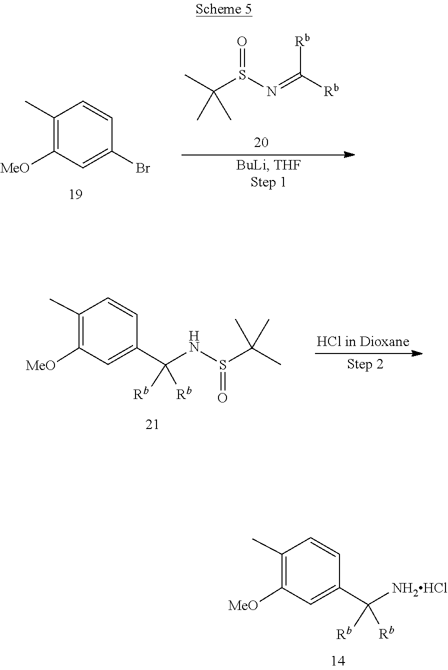

A further alternative to the preparation of intermediate amine 14 is illustrated in scheme 5. Benzyl halide 19 can be lithiated and quenched with sulfinamide 20, to afford the protected amine 21. Deprotection using HCl in dioxane gives the desired amine 14 as a hydrochloride salt.

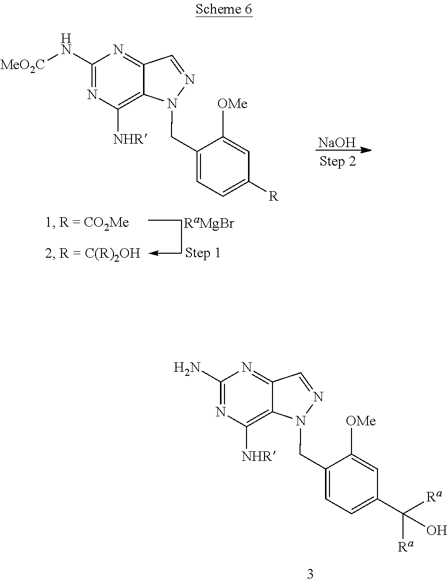

Symmetric tertiary alcohols 3 (both R^{a} groups the same) can be made per the above Scheme 6. Addition of the Grignard reagent R^{a}MgBr to compound 1 (US 2020/0038403), followed by removal of the methyl carbamate protecting group gives target compound 3.

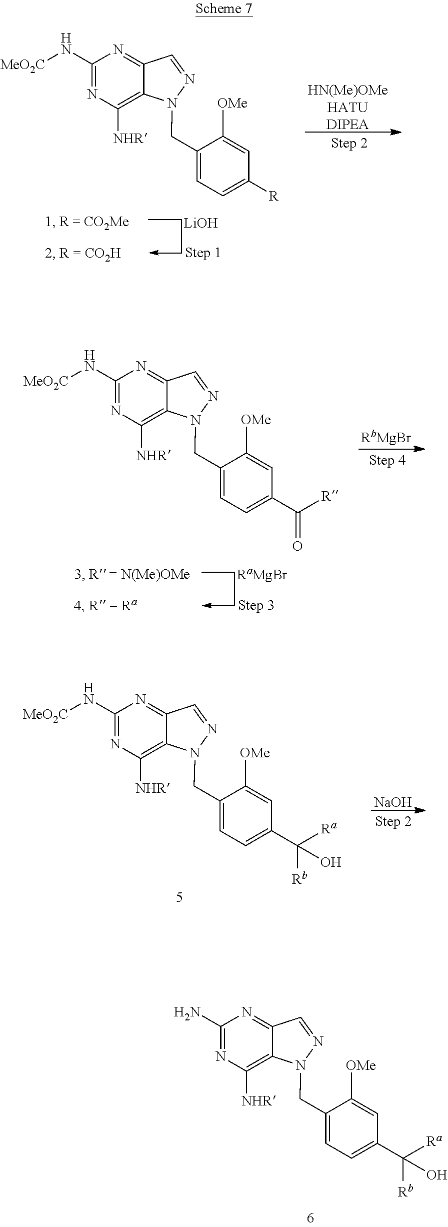

Unsymmetric tertiary alcohols can be made per the above Scheme 7. Ester 1 is hydrolyzed to acid 2, followed by conversion to Weinreb amide 3. Amide 3 is converted to ketone 4 with Grignard reagent R^{a}MgBr. Alkylation with a second Grignard reagent R^{b}MgBr and subsequent removal of the methyl carbamate protecting group yields asymmetric tertiary alcohol 6.

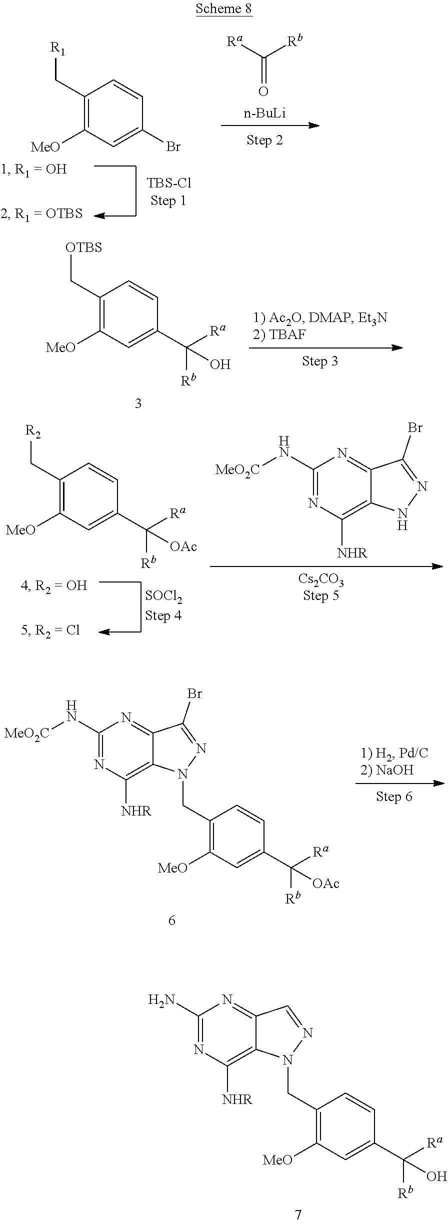

An alternative route to the tertiary alcohols is shown in the above Scheme 8. Benzyl alcohol 1 is protected (with a TBS group used as exemplar). Metallation of the bromine position in compound 2 with n-BuLi and quenching with a ketone R^{a}C(═O)R^{B} leads to alcohol 3. (R^{a} and R^{b} can be the same or different.) Protection of the tertiary alcohol (e.g., as acetate) and deprotection of the benzyl alcohol in Step 3 yields benzyl alcohol 4, which is converted to the corresponding benzyl chloride 6 with thionyl chloride. Coupling per step 5 yields compound 6. (The bromine group at C3 helps direct the coupling to the N1 position in preference over the N2 position.) Hydrogenation to remove the bromine and hydrolysis of the carbamate protecting group yields final product 7.

### Synthesis—Specific Examples

To further illustrate the foregoing, the following non-limiting, the following exemplary synthetic schemes are included. Variations of these examples within the scope of the claims are within the purview of one skilled in the art and are considered to fall within the scope of this disclosure. The reader will recognize that the skilled artisan, provided with the present disclosure and skilled in the relevant art, will be able to prepare and use the compounds disclosed herein without exhaustive examples.

Analytical data for compounds numbered 100 and higher is found in Table A.

### Example 1—Compound 101

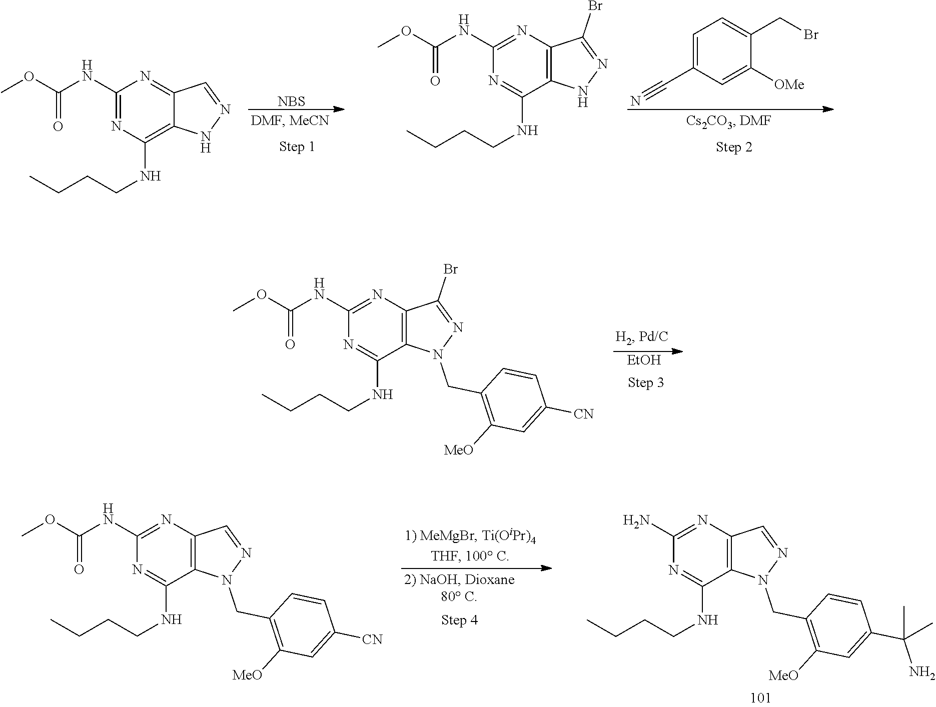

Step 1. To a stirred suspension of methyl (7-(butylamino)-1H-pyrazolo[4,3-d]pyrimidin-5-yl)carbamate (10 g, 37.8 mmol) in DMF/MeCN (1:1, 120 mL) was added NBS (7.41 g, 41.6 mmol). The reaction was stirred at RT for 1 h. Water (150 mL) was added and the reaction mixture was stirred for a further 10 min. The product was collected by filtration and washed with water (3×50 mL), giving methyl (3-bromo-7-(butylamino)-1H-pyrazolo[4,3-d]pyrimidin-5-yl)carbamate (6.1 g, 17.77 mmol, 47.0% yield) as a solid.

LC-MS (ES, m/z): [M+H]⁺ 343.0, 345.0.

¹H NMR (400 MHz, DMSO-d₆) δ 12.87 (br s, 1H), 9.80 (s, 1H), 7.57 (br s, 1H), 3.62 (s, 3H), 3.59-3.48 (m, 2H), 1.62 (quin, J=7.2 Hz, 2H), 1.40 (dq, J=14.9, 7.3 Hz, 2H), 0.94 (t, J=7.4 Hz, 3H)

Step 2. To a stirred suspension of methyl (3-bromo-7-(butylamino)-1H-pyrazolo[4,3-d]pyrimidin-5-yl)carbamate (1 g, 2.91 mmol) and Cs₂CO₃ (1.899 g, 5.83 mmol) in DMF (10 mL) at 0° C. was added a solution of 4-(bromomethyl)-3-methoxybenzonitrile (0.527 g, 2.331 mmol) in DMF (2 mL). The reaction mixture was allowed to warm slowly to RT, stirred overnight, poured into saturated NaHCO₃ solution (100 mL), and extracted with EtOAc (3×40 mL). The combined organic phases were washed with brine (3×40 mL), dried (MgSO₄), filtered, and concentrated. Flash chromatography (80 g SiO₂ column, 0 to 70% EtOAc in hexane) gave methyl (3-bromo-7-(butylamino)-1-(4-cyano-2-methoxybenzyl)-1H-pyrazolo[4,3-d]pyrimidin-5-yl)carbamate (320 mg, 0.655 mmol, 22.49% yield) as a solid.

LC-MS (ES, m/z): [M+H]⁺ 488.3, 490.3.

¹H NMR (400 MHz, DMSO-d6) δ 9.88 (s, 1H), 7.53 (s, 1H), 7.39-7.32 (m, 2H), 6.77 (d, J=7.7 Hz, 1H), 5.78 (s, 2H), 3.85 (s, 3H), 3.63 (s, 3H), 3.51 (q, J=6.6 Hz, 2H), 1.55 (quin, J=7.3 Hz, 2H), 1.21 (sxt, J=7.4 Hz, 2H), 0.86 (t, J=7.4 Hz, 3H).

Step 3. Methyl (3-bromo-7-(butylamino)-1-(4-cyano-2-methoxybenzyl)-1H-pyrazolo[4,3-d]pyrimidin-5-yl)carbamate (315 mg, 0.645 mmol) was suspended in EtOH (15 mL). 10% palladium on carbon (15 mg) was added. The reaction vessel evacuated and purged with hydrogen six times. The reaction mixture was stirred under a hydrogen atmosphere for 2 h, filtered, and evaporated to dryness, giving methyl (7-(butylamino)-1-(4-cyano-2-methoxybenzyl)-1H-pyrazolo[4,3-d]pyrimidin-5-yl)carbamate (264 mg, 0.645 mmol, 98% yield) as a solid.

LC-MS (ES, m/z): [M+H]⁺ 410.4.

¹H NMR (400 MHz, CHLOROFORM-d) δ 9.99 (br s, 1H), 8.73 (br s, 1H), 8.03 (s, 1H), 7.25-7.18 (m, 2H), 7.11 (s, 1H), 6.12 (s, 2H), 3.89 (s, 3H), 3.86 (s, 3H), 3.80 (q, J=6.8 Hz, 2H), 1.64 (quin, J=7.4 Hz, 2H), 1.34-1.21 (m, 2H), 0.89 (t, J=7.4 Hz, 3H)

Step 4. A microwave vial was charged with methyl (7-(butylamino)-1-(4-cyano-2-methoxybenzyl)-1H-pyrazolo[4,3-d]pyrimidin-5-yl)carbamate (60 mg, 0.147 mmol) and THF (2 mL). Methylmagnesium bromide (0.293 mL, 0.879 mmol) was added. After the effervescence ceased, the vial was capped and the reaction mixture heated to 100° C. for 10 min in a microwave oven. Titanium(IV) isopropoxide (42 mg, 0.147 mmol) and methylmagnesium bromide (0.293 mL, 0.879 mmol) were added, and the reaction mixture heated for a further 10 min at 100° C. After cooling, the reaction mixture was quenched with saturated NH₄Cl solution (20 mL) and extracted with EtOAc (3×5 mL). The combined organic phases were washed with brine (2×5 mL), dried (MgSO₄), filtered, and concentrated. The residue was dissolved in dioxane (2 mL) and NaOH (0.440 mL, 2.198 mmol) was added. The reaction mixture was heated for 3 hours at 80° C. After cooling, the reaction mixture was neutralized with 5N HCl and evaporated to dryness. The crude material was dissolved in DMF (2 mL), filtered and purified via preparative LC/MS with the following conditions: Column: XBridge C18, 200 mm×19 mm, 5-μm particles; Mobile Phase A: 5:95 acetonitrile: water with NH₄OAc; Mobile Phase B: 95:5 acetonitrile: water with NH₄OAc; Gradient: a 0-minute hold at 2% B, 2-42% B over 20 min, then a 0-minute hold at 100% B; Flow Rate: 20 mL/min; Column Temperature: 25 C. Fraction collection was triggered by MS signals. Fractions containing the desired product were combined and dried via centrifugal evaporation, giving Compound 101 (9.2 mg, 0.024 mmol, 16% yield). Example 2—Compound 102

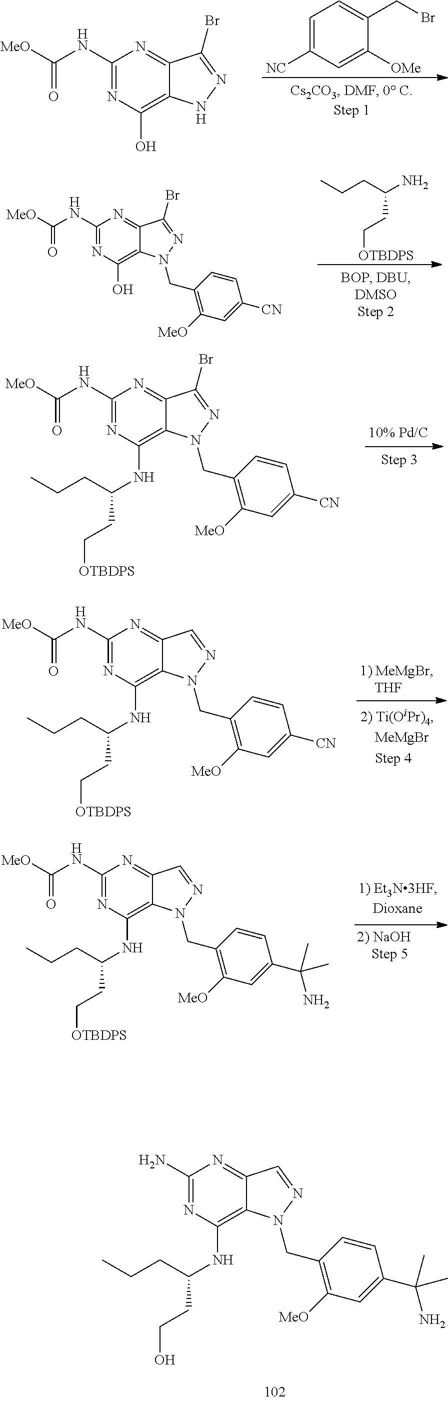

Step 1. To a stirred solution of methyl (3-bromo-7-hydroxy-1H-pyrazolo[4,3-d]pyrimidin-5-yl)carbamate (2 g, 6.94 mmol) in DMF (40 mL) was added Cs₂CO₃ (2.488 g, 7.64 mmol). After cooling in an ice bath, a solution of 4-(bromomethyl)-3-methoxybenzonitrile (1.570 g, 6.94 mmol) in DMF (10 mL) was added. The reaction was allowed to warm slowly to RT and stirred overnight. The reaction mixture was poured into saturated NaHCO₃ solution (200 mL) and water (200 mL), and EtOAc (200 mL) was added. With no separation of layers due to very poor solubility, the mixture was filtered. The precipitate was washed with water (2×50 mL) and MeCN (2×50 mL), giving methyl (3-bromo-1-(4-cyano-2-methoxybenzyl)-7-hydroxy-1H-pyrazolo[4,3-d]pyrimidin-5-yl)carbamate (1.1 g, 2.54 mmol, 36.6% yield) as a solid. LC-MS (ES, m/z): [M−H]=431.1, 433.1.

¹H NMR (400 MHz, DMSO-d₆) δ 11.53 (br s, 2H), 7.60-7.49 (m, 1H), 7.43-7.29 (m, 1H), 6.92 (br d, J=7.7 Hz, 1H), 5.73 (s, 2H), 3.88 (s, 3H), 3.75 (s, 3H).

Step 2. To a stirred solution of methyl (3-bromo-1-(4-cyano-2-methoxybenzyl)-7-hydroxy-1H-pyrazolo[4,3-d]pyrimidin-5-yl)carbamate (1 g, 2.308 mmol), (S)-1-((tert-butyldiphenylsilyl)oxy)hexan-3-amine (1.231 g, 3.46 mmol) and BOP (1.531 g, 3.46 mmol) in DMSO (20 mL) was added DBU (1.044 mL, 6.92 mmol). The reaction was stirred at 60° C. for 1 hour. After cooling, the reaction mixture was poured into saturated NaHCO₃ solution (100 mL) and extracted into EtOAc (3×70 ml). The combined organics were washed with brine (4×40 ml), dried (MgSO₄), filtered and concentrated. The crude material was purified using flash chromatography (80 g SiO₂ column, 0 to 50% EtOAc in hexanes), giving methyl (S)-(3-bromo-7-((1-((tert-butyldiphenylsilyl)oxy)hexan-3-yl)amino)-1-(4-cyano-2-methoxybenzyl)-1H-pyrazolo[4,3-d]pyrimidin-5-yl)carbamate (1.091 g, 1.415 mmol, 61.3% yield) as an oil. LC-MS (ES, m/z): [M+H]⁺=770.3, 772.2.

¹H NMR (400 MHz, DMSO-d₆) δ 9.83 (s, 1H), 7.58-7.53 (m, 2H), 7.50-7.45 (m, 3H), 7.43-7.33 (m, 4H), 7.27-7.22 (m, 2H), 7.11 (dd, J=7.9, 1.3 Hz, 1H), 6.62 (d, J=8.1 Hz, 1H), 6.53 (d, J=7.7 Hz, 1H), 5.86-5.71 (m, 2H), 3.81 (s, 3H), 3.62-3.52 (m, 5H), 1.87-1.77 (m, 2H), 1.57-1.43 (m, 2H), 1.15 (br dd, J=17.6, 10.8 Hz, 2H), 0.92 (s, 9H), 0.79 (t, J=7.4 Hz, 3H).

Step 3. To a solution of methyl (S)-(3-bromo-7-((1-((tert-butyldiphenylsilyl)-oxy)hexan-3-yl)amino)-1-(4-cyano-2-methoxybenzyl)-1H-pyrazolo[4,3-d]pyrimidin-5-yl)carbamate (1.08 g, 1.401 mmol) in ethanol (70 mL) was added 10% Pd on carbon (100 mg). The reaction mixture was evacuated and purged six times with hydrogen, then the reaction was stirred under a hydrogen atmosphere for 1 hour. The reaction was filtered through CELITE™, washing with ethanol (50 mL) and the filtrate evaporated to dryness. The crude material was purified using flash chromatography (40 g SiO₂ column, 0 to 100% EtOAc in hexanes), giving 183 mg of product. The column was re-eluted (0 to 10% MeOH in DCM over 25 min), giving a further 435 mg of product. The two batched were combined to give methyl (S)-(7-((1-((tert-butyldiphenylsilyl)oxy)hexan-3-yl)amino)-1-(4-cyano-2-methoxybenzyl)-1H-pyrazolo[4,3-d]pyrimidin-5-yl)carbamate (618 mg, 0.893 mmol, 63.7% yield) as a solid. LC-MS (ES, m/z): [M+H]⁺=692.4

¹H NMR (400 MHz, DMSO-d₆) δ 9.55 (s, 1H), 7.95 (s, 1H), 7.55 (d, J=7.2 Hz, 2H), 7.49-7.34 (m, 7H), 7.23 (t, J=7.4 Hz, 2H), 7.01 (d, J=8.1 Hz, 1H), 6.36 (d, J=8.6 Hz, 1H), 6.29 (d, J=7.7 Hz, 1H), 5.81 (d, J=2.4 Hz, 2H), 4.57 (brd, J=7.5 Hz, 1H), 3.85 (s, 3H), 3.61-3.58 (m, 3H), 3.57-3.48 (m, 2H), 1.86-1.72 (m, 2H), 1.55-1.41 (m, 2H), 1.20-0.97 (m, 2H), 0.93 (s, 9H), 0.77 (t, J=7.4 Hz, 3H).

Step 4. A microwave vial was charged with methyl (S)-(7-((1-((tert-butyldiphenylsilyl)oxy)hexan-3-yl)amino)-1-(4-cyano-2-methoxybenzyl)-1H-pyrazolo[4,3-d]pyrimidin-5-yl)carbamate (150 mg, 0.217 mmol) and THF (7 mL). Methylmagnesium bromide (0.361 mL, 1.084 mmol) was added, and the vial was capped and the reaction heated in the microwave for 20 min at 80° C. Titanium(IV) isopropoxide (0.127 mL, 0.434 mmol) was added, followed by more methylmagnesium bromide (0.361 mL, 1.084 mmol). The reaction was heated for a further 20 min in the microwave at 80° C. The reaction mixture was quenched with NH₄Cl solution (10 mL) and extracted into EtOAc (3×5 mL). The combined organics were washed with brine (5 mL), dried (MgSO₄), filtered and concentrated, giving methyl (S)-(1-(4-(2-aminopropan-2-yl)-2-methoxybenzyl)-7-((1-((tert-butyldiphenylsilyl)oxy)hexan-3-yl)amino)-1H-pyrazolo[4,3-d]pyrimidin-5-yl)carbamate (150 mg, 0.145 mmol, 66.9% yield, 70% purity) as a gum.

LC-MS (ES, m/z): [M+H]⁺=724.4.

Step 5. Methyl (S)-(1-(4-(2-aminopropan-2-yl)-2-methoxybenzyl)-7-((1-((tert-butyldiphenylsilyl)oxy)hexan-3-yl)amino)-1H-pyrazolo[4,3-d]pyrimidin-5-yl)carbamate (150 mg, 0.145 mmol) was dissolved in dioxane (4 mL). Triethylamine trihydrofluoride (0.118 mL, 0.725 mmol) was added and the reaction stirred at 70° C. for 1 hour. NaOH (1.450 mL, 7.25 mmol) was then added, and the reaction stirred at 80° C. for a further hour. After cooling, the reaction mixture was neutralized with 5N HCl and evaporated to dryness. The residue was dissolved in DMF (2 ml), filtered and purified via preparative LC/MS with the following conditions: Column: XBridge C18, 200 mm×19 mm, 5-μm particles; Mobile Phase A: 5:95 acetonitrile: water with NH₄OAc; Mobile Phase B: 95:5 acetonitrile: water with NH₄OAc; Gradient: a 0-minute hold at 2% B, 2-42% B over 20 min, then a 0-minute hold at 100% B; Flow Rate: 20 mL/min; Column Temperature: 25 C. Fraction collection was triggered by MS and UV signals. Fractions containing the desired product were combined and dried via centrifugal evaporation, giving Compound 102 (22.2 mg, 0.050 mmol, 34.7% yield).

### Example 3—Compound 103

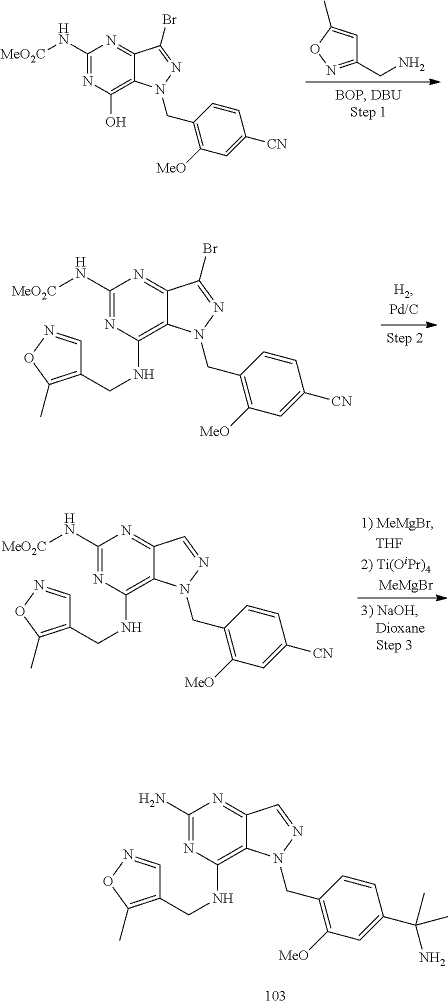

Step 1. To a stirred solution of methyl (3-bromo-1-(4-cyano-2-methoxybenzyl)-7-hydroxy-1H-pyrazolo[4,3-d]pyrimidin-5-yl)carbamate (742 mg, 1.713 mmol), (5-methylisoxazol-3-yl)methanamine (288 mg, 2.57 mmol) and BOP (1136 mg, 2.57 mmol) in DMSO (10 mL) was added DBU (0.775 mL, 5.14 mmol). The reaction was stirred at 60° C. for 1 hour. The reaction mixture was poured into saturated NaHCO₃ solution (100 mL) and extracted into EtOAc (3×70 mL). The combined organics were washed with brine (4×40 mL), dried (MgSO₄), filtered and concentrated. Flash chromatography (80 g SiO₂ column, 0 to 85% EtOAc in hexanes) gave methyl (3-bromo-1-(4-cyano-2-methoxybenzyl)-7-(((5-methylisoxazol-3-yl)methyl)amino)-1H-pyrazolo[4,3-d]pyrimidin-5-yl)carbamate (295 mg, 0.559 mmol, 32.7% yield) as a solid. LC-MS (ES, m/z): [M+H]⁺=527.1, 529.1.

¹H NMR (400 MHz, DMSO-d₆) δ 10.03 (s, 1H), 8.03 (t, J=5.7 Hz, 1H), 7.49 (d, J=1.3 Hz, 1H), 7.35 (dd, J=7.7, 1.3 Hz, 1H), 6.87 (d, J=7.9 Hz, 1H), 6.21 (d, J=0.9 Hz, 1H), 5.78 (s, 2H), 4.77 (d, J=5.7 Hz, 2H), 3.77 (s, 3H), 3.74-3.58 (m, 3H), 2.39-2.30 (m, 3H).

Step 2. To a stirred solution of methyl (3-bromo-1-(4-cyano-2-methoxybenzyl)-7-(((5-methylisoxazol-3-yl)methyl)amino)-1H-pyrazolo[4,3-d]pyrimidin-5-yl)carbamate (290 mg, 0.550 mmol) in ethanol (15 mL) was added 10% palladium on carbon (29 mg). The reaction mixture evacuated and purged with hydrogen six times, then the reaction was stirred at RT for 2 hours under a hydrogen atmosphere. The reaction mixture was evaporated to dryness, and the crude material was purified using flash chromatography (24 g SiO₂ column, 0 to 10% MeOH in DCM), giving methyl (3-bromo-1-(4-cyano-2-methoxybenzyl)-7-(((5-methylisoxazol-3-yl)methyl)amino)-1H-pyrazolo[4,3-d]pyrimidin-5-yl)carbamate (290 mg, 0.550 mmol) as a solid. LC-MS (ES, m/z): [M+H]⁺=449.1.

¹H NMR (400 MHz, DMSO-d₆) δ 10.37 (br s, 1H), 8.20 (br s, 1H), 8.01 (s, 1H), 7.50 (d, J=1.3 Hz, 1H), 7.32 (dd, J=7.8, 1.2 Hz, 1H), 6.71 (br d, J=7.5 Hz, 1H), 6.16 (s, 1H), 5.81 (s, 2H), 4.76 (br d, J=5.7 Hz, 2H), 3.80 (s, 3H), 3.69 (s, 3H), 2.35 (s, 3H).

Step 3. A microwave vial was charged with methyl (1-(4-cyano-2-methoxybenzyl)-7-(((5-methylisoxazol-3-yl)methyl)amino)-1H-pyrazolo[4,3-d]pyrimidin-5-yl)carbamate (70 mg, 0.156 mmol), THF (3 mL) and methylmagnesium bromide (0.260 mL, 0.780 mmol). The reaction was heated at 80° C. in the microwave for 20 min. Titanium(IV) isopropoxide (0.091 mL, 0.312 mmol) was added, followed by methylmagnesium bromide (0.260 mL, 0.780 mmol), and the reaction heated at 70° C. in the microwave for a further 20 min. More methylmagnesium bromide (0.260 mL, 0.780 mmol) was added, and the reaction heated in the microwave for a further 30 min at 70° C. The reaction mixture was quenched with NH₄Cl solution (10 mL) and extracted into EtOAc (3×5 mL). The combined organics were washed with brine (3×5 mL), dried (MgSO₄), filtered and concentrated. The residue was dissolved in dioxane (2 mL) and NaOH (0.937 mL, 4.68 mmol) added. The reaction was stirred at 80° C. for 2 hours, then allowed to cool. The reaction mixture was neutralized using 5N HCl, and then was evaporated to dryness, redissolved in DMF (2 ml), filtered and purified via preparative LC/MS with the following conditions: Column: XBridge C18, 200 mm×19 mm, 5-μm particles; Mobile Phase A: 5:95 acetonitrile: water with NH₄OAc; Mobile Phase B: 95:5 acetonitrile: water with NH₄OAc; Gradient: a 0-minute hold at 0% B, 0-40% B over 20 min, then a 0-minute hold at 100% B; Flow Rate: 20 mL/min; Column Temperature: 25 C. Fraction collection was triggered by MS signals. Fractions containing the desired product were combined and dried via centrifugal evaporation, giving Compound 103 (5.3 mg, 0.012 mmol, 7.65% yield).

### Example 4—Compound 104

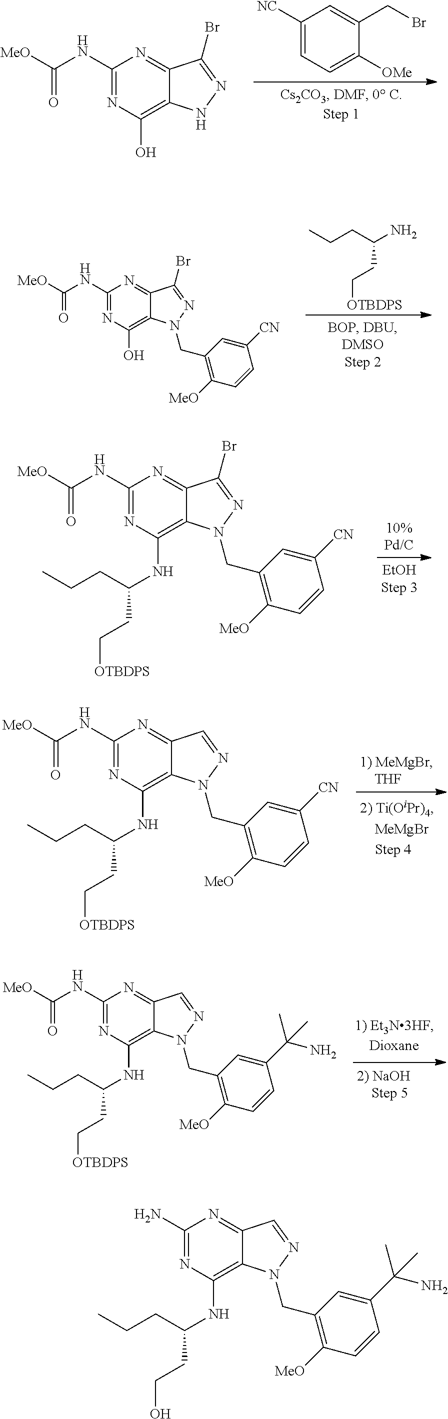

Step 1. To a stirred solution of methyl (3-bromo-7-hydroxy-1H-pyrazolo[4,3-d]pyrimidin-5-yl)carbamate (2 g, 6.94 mmol) in DMF (40 mL) was added Cs₂CO₃ (2.488 g, 7.64 mmol). After cooling in an ice bath, a solution of 3-(bromomethyl)-4-methoxybenzonitrile (1.570 g, 6.94 mmol) in DMF (10 mL) was added. The reaction was allowed to warm slowly to RT and stirred overnight. The reaction mixture was slowly poured into water (1 L) under stirring. The precipitate was filtered, washed with water (2×50 mL) and dried under vacuum to afford methyl (3-bromo-1-(5-cyano-2-methoxybenzyl)-7-hydroxy-1H-pyrazolo[4,3-d]pyrimidin-5-yl)carbamate (2.3 g, 5.31 mmol, 76% yield) as a gray solid.

LC-MS (ES, m/z): [M+H]=433.1.

¹H NMR (400 MHz, DMSO-d6) δ11.51 (brs, 2H), 7.83 (dd, J=8.6, 2.2 Hz, 1H), 7.35 (d, J=2.1 Hz, 1H), 7.23 (d, J=8.6 Hz, 1H), 5.70 (s, 2H), 3.88 (s, 3H), 3.76 (s, 3H).

Step 2. To a stirred solution of methyl (3-bromo-1-(5-cyano-2-methoxybenzyl)-7-hydroxy-1H-pyrazolo[4,3-d]pyrimidin-5-yl)carbamate (0.4 g, 0.923 mmol), (S)-1-((tert-butyldiphenylsilyl)oxy)hexan-3-amine (0.657 g, 1.847 mmol) and BOP (0.613 g, 1.385 mmol) in DMSO (9.23 ml) was added DBU (0.418 ml, 2.77 mmol). The reaction mixture was stirred at 60° C. for 1 h. The reaction mixture was poured into saturated NaHCO₃ solution (100 mL) and extracted into EtOAc (3×70 mL). The combined organic phases were washed with brine (4×40 mL), dried (MgSO₄), filtered and concentrated. The crude material was purified using flash chromatography (Isco, 40 g SiO₂ column, loaded in DCM, 0 to 50% EtOAc in hexanes over 30 min), giving methyl (S)-(3-bromo-7-((1-((tert-butyldiphenylsilyl)oxy)hexan-3-yl)amino)-1-(5-cyano-2-methoxybenzyl)-1H-pyrazolo[4,3-d]pyrimidin-5-yl)carbamate (0.34 g, 0.441 mmol, 47.8% yield) as a pale brown solid.

LC-MS (ES, m/z): [M+H]⁺=770.3.

¹H NMR (400 MHz, DMSO-d6) δ 9.58 (s, 1H), 7.55 (dd, J=8.6, 2.2 Hz, 1H), 7.37-7.31 (m, 2H), 7.30-7.25 (m, 2H), 7.20-7.10 (m, 4H), 7.06-6.99 (m, 3H), 6.94 (d, J=8.6 Hz, 1H), 6.48 (d, J=8.4 Hz, 1H), 5.61-5.32 (m, 2H), 4.44 (dq, J=11.5, 5.6, 4.1 Hz, 1H), 3.51 (s, 3H), 3.45 (t, J=6.5 Hz, 2H), 3.37 (s, 3H), 1.67 (m, 2H), 1.44-1.21 (m, 2H), 1.09-0.89 (m, 2H), 0.70 (s, 9H), 0.59 (t, J=7.3 Hz, 3H).

Step 3. Methyl (S)-(3-bromo-7-((1-((tert-butyldiphenylsilyl)oxy)hexan-3-yl)amino)-1-(5-cyano-2-methoxybenzyl)-1H-pyrazolo[4,3-d]pyrimidin-5-yl)carbamate (0.34 g, 0.441 mmol) was dissolved in EtOH (22.05 ml). 10% Pd on carbon (33 mg) was added, and the reaction mixture was evacuated and purged three times with hydrogen. The reaction was stirred under a hydrogen atmosphere for 1 hour. The reaction was filtered through CELITE™, washing with ethanol (50 mL) and the filtrate evaporated to give methyl (S)-(7-((1-((tert-butyldiphenylsilyl)oxy)hexan-3-yl)amino)-1-(5-cyano-2-methoxybenzyl)-1H-pyrazolo[4,3-d]pyrimidin-5-yl)carbamate (300 mg, 0.434 mmol, 98% yield) as a white solid. LC-MS (ES, m/z): [M+H]⁺=692.3.

¹H NMR (400 MHz, DMSO-d6) δ 8.08 (s, 1H), 7.92 (s, 1H), 7.85-7.76 (m, 1H), 7.66-7.58 (m, 1H), 7.52 (ddt, J=18.0, 6.9, 1.5 Hz, 4H), 7.46-7.32 (m, 5H), 7.32-7.11 (m, 4H), 5.95-5.63 (m, 2H), 4.61 (m, 1H), 3.82 (s, 3H), 3.77 (s, 3H), 3.73-3.61 (m, 2H), 1.94 (m, 2H), 1.59 (m, 2H), 1.31-1.11 (m, 3H), 0.92 (s, 9H), 0.82 (t, J=7.3 Hz, 3H).

Step 4. A microwave vial was charged with methyl (S)-(7-((1-((tert-butyldiphenyl-silyl)oxy)hexan-3-yl)amino)-1-(5-cyano-2-methoxybenzyl)-1H-pyrazolo[4,3-d]pyrimidin-5-yl)carbamate (50 mg, 0.072 mmol) and THF (2 mL). Methylmagnesium bromide (0.241 mL, 0.723 mmol) was added. The vial was capped and the reaction mixture was heated in a microwave oven for 20 min at 100° C. Titanium (IV) isopropoxide (0.042 mL, 0.145 mmol) was added, followed by methylmagnesium bromide (0.120 mL, 0.361 mmol). The reaction mixture was heated for a further 20 min in the microwave oven at 100° C. LCMS shows formation of product. The reaction mixture was diluted with EtOAc (50 mL) and quenched with saturated NH₄Cl solution (20 mL). Aqueous layer was extracted with EtOAc (2×10 mL). The combined organic phases were washed with brine (5 mL), dried (MgSO₄), filtered and concentrated, giving a yellow gum, which was further purified on Accq Prep 20×150 mm Xbridge column using acetonitrile/water (0.1% TFA). Fractions containing desired product were lyophilyzed to provide methyl (S)-(1-(5-(2-aminopropan-2-yl)-2-methoxybenzyl)-7-((1-((tert-butyldiphenylsilyl)-oxy)hexan-3-yl)amino)-1H-pyrazolo[4,3-d]pyrimidin-5-yl)carbamate (25 mg, 0.035 mmol, 47.8% yield).

LC-MS (ES, m/z): [M+H]⁺=724.4.

¹H NMR (400 MHz, DMSO-d6) δ 9.43 (brs, 1H), 8.33 (s, 2H), 7.81 (s, 1H), 7.59-7.47 (m, 3H), 7.42 (td, J=6.4, 2.9 Hz, 3H), 7.38-7.24 (m, 5H), 7.19 (t, J=7.4 Hz, 2H), 7.00 (d, J=8.8 Hz, 1H), 6.84 (d, J=2.6 Hz, 1H), 6.19 (d, J=8.4 Hz, 1H), 5.75-5.39 (m, 2H), 4.51 (q, J=7.1 Hz, 1H), 3.65 (s, 3H), 3.61 (t, J=1.6 Hz, 2H), 3.51 (s, 3H), 1.76 m, 2H), 1.55 (m, 2H), 1.43 (m, 2H), 1.37 (s, 3H), 1.35 (s, 3H), 1.07 (m, 2H), 0.86 (s, 9H), 0.72 (t, J=7.3 Hz, 3H).

Step 5. Methyl (S)-(1-(5-(2-aminopropan-2-yl)-2-methoxybenzyl)-7-((1-((tert-butyldiphenylsilyl)oxy)hexan-3-yl)amino)-1H-pyrazolo[4,3-d]pyrimidin-5-yl)carbamate (50 mg, 0.069 mmol) was dissolved in dioxane (2 mL). Triethylamine trihydrofluoride (0.056 mL, 0.345 mmol) was added and the reaction mixture was stirred at 70° C. for 1 h. NaOH (1.450 mL, 7.25 mmol) was then added. The reaction mixture was heated at 80° C. for a further 1 h. After cooling, the reaction mixture was neutralized with 5N HCl and evaporated to dryness. The residue was dissolved in DMF (2 mL), filtered and purified via preparative LC/MS with the following conditions: Column: XBridge C18, 200 mm×19 mm, 5-μm particles; Mobile Phase A: 5:95 acetonitrile: water with NH₄OAc; Mobile Phase B: 95:5 acetonitrile: water with NH₄OAc; Gradient: a 0-minute hold at 2% B, 2-42% B over 20 min, then a 0-min hold at 100% B; Flow Rate: 20 mL/min; Column Temperature: 25 C. Fraction collection was triggered by MS and UV signals. Fractions containing the desired product were combined and dried via centrifugal evaporation, giving Compound 104 (18.6 mg, 60% yield).

### Example 5—Compound 105

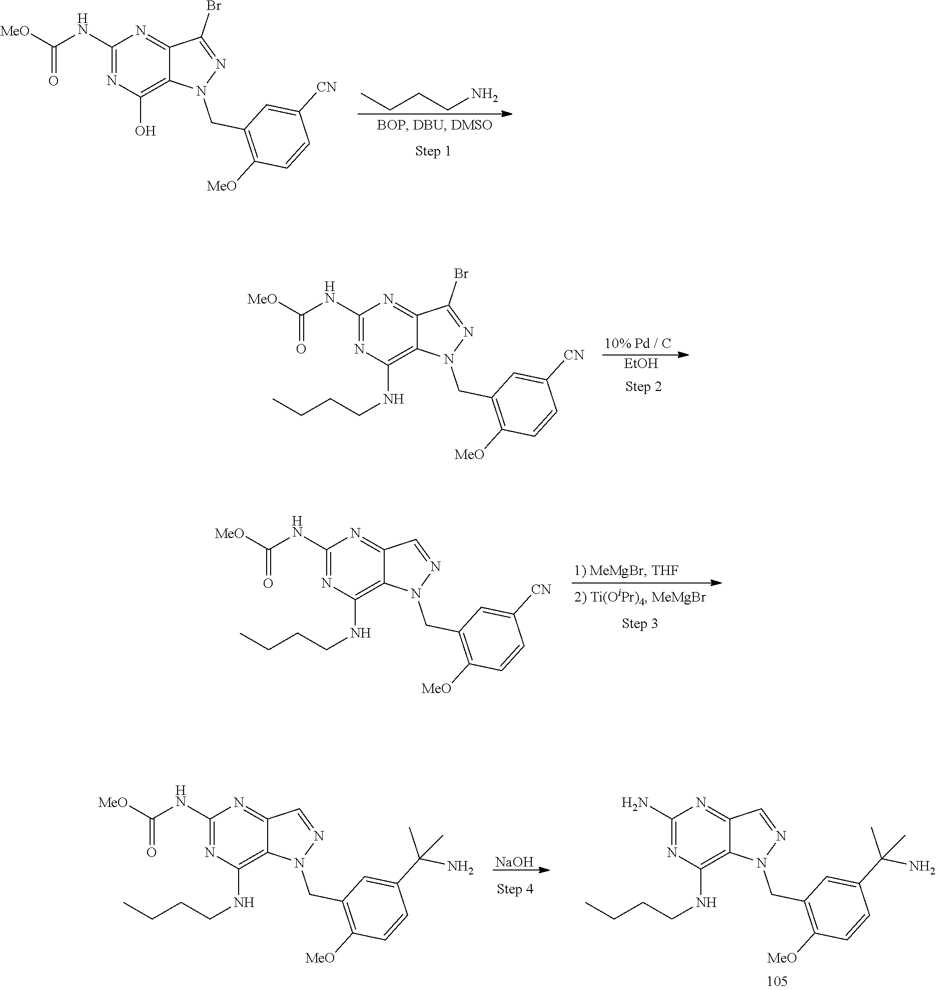

Step 1. To a stirred solution of methyl (3-bromo-1-(5-cyano-2-methoxybenzyl)-7-hydroxy-1H-pyrazolo[4,3-d]pyrimidin-5-yl)carbamate (500 mg, 1.154 mmol), butan-1-amine (0.172 mL, 1.731 mmol) and BOP (766 mg, 1.731 mmol) in DMSO (10 mL) was added DBU (0.522 mL, 3.46 mmol). The reaction was stirred at 60° C. for 20 min. The reaction mixture was poured into saturated NaHCO₃ solution (100 mL) and extracted with EtOAc (3×50 mL). The combined organic phases were washed with brine (4×40 mL), dried (MgSO₄), filtered and concentrated. The crude material was purified using flash chromatography (ISCO, 40 g SiO₂ column, loaded in DCM, 0 to 80% EtOAc in hexanes over 30 min), giving methyl (3-bromo-7-(butylamino)-1-(5-cyano-2-methoxybenzyl)-1H-pyrazolo[4,3-d]pyrimidin-5-yl)carbamate (0.4 g, 0.819 mmol, 71.0% yield) as a pale brown solid.

LC-MS (ES, m/z): [M+H]⁺=488.1.

¹H NMR (400 MHz, DMSO-d6) δ 9.87 (s, 1H), 7.84 (dd, J=8.6, 2.2 Hz, 1H), 7.41 (t, J=5.6 Hz, 1H), 7.35 (d, J=2.1 Hz, 1H), 7.22 (d, J=8.7 Hz, 1H), 5.71 (s, 2H), 3.82 (s, 3H), 3.64 (s, 3H), 3.61-3.48 (m, 2H), 1.61 (tt, J=7.7, 6.7 Hz, 2H), 1.38-1.21 (m, 2H), 0.89 (t, J=7.4 Hz, 3H).

Step 2. To a solution of methyl (3-bromo-7-(butylamino)-1-(5-cyano-2-methoxybenzyl)-1H-pyrazolo[4,3-d]pyrimidin-5-yl)carbamate (0.4 g, 0.819 mmol) in Ethanol (16.38 ml) was added 10% Pd/C (0.044 g, 0.041 mmol). The reaction mixture was evacuated and purged with hydrogen three times, then stirred overnight under a hydrogen atmosphere. The reaction mixture was filtered and the filtrate evaporated to dryness. The crude material was purified using flash chromatography (ISCO, 40 g SiO₂ column, solid loading, 0-20% MeOH in DCM over 20 min), giving methyl (7-(butylamino)-1-(5-cyano-2-methoxybenzyl)-1H-pyrazolo[4,3-d]pyrimidin-5-yl)carbamate (270 mg, 0.659 mmol, 81% yield) as a white solid.

LC-MS (ES, m/z): [M+H]⁺=410.2.

¹H NMR (400 MHz, DMSO-d6) δ 11.38 (s, 1H), 8.45 (s, 1H), 8.04 (s, 1H), 7.84 (dd, J=8.6, 2.2 Hz, 1H), 7.30 (d, J=2.1 Hz, 1H), 7.23 (d, J=8.6 Hz, 1H), 5.80 (s, 2H), 3.84 (s, 3H), 3.80 (s, 3H), 3.62 (dt, J=7.9, 5.9 Hz, 2H), 1.60 (p, J=7.3 Hz, 2H), 1.29 (h, J=7.3 Hz, 2H), 0.89 (t, J=7.4 Hz, 3H).

Step 3. A microwave vial was charged with methyl (7-(butylamino)-1-(5-cyano-2-methoxybenzyl)-1H-pyrazolo[4,3-d]pyrimidin-5-yl)carbamate (10 mg, 0.024 mmol) and THF (1 mL). Methylmagnesium bromide (0.144 mL, 0.488 mmol) was added. The vial was capped and the reaction heated in a microwave oven for 20 min at 90° C. Titanium (IV) isopropoxide (0.014 mL, 0.049 mmol) was added, followed by methylmagnesium bromide (0.072 mL, 10 eq). The reaction heated in the microwave oven for 20 min at 80° C. The reaction mixture was quenched with NH₄Cl solution (1 mL) and concentrated. The residue was dissolved in DMF (2 mL), filtered and purified on Accq Prep 30×150 mm Xbridge column. 50% acetonitrile/water (0.1% TFA) fractions collected at 15 min were lyophilyzed to provide methyl (1-(5-(2-aminopropan-2-yl)-2-methoxybenzyl)-7-(butylamino)-1H-pyrazolo[4,3-d]pyrimidin-5-yl)carbamate, TFA salt (4 mg, 7.20 μmol, 29.5% yield) as white solid.

LC-MS (ES, m/z): [M+H]⁺=442.5.

Step 4. Methyl (1-(5-(2-aminopropan-2-yl)-2-methoxybenzyl)-7-(butylamino)-1H-pyrazolo[4,3-d]pyrimidin-5-yl)carbamate (9 mg, 0.020 mmol) was dissolved in dioxane (2 mL). NaOH (0.041 mL, 0.408 mmol) was added and the reaction stirred at 80° C. for 1 h. After cooling, the reaction mixture was neutralized with 6N HCl and evaporated to dryness. The residue was dissolved in DMF (2 ml), filtered and purified via preparative LC/MS with the following conditions: Column: XBridge C18, 200 mm×19 mm, 5-μm particles; Mobile Phase A: 5:95 acetonitrile: water with NH₄OAc; Mobile Phase B: 95:5 acetonitrile: water with NH₄OAc; Gradient: a 0-minute hold at 2% B, 2-42% B over 20 min, then a 0-minute hold at 100% B; Flow Rate: 20 ml/min; Column Temperature: 25 C. Fraction collection was triggered by MS and UV signals. Fractions containing the desired product were combined and dried via centrifugal evaporation, giving Compound 105 (3.4 mg, 8.10 μmol, 39.8% yield).

### Example 6—Compound 106

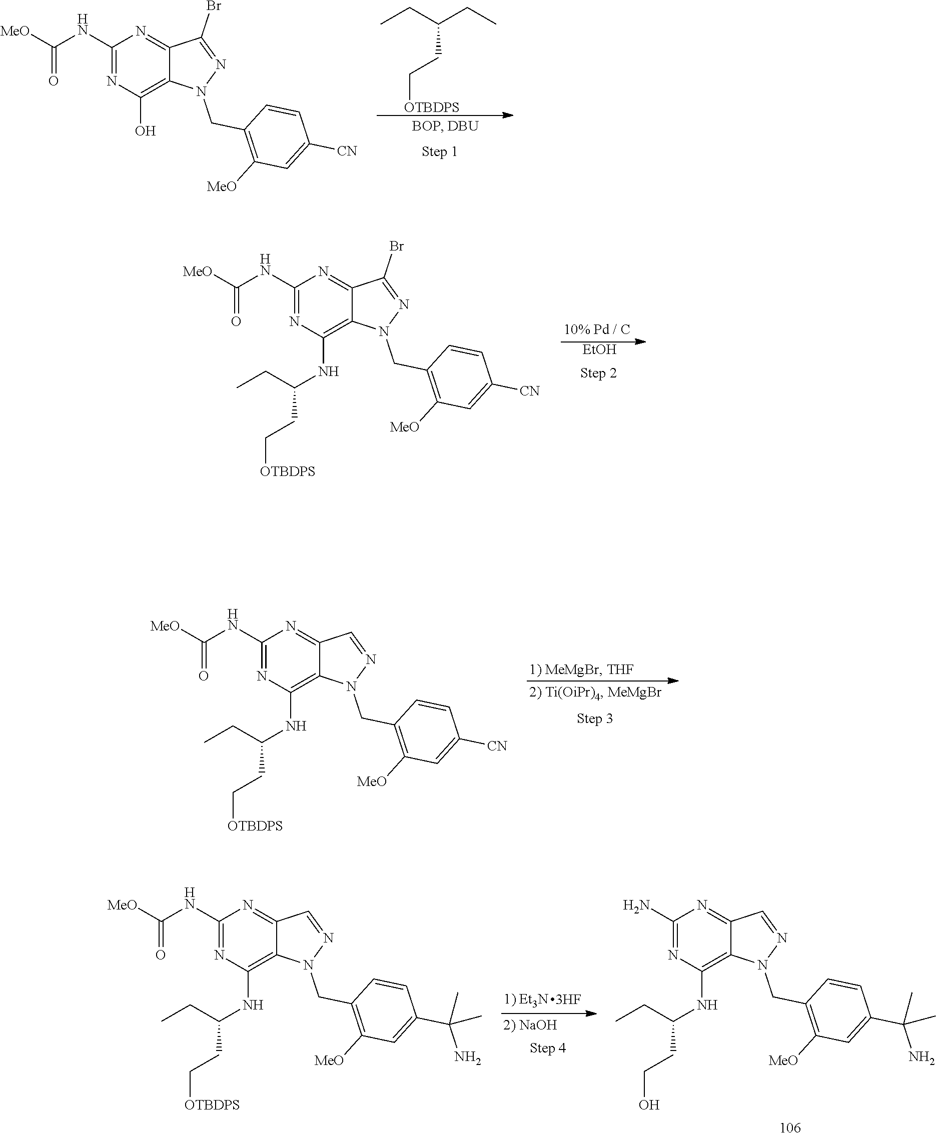

Step 1. To a stirred solution of methyl (3-bromo-1-(4-cyano-2-methoxybenzyl)-7-hydroxy-1H-pyrazolo[4,3-d]pyrimidin-5-yl)carbamate (1 g, 2.31 mmol), (S)-1-((tert-butyldiphenylsilyl)oxy)pentan-3-amine (1.18 g, 3.46 mmol) and BOP (1.53 g, 3.46 mmol) in DMSO (20 mL) was added DBU (1.04 mL, 6.92 mmol). The reaction was stirred at 60° C. for 1 hour. The reaction mixture was poured into saturated NaHCO₃ solution (100 mL) and extracted into EtOAc (3×70 mL). The combined organics were washed with brine (4×40 ml), dried (MgSO₄), filtered and concentrated. The crude material was purified using flash chromatography (80 g SiO₂ column, 0 to 50% EtOAc in hexanes), giving methyl (S)-(3-bromo-7-((1-((tert-butyldiphenylsilyl)oxy)pentan-3-yl)amino)-1-(4-cyano-2-methoxybenzyl)-1H-pyrazolo[4,3-d]pyrimidin-5-yl)carbamate (320 mg, 0.42 mmol, 18.3% yield) as a gum.

LC-MS (ES, m/z): [M+H]⁺=756.2, 758.2.

Step 2. To a solution of methyl (S)-(3-bromo-7-((1-((tert-butyldiphenylsilyl)-oxy)pentan-3-yl)amino)-1-(4-cyano-2-methoxybenzyl)-1H-pyrazolo[4,3-d]pyrimidin-5-yl)carbamate (320 mg, 0.42 mmol) in ethanol (5 mL) was added 10% palladium on carbon (32 mg). The reaction mixture was evacuated and purged with hydrogen six times, then stirred under a hydrogen atmosphere for 2 hours. The reaction mixture was filtered and concentrated. The crude material was purified using flash chromatography (40 g SiO₂ column, 0 to 100% EtOAc in hexanes, then 0 to 10% MeOH in DCM), giving methyl (S)-(7-((1-((tert-butyldiphenyl-silyl)oxy)pentan-3-yl)amino)-1-(4-cyano-2-methoxybenzyl)-1H-pyrazolo[4,3-d]pyrimidin-5-yl)carbamate (120 mg, 0.18 mmol, 41.9% yield) as a solid.

LC-MS (ES, m/z): [M+H]⁺=678.3.

¹H NMR (400 MHz, DMSO-d₆) δ 9.58 (s, 1H), 7.94 (s, 1H), 7.56-7.51 (m, 2H), 7.51-7.33 (m, 7H), 7.24-7.18 (m, 2H), 6.98 (dd, J=7.7, 1.3 Hz, 1H), 6.42-6.31 (m, 2H), 5.85-5.74 (m, 2H), 4.54-4.44 (m, 1H), 3.85-3.76 (m, 3H), 3.66-3.55 (m, 3H), 1.87-1.71 (m, 2H), 1.55 (quin, J=7.2 Hz, 2H), 0.91 (s, 9H), 0.75 (t, J=7.4 Hz, 3H).

Step 3. A microwave vial was charged with methyl (S)-(7-((1-((tert-butyldiphenylsilyl)oxy)pentan-3-yl)amino)-1-(4-cyano-2-methoxybenzyl)-1H-pyrazolo[4,3-d]pyrimidin-5-yl)carbamate (115 mg, 0.17 mmol) and THF (10 mL). Methylmagnesium bromide (0.28 mL, 0.85 mmol) solution was added, the vial was capped and the reaction heated in the microwave for 20 min at 80° C. Titanium(IV) isopropoxide (0.1 mL, 0.34 mmol) was added, followed by more methylmagnesium bromide (0.28 mL, 0.85 mmol) solution. The reaction was heated for a further 20 min in the microwave at 80° C. The reaction mixture was quenched with saturated NH₄Cl solution (10 mL) and extracted into EtOAc (3×5 mL). The combined organics were washed with brine (5 mL), dried (MgSO₄), filtered and concentrated, giving methyl (S)-(1-(4-(2-aminopropan-2-yl)-2-methoxybenzyl)-7-((1-((tert-butyldiphenylsilyl)oxy)pentan-3-yl)amino)-1H-pyrazolo[4,3-d]pyrimidin-5-yl)carbamate (105 mg, 60% purity, 0.053 mmol, 52.3% yield) as a gum.

LC-MS (ES, m/z): [M+H]⁺=710.6.

Step 4. Methyl (S)-(1-(4-(2-aminopropan-2-yl)-2-methoxybenzyl)-7-((1-((tert-butyldiphenylsilyl)oxy)pentan-3-yl)amino)-1H-pyrazolo[4,3-d]pyrimidin-5-yl)carbamate (105 mg, 0.148 mmol) was dissolved in dioxane (4 mL). Triethylamine trihydrofluoride (0.120 mL, 0.739 mmol) was added and the reaction stirred at 70° C. for 2 hours. 5N NaOH solution (1.479 mL, 7.39 mmol) was then added, and the reaction heated at 70° C. for a further 2 hours. After cooling, the reaction mixture was neutralized with 5N HCl and evaporated to dryness. The crude material was dissolved in DMF (2 ml), filtered and purified via preparative LC/MS with the following conditions: Column: XBridge C18, 200 mm×19 mm, 5-μm particles; Mobile Phase A: 5:95 acetonitrile: water with NH₄OAc; Mobile Phase B: 95:5 acetonitrile: water with NH₄OAc; Gradient: a 0-minute hold at 0% B, 0-40% B over 20 min, then a 0-minute hold at 100% B; Flow Rate: 20 mL/min; Column Temperature: 25 C. Fraction collection was triggered by MS and UV signals. Fractions containing the desired product were combined and dried via centrifugal evaporation, giving Compound 106 (8.4 mg, 0.020 mmol, 13.5% yield).

### Example 7—Compound 107, ditrifluoroacetate

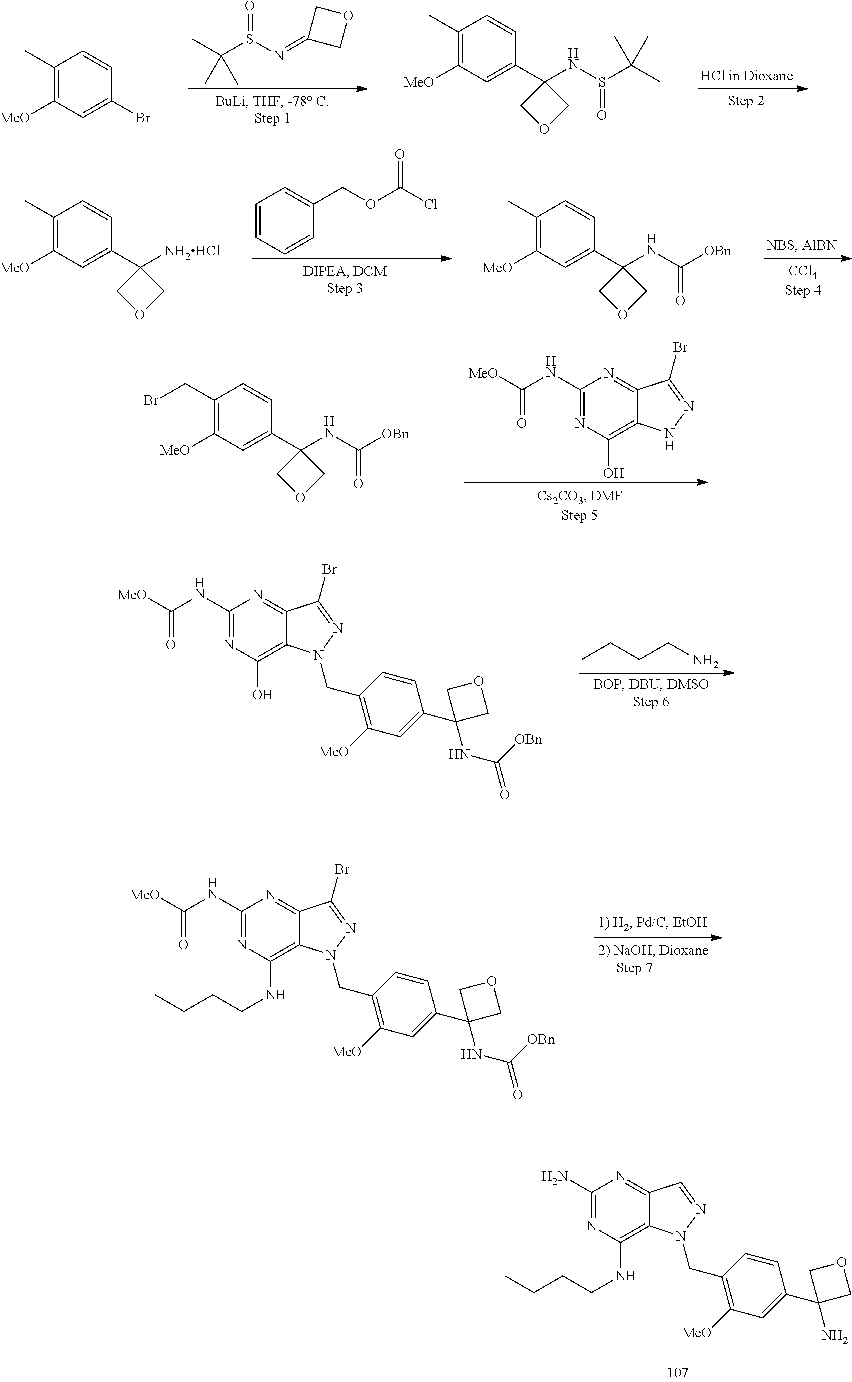

Step 1. A stirred solution of 4-bromo-2-methoxy-1-methylbenzene (2 g, 9.95 mmol) in tetrahydrofuran (90 mL) was cooled to −78° C. n-butyllithium (9.33 mL, 14.92 mmol) was added, and the reaction stirred at −78° C. for 1 hour. A solution of 2-methyl-N-(oxetan-3-ylidene)propane-2-sulfinamide (1.918 g, 10.94 mmol) in tetrahydrofuran (10 mL) was added, and the reaction allowed to warm slowly to RT and stirred for 2 hours. The reaction mixture was poured into saturated NH₄Cl solution (100 mL) and extracted into EtOAc (3×50 mL). The combined organics were washed with brine (3×40 mL), dried (MgSO₄), filtered and concentrated. The crude material was purified using flash chromatography (80 g SiO₂ column, 0 to 75% EtOAc in hexanes), giving N-(3-(3-methoxy-4-methylphenyl)oxetan-3-yl)-2-methylpropane-2-sulfinamide (937 mg, 3.15 mmol, 31.7% yield) as a gum.

LC-MS (ES, m/z): [M+H]⁺=298.2.

¹H NMR (400 MHz, DMSO-d₆) δ 7.16 (d, J=7.7 Hz, 1H), 7.00 (d, J=7.6 Hz, 1H), 6.98 (s, 1H), 6.23 (s, 1H), 5.00 (t, J=6.2 Hz, 2H), 4.89 (d, J=6.6 Hz, 1H), 4.67 (d, J=6.2 Hz, 1H), 3.79 (s, 3H), 2.15 (s, 3H), 1.20-1.07 (m, 9H).

Step 2. To a stirred solution of N-(3-(3-methoxy-4-methylphenyl)oxetan-3-yl)-2-methylpropane-2-sulfinamide (700 mg, 2.354 mmol) in dioxane (25 mL) was added 4N HCl in dioxane (1.177 mL, 4.71 mmol). The reaction was stirred at RT for 20 min. The product was filtered off, washing with diethyl ether (100 mL) to give 3-(3-methoxy-4-methylphenyl)oxetan-3-amine hydrochloride (501 mg, 2.181 mmol, 93% yield) as a solid.

LC-MS (ES, m/z): [M+H]⁺=194.2.

¹H NMR (400 MHz, DMSO-d₆) δ 9.24 (br s, 3H), 7.34-7.10 (m, 2H), 7.01 (br s, 1H), 4.96 (br s, 4H), 3.84 (br s, 3H), 2.17 (br s, 3H).

Step 3. A solution of 3-(3-methoxy-4-methylphenyl)oxetan-3-amine hydrochloride (500 mg, 2.177 mmol) and DIPEA (0.950 mL, 5.44 mmol) in DCM (25 mL) was cooled in an ice bath. Benzyl chloroformate (0.340 mL, 2.394 mmol) was added, and the reaction allowed to warm to RT and stirred for 1 hour. The reaction mixture was poured into saturated NaHCO₃ solution (100 mL) and extracted into DCM (3×50 ml). The combined organics were washed with brine (3×40 ml), dried (MgSO₄), filtered and concentrated. The crude material was purified using flash chromatography (40 g SiO₂ column, 0 to 50% EtOAc in hexanes), giving benzyl (3-(3-methoxy-4-methylphenyl)oxetan-3-yl)carbamate (475 mg, 1.451 mmol, 66.7% yield) as an oil.

LC-MS (ES, m/z): [M+H]⁺=328.2.

¹H NMR (400 MHz, DMSO-d₆) δ 8.54-8.36 (m, 1H), 7.36 (br s, 5H), 7.12 (d, J=7.9 Hz, 1H), 7.00-6.91 (m, 2H), 5.02 (br s, 2H), 4.82 (d, J=6.6 Hz, 2H), 4.72 (d, J=6.4 Hz, 2H), 3.73 (s, 3H), 2.13 (s, 3H).

Step 4. A solution of benzyl (3-(3-methoxy-4-methylphenyl)oxetan-3-yl)carbamate (470 mg, 1.436 mmol), NBS (268 mg, 1.507 mmol) and AIBN (47.1 mg, 0.287 mmol) in CCl₄ (15 mL) was heated to 75° C. and maintained at this temperature for 1 hour. After cooling, the reaction mixture was evaporated to dryness and the crude material purified using flash chromatography (40 g SiO₂ column, 0 to 40% EtOAc in hexanes), giving benzyl (3-(4-(bromomethyl)-3-methoxyphenyl)oxetan-3-yl)carbamate (175 mg, 0.431 mmol, 30.0% yield) as an oil.

LC-MS (ES, m/z): [M+H]⁺=406.2, 408.1.

¹H NMR (400 MHz, DMSO-d₆) δ 8.57-8.45 (m, 1H), 7.47-7.29 (m, 6H), 7.09-6.98 (m, 2H), 5.03 (br s, 2H), 4.82 (d, J=6.8 Hz, 2H), 4.79-4.71 (m, 2H), 4.68-4.62 (m, 2H), 3.86-3.79 (m, 3H).

Step 5. To a stirred solution of methyl (3-bromo-7-hydroxy-1H-pyrazolo[4,3-d]pyrimidin-5-yl)carbamate (120 mg, 0.417 mmol) in DMF (2 mL) at 0° C. was added Cs₂CO₃ (204 mg, 0.625 mmol) followed by a solution of benzyl (3-(4-(bromomethyl)-3-methoxyphenyl)oxetan-3-yl)carbamate (169 mg, 0.417 mmol) in DMF (1 mL). The reaction was allowed to warm to RT and stirred overnight. The reaction mixture was poured into saturated NaHCO₃ solution (100 mL) and extracted into EtOAc (3×50 mL). The combined organics were washed with brine (3×40 mL), dried (MgSO₄), filtered and concentrated, giving methyl (1-(4-(3-(((benzyloxy)carbonyl)amino)oxetan-3-yl)-2-methoxybenzyl)-3-bromo-7-hydroxy-1H-pyrazolo[4,3-d]pyrimidin-5-yl)carbamate (220 mg, 0.251 mmol, 60.3% yield, 70% purity) as a solid.

LC-MS (ES, m/z): [M+H]⁺=613.2, 615.2.

Step 6. A solution of methyl (1-(4-(3-(((benzyloxy)carbonyl)amino)oxetan-3-yl)-2-methoxybenzyl)-3-bromo-7-hydroxy-1H-pyrazolo[4,3-d]pyrimidin-5-yl)carbamate (220 mg, 0.251 mmol, 70% purity), butan-1-amine (52.5 mg, 0.717 mmol), BOP (238 mg, 0.538 mmol) and DBU (0.162 mL, 1.076 mmol) in DMSO (4 mL) was heated to 60° C. for 20 min, then cooled to RT. The reaction mixture was poured into saturated NaHCO₃ solution (100 mL) and extracted into EtOAc (3×50 mL). The combined organics were washed with brine (4×40 ml), dried (MgSO₄), filtered and concentrated, giving methyl (1-(4-(3-(((benzyloxy)carbonyl)amino)oxetan-3-yl)-2-methoxybenzyl)-3-bromo-7-(butylamino)-1H-pyrazolo[4,3-d]pyrimidin-5-yl)carbamate (245 mg, 0.220 mmol, 87.6% yield, 60% purity) as an oil.

LC-MS (ES, m/z): [M+H]⁺=668.3, 670.3.

Step 7. To a solution of methyl (1-(4-(3-(((benzyloxy)carbonyl)amino)oxetan-3-yl)-2-methoxybenzyl)-3-bromo-7-(butylamino)-1H-pyrazolo[4,3-d]pyrimidin-5-yl)carbamate (240 mg, 0.215 mmol) in EtOH (10 mL) was added 10% palladium on carbon (24 mg). The reaction mixture was evacuated and purged six times with H₂, then stirred under a H₂ atmosphere for 24 h. The reaction mixture was filtered and evaporated to dryness. The residue was dissolved in dioxane (4 mL) and NaOH (1.077 mL, 5.38 mmol) was added. The reaction mixture was stirred at 80° C. for 2 h, cooled, neutralized using 5N HCl and evaporated to dryness. The residue was dissolved in DMF (2 ml), filtered and purified via preparative LC/MS with the following conditions: Column: XBridge C18, 200 mm×19 mm, 5-μm particles; Mobile Phase A: 5:95 acetonitrile: water with NH₄OAc; Mobile Phase B: 95:5 acetonitrile: water with NH₄OAc; Gradient: a 0-minute hold at 10% B, 10-50% B over 20 min, then a 0-minute hold at 100% B; Flow Rate: 20 mL/min; Column Temperature: 25° C. Fraction collection was triggered by MS and UV signals. Fractions containing the product were combined and dried via centrifugal evaporation. The material was further purified via preparative LC/MS with the following conditions: Column: XBridge Phenyl, 200 mm×19 mm, 5-μm particles; Mobile Phase A: 5:95 acetonitrile: water with 0.05% TFA; Mobile Phase B: 95:5 acetonitrile: water with 0.05% TFA; Gradient: a 0-minute hold at 5% B, 5-55% B over 20 min, then a 0-minute hold at 100% B; Flow Rate: 20 mL/min; Column Temperature: 25 C. Fraction collection was triggered by MS and UV signals. Fractions containing the desired product were combined and dried via centrifugal evaporation, giving Compound 107 ditrifluoracetate (22.2 mg, 0.032 mmol, 14.94% yield).

### Example 8—Compound 108

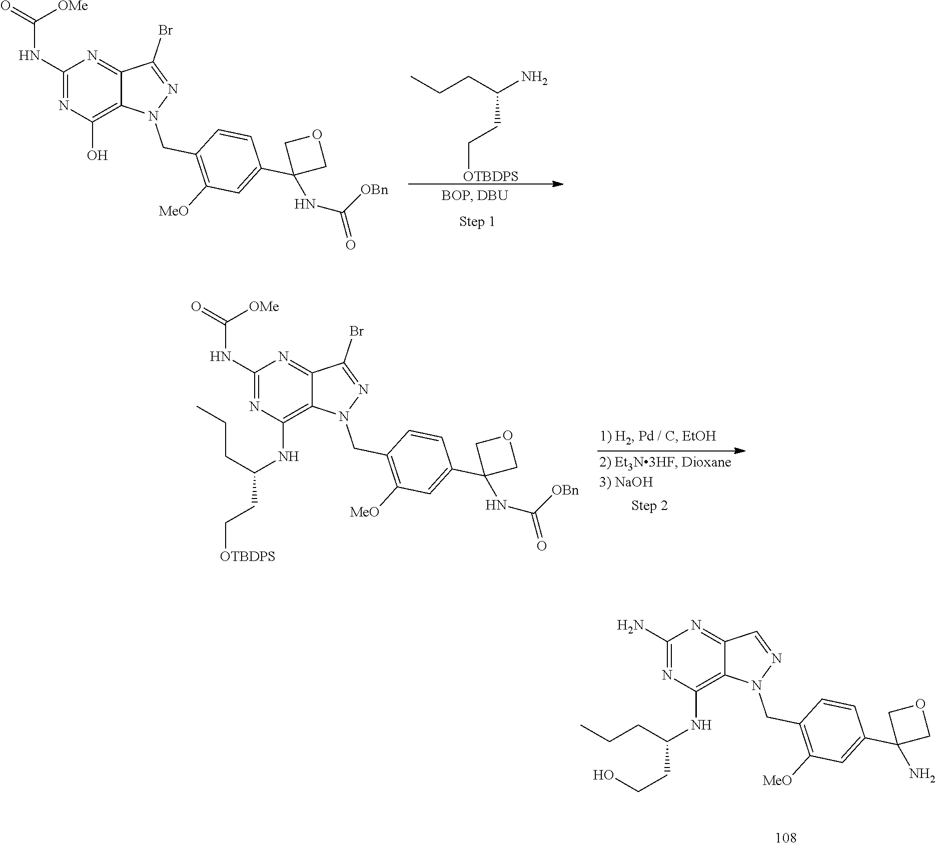

Step 1. To a stirred solution of methyl (1-(4-(3-(((benzyloxy)carbonyl)amino)oxetan-3-yl)-2-methoxybenzyl)-3-bromo-7-hydroxy-1H-pyrazolo[4,3-d]pyrimidin-5-yl)carbamate (150 mg, 0.245 mmol), BOP (162 mg, 0.367 mmol) and DBU (0.111 mL, 0.734 mmol) in DMSO (2 mL) was added a solution of (S)-1-((tert-butyldiphenylsilyl)oxy)hexan-3-amine (130 mg, 0.367 mmol) in DMSO (2 mL). The reaction was stirred at 60° C. for 1 hour. The reaction mixture was quenched with NaHCO₃ solution (10 mL) and extracted into EtOAc (3×8 mL). The combined organic phases were washed with brine (4×5 mL), dried (MgSO₄), filtered and concentrated. The crude material was purified using flash chromatography (24 g SiO₂ column, 0 to 60% EtOAc in hexanes), giving methyl (S)-(1-(4-(3-(((benzyloxy)carbonyl)amino)oxetan-3-yl)-2-methoxyben-zyl)-3-bromo-7-((1-((tert-butyldiphenylsilyl)oxy)hexan-3-yl)amino)-1H-pyrazolo[4,3-d]pyrimidin-5-yl)carbamate (79 mg, 0.083 mmol, 34.0% yield) as a gum.

LC-MS (ES, m/z): [M+H]⁺=950.5, 952.5.

Step 2. Methyl (S)-(1-(4-(3-(((benzyloxy)carbonyl)amino)oxetan-3-yl)-2-methoxy-benzyl)-3-bromo-7-((1-((tert-butyldiphenylsilyl)oxy)hexan-3-yl)amino)-1H-pyrazolo[4,3-d]pyri-midin-5-yl)carbamate (79 mg, 0.083 mmol) was dissolved in ethanol (10 mL). 10% palladium on carbon (8 mg) was added. The reaction mixture was evacuated and purged with H₂ six times, then stirred under an H₂ atmosphere for 16 h. The reaction mixture was filtered and evaporated to dryness. The residue was dissolved in dioxane (3 mL) and triethylamine trihydrofluoride (0.135 mL, 0.831 mmol) was added. The reaction was stirred at 60° C. for 2 h. 5N NaOH (0.665 mL, 3.32 mmol) was added, and the reaction stirred for a further 2 hours at 80° C. After cooling, the reaction was neutralized with 5N HCl and evaporated to dryness. The crude material was dissolved in DMF (2 ml), filtered and purified via preparative LC/MS with the following conditions: Column: XBridge C18, 200 mm×19 mm, 5-μm particles; Mobile Phase A: 5:95 acetonitrile: water with NH₄OAc; Mobile Phase B: 95:5 acetonitrile: water with NH₄OAc; Gradient: a 0-minute hold at 3% B, 3-43% B over 20 min, then a 0-minute hold at 100% B; Flow Rate: 20 mL/min; Column Temperature: 25 C. Fraction collection was triggered by MS and UV signals. Fractions containing the desired product were combined and dried via centrifugal evaporation, giving Compound 108 (15.0 mg, 0.034 mmol, 40.9% yield).

### Example 9—Compound 110

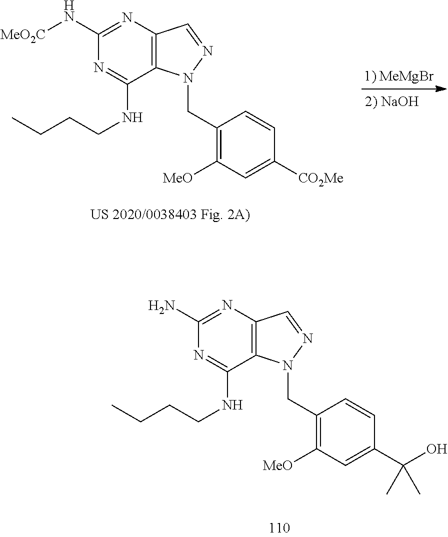 [SMILES: CCCCNc1nc(NC(=O)OC)nc2cnn(Cc3ccc(C(=O)OC)cc3OC)c12 ; CCCCNc1nc(N)nc2cnn(Cc3ccc(C(C)(C)O)cc3OC)c12]

To a stirred solution of methyl 4-((7-(butylamino)-5-((methoxycarbonyl)amino)-1H-pyrazolo[4,3-d]pyrimidin-1-yl)methyl)-3-methoxybenzoate (40 mg, 0.090 mmol) in THF (1 mL) in a 4 mL scintillation vial was added methylmagnesium bromide (0.075 mL, 0.226 mmol). The reaction was stirred at RT for 30 min and quenched with water (1 mL), followed by stirring for 10 min and evaporation to dryness. The crude material was dissolved in dioxane (1 mL) and NaOH (0.271 mL, 1.356 mmol) was added. The reaction mixture was heated to 80° C. and maintained at this temperature overnight. After cooling, the reaction mixture was neutralized with 5N HCl (271 uL) and evaporated to dryness. The residue was dissolved in DMF (2 ml), filtered and purified via preparative LC/MS with the following conditions: Column: XBridge C18, 200 mm×19 mm, 5-μm particles; Mobile Phase A: 5:95 acetonitrile: water with NH₄OAc; Mobile Phase B: 95:5 acetonitrile: water with NH₄OAc; Gradient: a 0-minute hold at 14% B, 14-54% B over 20 min, then a 0-minute hold at 100% B; Flow Rate: 20 mL/min; Column Temperature: 25° C. Fraction collection was triggered by MS signals. Fractions containing the desired product were combined and dried via centrifugal evaporation, giving Compound 110 (4.3 mg, 12% yield).

Compound 111 was analogously prepared.

### Example 10—Compound 112

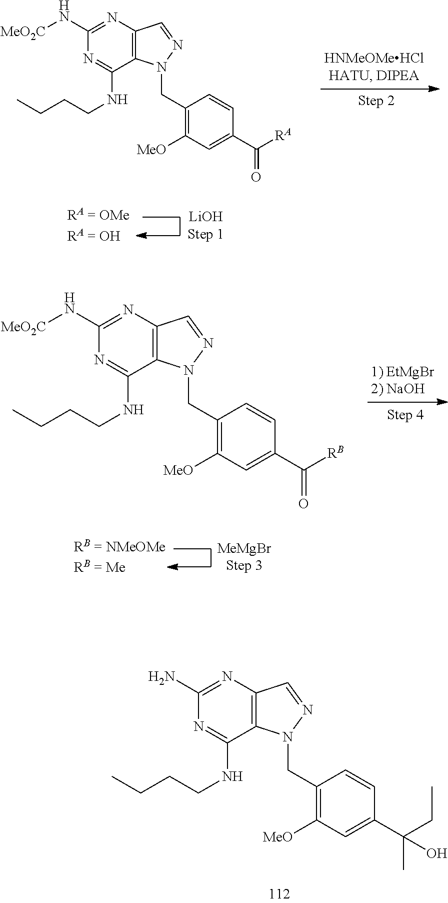

Step 1. To a stirred solution of methyl 4-((7-(butylamino)-5-((methoxycarbonyl)-amino)-1H-pyrazolo[4,3-d]pyrimidin-1-yl)methyl)-3-methoxybenzoate (500 mg, 1.130 mmol) in THF (6 mL) was added lithium hydroxide (3.39 mL, 3.39 mmol). The reaction mixture was stirred overnight at 30° C. As the reaction was not complete, more lithium hydroxide (3.39 mL, 3.39 mmol) was added, and the reaction mixture was stirred for a further 24 hours at 30° C. The reaction mixture was evaporated to dryness and purified using reverse-phase flash chromatography (50 g C₁₈ column, loaded in DMSO/water/MeCN, 0 to 70% MeCN in water containing 0.05% formic acid), giving 4-((7-(butylamino)-5-((methoxycarbonyl)amino)-1H-pyrazolo[4,3-d]pyrimidin-1-yl)methyl)-3-methoxybenzoic acid (262 mg, 54% yield) as a solid.

¹H NMR (400 MHz, DMSO-d₆) δ 13.00 (br s, 1H), 9.64 (s, 1H), 7.90 (s, 1H), 7.54-7.49 (m, 1H), 7.43 (dd, J=7.8, 1.2 Hz, 1H), 7.03 (br t, J=5.4 Hz, 1H), 6.53 (d, J=7.9 Hz, 1H), 5.80 (s, 2H), 3.89 (s, 3H), 3.63 (s, 3H), 3.55-3.39 (m, 3H), 1.58-1.44 (m, 2H), 1.18 (sxt, J=7.4 Hz, 2H), 0.83 (t, J=7.4 Hz, 3H).

LC/MS [M+H]⁺ 429.18.

Step 2. To a stirred solution of 4-((7-(butylamino)-5-((methoxycarbonyl)amino)-1H-pyrazolo[4,3-d]pyrimidin-1-yl)methyl)-3-methoxybenzoic acid (262 mg, 0.612 mmol), HATU (256 mg, 0.673 mmol) and N,O-dimethylhydroxylamine hydrochloride (84 mg, 0.856 mmol) was added DIPEA (0.235 mL, 1.345 mmol). The reaction was stirred for 1 hour at RT. The reaction mixture was poured into saturated NaHCO₃ solution (30 mL) and extracted with EtOAc (3×30 mL). The combined organic phases were washed with brine (4×20 mL), dried (MgSO₄), filtered and concentrated, giving methyl (7-(butylamino)-1-(2-methoxy-4-(methoxy(methyl)carbamoyl)-benzyl)-1H-pyrazolo[4,3-d]pyrimidin-5-yl)carbamate (280 mg, 97% yield) as a solid.

LC/MS [M+H]⁺472.22.

Step 3. To a stirred solution of methyl (7-(butylamino)-1-(2-methoxy-4-(methoxy-(methyl)carbamoyl)benzyl)-1H-pyrazolo[4,3-d]pyrimidin-5-yl)carbamate (250 mg, 0.530 mmol) in THF (4 mL) was added methylmagnesium bromide (0.884 mL, 2.65 mmol). The reaction was stirred at RT for 30 min. The reaction mixture was poured into saturated NH₄Cl solution (50 mL) and extracted with EtOAc (3×30 mL). The combined organic phases were washed with brine (3×30 ml), dried (MgSO₄), filtered and concentrated. Flash chromatography (40 g SiO₂ column, loaded in DCM, 0 to 10% MeOH in DCM) gave methyl (1-(4-acetyl-2-methoxybenzyl)-7-(butylamino)-1H-pyrazolo[4,3-d]pyrimidin-5-yl)carbamate (140 mg, 61%) as a solid.

¹H NMR (400 MHz, DMSO-d₆) δ 9.64 (s, 1H), 7.90 (s, 1H), 7.49 (s, 1H), 7.48 (d, J=7.5 Hz, 1H), 7.06 (br t, J=5.4 Hz, 1H), 6.55 (d, J=7.9 Hz, 1H), 5.80 (s, 2H), 3.90 (s, 3H), 3.63 (s, 3H), 3.52-3.43 (m, 2H), 3.31 (s, 3H), 1.53 (quin, J=7.3 Hz, 2H), 1.19 (sxt, J=7.4 Hz, 2H), 0.83 (t, J=7.4 Hz, 3H).

LC/MS [M+H]⁺ 425.1.

Step 4. Methyl (1-(4-acetyl-2-methoxybenzyl)-7-(butylamino)-1H-pyrazolo[4,3-d]pyrimidin-5-yl)carbamate (25 mg, 0.059 mmol) was dissolved in THF (5 mL). EtMgBr (39.1 mg, 0.293 mmol) was added. The reaction mixture was stirred for 30 min at RT, quenched with MeOH (1 ml), and evaporated to dryness. The residue was dissolved in dioxane (3 mL). NaOH (0.234 mL, 1.172 mmol) was added, and the reaction stirred at 80° C. for 4 h. After cooling, the reaction mixture was neutralized with 1N HCl, then evaporated to dryness. The residue was dissolved in DMF (2 ml), filtered and purified via preparative LC/MS with the following conditions: Column: XBridge C18, 200 mm×19 mm, 5-μm particles; Mobile Phase A: 5:95 acetonitrile:water with 10-mM NH₄OAc; Mobile Phase B: 95:5 acetonitrile:water with 10 mM NH₄OAc; Gradient: a 0-minute hold at 16% B, 16-56% B over 20 min, then a 0-minute hold at 100% B; Flow Rate: 20 mL/min; Column Temperature: 25 C. Fraction collection was triggered by MS and UV signals. Fractions containing Compound 112 were combined and dried via centrifugal evaporation (3.9 mg, 17% yield).

Compound 113 was analogously prepared. See Table A for analytical data.

### Example 11—Compound 114

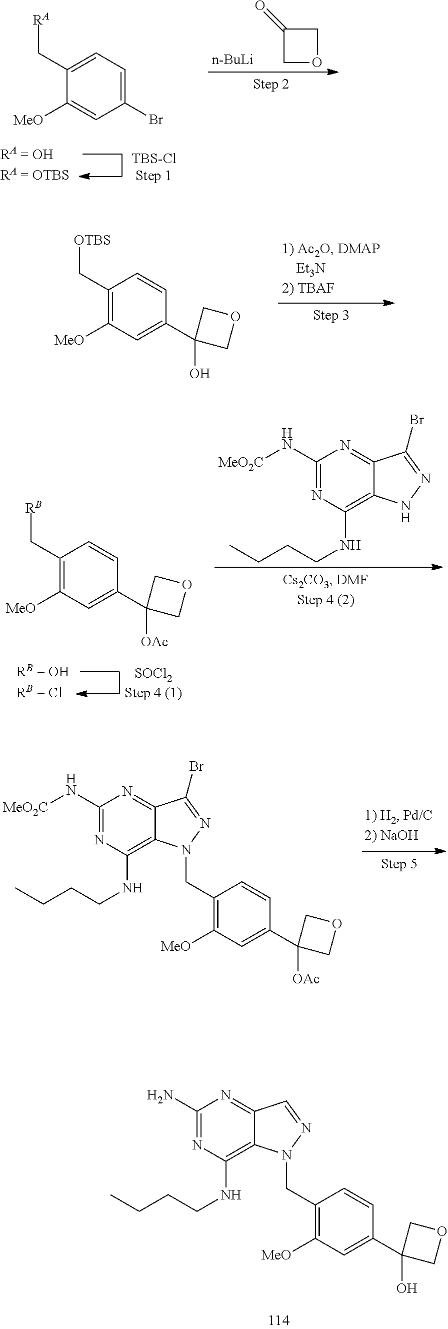

Step 1. A solution of (4-bromo-2-methoxyphenyl)methanol (5 g, 23.03 mmol), TBS-Cl (4.17 g, 27.6 mmol) and imidazole (2.195 g, 32.2 mmol) in DMF (50 mL) was stirred overnight at RT. The reaction mixture was poured into saturated NaHCO₃ solution (100 mL) and extracted with EtOAc (3×70 mL). The combined organic phases were washed with brine (4×50 ml), dried (MgSO₄), filtered and concentrated. Flash chromatography (120 g column, loaded in DCM, eluted with DCM) gave ((4-bromo-2-methoxybenzyl)oxy)(tert-butyl)dimethylsilane (6.766 g, 89% yield) as a colourless liquid.

¹H NMR (400 MHz, CHLOROFORM-d) δ 7.22 (d, J=8.2 Hz, 1H), 7.00 (dd, J=8.1, 1.8 Hz, 1H), 6.84 (d, J=1.8 Hz, 1H), 4.60-4.54 (m, 2H), 3.76-3.68 (m, 3H), 0.84 (s, 9H), 0.00 (s, 6H).

Step 2. A solution of ((4-bromo-2-methoxybenzyl)oxy)(tert-butyl)dimethylsilane (2.66 g, 8.03 mmol) in THF (40 mL) was cooled to −78° C. n-Butyllithium (3.37 mL, 8.43 mmol) was added portion-wise over 10 min. The resulting solution stirred for 15 min at −78° C. A solution of oxetan-3-one (0.550 g, 7.63 mmol) in THF (10 mL) was added portion wise over 5 min. The reaction mixture allowed to warm up to RT and stirred overnight. The reaction mixture was poured into saturated NaHCO₃ solution (100 mL) and extracted with EtOAc (3×40 mL). The combined organic phases were washed with brine (3×40 mL), dried (MgSO₄), filtered and concentrated. Flash chromatography (40 g column, loaded in DCM, 0 to 60% EtOAc in hexanes) gave 3-(4-(((tert-butyldimethylsilyl)oxy)methyl)-3-methoxyphenyl)oxetan-3-ol (1.36 g, 52% yield) as an oil, which solidified on standing.

¹H NMR (400 MHz, CHLOROFORM-d) δ 7.40 (d, J=7.7 Hz, 1H), 7.07 (dd, J=7.8, 1.7 Hz, 1H), 6.92 (d, J=1.5 Hz, 1H), 4.84-4.77 (m, 4H), 4.68-4.58 (m, 2H), 3.74 (s, 3H), 1.93-1.87 (m, 1H), 0.84 (s, 9H), 0.00 (s, 6H).

Step 3. A 20 mL scintillation vial was charged with 3-(4-(((tert-butyldimethylsilyl)-oxy)methyl)-3-methoxyphenyl)oxetan-3-ol (488 mg, 1.504 mmol), triethylamine (0.419 mL, 3.01 mmol), DMAP (18.37 mg, 0.150 mmol) and DCM (5 mL). Acetic anhydride (0.156 mL, 1.654 mmol) was added. The reaction mixture was stirred at RT for 1 h. The reaction mixture was evaporated to dryness, then re-dissolved in MeCN (2×5 mL) and evaporated to dryness again. The residue was dissolved in MeCN (2 mL). TBAF (3.01 mL, 3.01 mmol) [1N in THF] was added, and the reaction stirred for 1 hour. The reaction mixture was evaporated to dryness, then dissolved in MeCN (5 mL) and evaporated to dryness twice. The crude material was purified using flash chromatography (40 g column, loaded in DCM, 0 to 80% EtOAc in hexanes), giving 3-(4-(hydroxymethyl)-3-methoxyphenyl)oxetan-3-yl acetate (158 mg, 42% yield) as a solid.

¹H NMR (400 MHz, CD₃Cl) δ 7.33 (d, J=7.5 Hz, 1H), 7.06-6.99 (m, 1H), 5.07 (d, J=8.1 Hz, 2H), 4.96 (d, J=7.9 Hz, 2H), 4.71 (s, 2H), 3.93 (s, 3H), 2.17 (s, 3H), 1.86 (br s, 1H), 1.63 (br s, 1H).

LC/MS [M+H]⁺ 253.08.

Step 4. 3-(4-(hydroxymethyl)-3-methoxyphenyl)oxetan-3-yl acetate (150 mg, 0.595 mmol) was dissolved in DCM (5 mL). SOCl₂ (0.130 mL, 1.784 mmol) was added. The reaction stirred for 1 h at RT. The reaction mixture was evaporated to dryness, then dissolved and evaporated from MeCN (5 mL) twice. To a stirred solution of methyl (3-bromo-7-(butylamino)-1H-pyrazolo[4,3-d]pyrimidin-5-yl)carbamate (190 mg, 0.554 mmol) in DMF (2 mL) was added Cs₂CO₃ (361 mg, 1.108 mmol) followed by a solution of 3-(4-(chloromethyl)-3-methoxyphenyl)oxetan-3-yl acetate (150 mg, 0.554 mmol) in DMF (2 mL). The reaction mixture was stirred at RT overnight. The reaction was quenched with saturated NaHCO₃ solution (10 mL) and extracted with EtOAc (3×5 mL). The combined organic phases were washed with brine (4×5 mL), dried (MgSO₄), filtered and concentrated. Flash chromatography (40 g SiO₂ column, loaded in DCM, 0 to 15% MeOH in DCM) gave 3-(4-((3-bromo-7-(butylamino)-5-((methoxycarbonyl)amino)-1H-pyrazolo[4,3-d]pyrimidin-1-yl)methyl)-3-methoxyphenyl)oxetan-3-yl acetate (220 mg, 34%, ca. 50% pure, contaminated mainly with the 2-regioisomer byproduct).

LC/MS [M−H]⁻ 575.1, 577.0.

Step 5. 3-(4-((3-bromo-7-(butylamino)-5-((methoxycarbonyl)amino)-1H-pyrazolo[4,3-d]pyrimidin-1-yl)methyl)-3-methoxyphenyl)oxetan-3-yl acetate (65 mg, 0.113 mmol) was dissolved in EtOH (4 mL). Pd/C (50 mg) was added. The reaction mixture was evacuated and purged with hydrogen six times, then stirred overnight under a hydrogen atmosphere. The reaction mixture was filtered and the filtrate was evaporated to dryness. The residue was dissolved in dioxane (2 mL). NaOH (0.349 mL, 1.745 mmol) solution was added, and the reaction mixture was stirred at 80° C. for 3 hours. After cooling, the reaction mixture was neutralized with HCl and evaporated to dryness. The residue was dissolved in DMF (2 mL), filtered and purified via preparative LC/MS with the following conditions: Column: XBridge C18, 200 mm×19 mm, 5-μm particles; Mobile Phase A: 5:95 acetonitrile: water with 10-mM NH₄OAc; Mobile Phase B: 95:5 acetonitrile: water with 10-mM NH₄OAc; Gradient: a 0-minute hold at 8% B, 8-48% B over 20 min, then a 0-minute hold at 100% B; Flow Rate: 20 mL/min; Column Temperature: 25 C. Fraction collection was triggered by MS signals. Fractions containing the desired product were combined and dried via centrifugal evaporation, giving Compound 114 (2.1 mg, 4.7%).

### Example 12—Compound 115

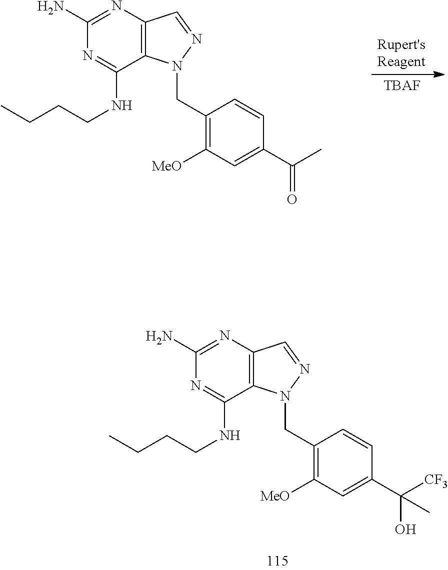

1-(4-((5-amino-7-(butylamino)-1H-pyrazolo[4,3-d]pyrimidin-1-yl)methyl)-3-methoxyphenyl)ethan-1-one (64 mg, 0.174 mmol) was dissolved in THF (5 mL). TBAF (0.521 mL, 0.521 mmol) was added and the reaction cooled in an ice bath. A solution of (trifluoromethyl)-trimethylsilane (Ruppert's reagent, 0.126 mL, 1.737 mmol) in THF (1 mL) was added portion wise. The reaction mixture allowed to warm slowly to RT and stirred for 1 h. More (trifluoro-methyl)trimethylsilane (0.126 mL, 1.737 mmol) was added. The reaction mixture was stirred for another 30 min. The reaction was quenched with water (0.5 mL) and MeOH (0.5 mL) and evaporated to dryness. The residue was dissolved in DMF (2 mL), filtered and purified via preparative LC/MS with the following conditions: Column: XBridge C18, 200 mm×19 mm, 5-μm particles; Mobile Phase A: 5:95 acetonitrile: water with 0.1% TFA; Mobile Phase B: 95:5 acetonitrile: water with 0.1% TFA; Gradient: a 0-minute hold at 12% B, 12-52% B over 25 min, then a 0-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge Phenyl, 200 mm×19 mm, 5-μm particles; Mobile Phase A: 5:95 acetonitrile: water with 10-mM NH₄OAc; Mobile Phase B: 95:5 acetonitrile: water with 10-mM NH₄OAc; Gradient: a 0-minute hold at 28% B, 28-68% B over 20 min, then a 0-minute hold at 100% B; Flow Rate: 20 mL/min; Column Temperature: 25° C. Fraction collection was triggered by MS signals. Fractions containing the desired product were combined and dried via centrifugal evaporation, giving Compound 115 (5.0 mg, 21% yield).

### Example 13—Compound 116

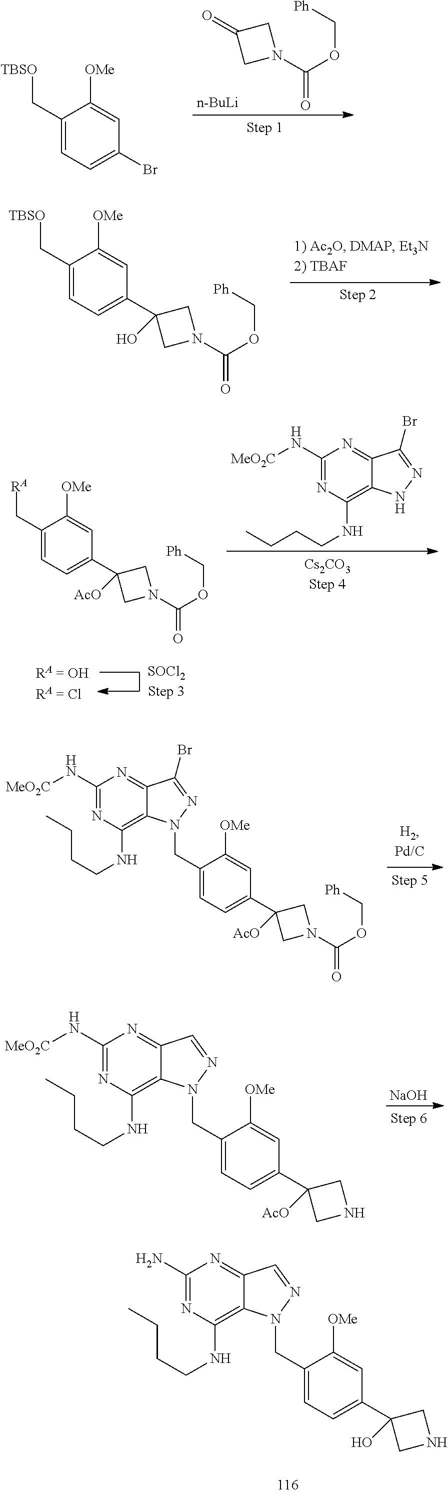

Step 1. A solution of ((4-bromo-2-methoxybenzyl)oxy)(tert-butyl)dimethylsilane (3 g, 9.05 mmol) in THF (40 mL) was cooled to −78° C. n-Butyllithium (3.80 mL, 9.51 mmol) was added portion wise over 10 min. The resulting solution was stirred for 15 min at −78° C. A solution of benzyl 3-oxoazetidine-1-carboxylate (1.765 g, 8.60 mmol) in THF (10 mL) was added portion wise over 5 min. The reaction mixture was allowed to warm to RT and stirred overnight. The reaction mixture was poured into saturated NaHCO₃ solution (100 mL) and extracted with EtOAc (3×40 mL). The combined organic phases were washed with brine (3×40 mL), dried (MgSO₄), filtered and concentrated. Flash chromatography (40 g SiO₂ column, loaded in DCM, 0 to 60% EtOAc in hexanes) gave benzyl 3-(4-(((tert-butyldimethylsilyl)oxy)methyl)-3-methoxy-phenyl)-3-hydroxyazetidine-1-carboxylate (679 mg, 16.39% yield) as an oil.

¹H NMR (400 MHz, CHLOROFORM-d) δ 7.38 (d, J=7.9 Hz, 1H), 7.27-7.17 (m, 5H), 6.95 (dd, J=7.9, 1.5 Hz, 1H), 6.81 (d, J=1.5 Hz, 1H), 5.03 (s, 2H), 4.63 (s, 2H), 4.28-4.11 (m, 4H), 3.71 (s, 3H), 0.84 (s, 9H), 0.00 (s, 6H).

Step 2. A 20 mL scintillation vial was charged with benzyl 3-(4-(((tert-butyldimethyl-silyl)oxy)methyl)-3-methoxyphenyl)-3-hydroxyazetidine-1-carboxylate (680 mg, 1.486 mmol), triethylamine (0.414 mL, 2.97 mmol), DMAP (18.15 mg, 0.149 mmol) and DCM (5 mL). Acetic anhydride (0.154 mL, 1.634 mmol) was added, and the reaction was stirred at RTfor 1 h. The reaction mixture was evaporated to dryness, dissolved in MeCN (5 mL) and evaporated to dryness twice. The residue was dissolved in MeCN (4 mL). TBAF (2.97 mL, 2.97 mmol, 1N in THF) was added, and the reaction mixture was stirred for 1 h at RT. The reaction mixture was evaporated to dryness then dissolved in MeCN (5 mL) and evaporated to dryness twice. The crude material was purified using flash chromatography (40 g SiO₂ column, loaded in DCM, 0 to 60% EtOAc in hexanes), giving benzyl 3-acetoxy-3-(4-(hydroxymethyl)-3-methoxyphenyl)-azetidine-1-carboxylate (260 mg, 45% yield) as an oil.

¹H NMR (400 MHz, CHLOROFORM-d) δ 7.38-7.27 (m, 6H), 6.95 (dd, J=7.7, 1.8 Hz, 1H), 6.88 (d, J=1.5 Hz, 1H), 5.13 (s, 2H), 4.66 (s, 2H), 4.47-4.39 (m, 4H), 3.86 (s, 3H), 2.10 (s, 3H).

Step 3. Benzyl 3-acetoxy-3-(4-(hydroxymethyl)-3-methoxyphenyl)azetidine-1-carboxylate (260 mg, 0.675 mmol) was dissolved in DCM (5 mL). SOCl₂ (0.059 mL, 0.810 mmol) was added and the reaction was stirred at RT for 1 h. The reaction mixture was evaporated to dryness, dissolved in MeCN (5 mL) and evaporated again, giving benzyl 3-acetoxy-3-(4-(chloromethyl)-3-methoxyphenyl)azetidine-1-carboxylate (270 mg, 0.669 mmol, 99% yield) as a colorless oil.

Step 4. A 20 mL scintillation vial was charged with methyl (3-bromo-7-(butylamino)-1H-pyrazolo[4,3-d]pyrimidin-5-yl)carbamate (200 mg, 0.583 mmol), Cs₂CO₃ (380 mg, 1.166 mmol) and DMF (2 mL) and cooled in an ice bath. A solution of benzyl 3-acetoxy-3-(4-(chloromethyl)-3-methoxyphenyl)azetidine-1-carboxylate (130 mg, 0.322 mmol) in DMF (3 mL) was added. The reaction mixture was allowed to warm slowly to RT and stirred for 24 h. Water (10 mL) was added, and the reaction mixture extracted into EtOAc (3×5 ml). The combined organic phases were washed with brine (4×5 ml), dried (MgSO₄), filtered and concentrated. Flash chromatography (24 g SiO₂ column, loaded in DCM, 0 to 10% MeOH in DCM) gave benzyl 3-acetoxy-3-(4-((3-bromo-7-(butylamino)-5-((methoxycarbonyl)amino)-1H-pyrazolo[4,3-d]pyrimidin-1-yl)methyl)-3-methoxyphenyl)azetidine-1-carboxylate (163 mg, 20% yield, ca. 50%, contaminated with the 2-regioisomer).

LC/MS [M−H]⁻ 708.0, 710.0.

Step 5. To a solution of benzyl 3-acetoxy-3-(4-((3-bromo-7-(butylamino)-5-((methoxycarbonyl)amino)-1H-pyrazolo[4,3-d]pyrimidin-1-yl)methyl)-3-methoxyphenyl)-azetidine-1-carboxylate (163 mg, 0.229 mmol, mixture of 1- and 2-regioisomers) in ethanol (20 mL) was added 10% Pd/C (100 mg). The reaction mixture was evacuated and purged with hydrogen six times, then stirred for 2 days under a hydrogen atmosphere. The reaction mixture was filtered and evaporated to dryness. Flash chromatography (40 g SiO₂ column, loaded in DCM, 0 to 20% MeOH in DCM) gave 3-(4-((7-(butylamino)-5-((methoxycarbonyl)amino)-1H-pyrazolo[4,3-d]pyrimidin-1-yl)methyl)-3-methoxyphenyl)azetidin-3-yl acetate (20 mg, 0.040 mmol, 17.52% yield) as a solid.

¹H NMR (400 MHz, DMSO-d₆) δ 9.63 (br s, 1H), 7.87 (s, 1H), 7.17-6.92 (m, 3H), 6.57 (d, J=7.9 Hz, 1H), 5.72 (s, 2H), 4.28-4.16 (m, 4H), 3.86 (s, 3H), 3.63 (s, 3H), 3.55-3.47 (m, 2H), 2.08 (s, 3H), 1.56 (quin, J=7.3 Hz, 2H), 1.25 (sxt, J=7.4 Hz, 2H), 0.91-0.84 (m, 3H).

LC/MS [M+H]⁺ 498.25.

Step 6. 3-(4-((7-(butylamino)-5-((methoxycarbonyl)amino)-1H-pyrazolo[4,3-d]pyrimidin-1-yl)methyl)-3-methoxyphenyl)azetidin-3-yl acetate (20 mg, 0.040 mmol) was dissolved in dioxane (2 mL). NaOH (0.201 mL, 1.005 mmol) was added, and the reaction mixture was stirred at 80° C. for 2 h. After cooling, the reaction mixture was neutralized with HCl and evaporated to dryness. The residue was dissolved in DMF (2 mL), filtered and purified via preparative LC/MS with the following conditions: Column: XBridge C18, 200 mm×19 mm, 5-μm particles; Mobile Phase A: 5:95 acetonitrile: water with NH₄OAc; Mobile Phase B: 95:5 acetonitrile: water with NH₄OAc; Gradient: a 0-minute hold at 4% B, 4-44% B over 20 min, then a 0-minute hold at 100% B; Flow Rate: 20 mL/min; Column Temperature: 25 C. Fraction collection was triggered by MS signals. Fractions containing the desired product were combined and dried via centrifugal evaporation, giving Compound 116 (3.5 mg, 22% yield).

### Example 14—Compound 117

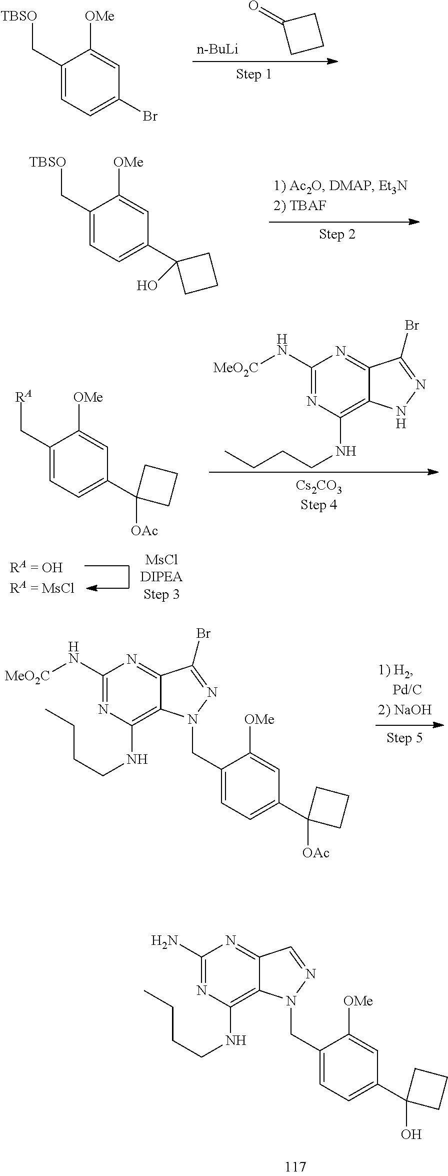

Step 1. A solution of ((4-bromo-2-methoxybenzyl)oxy)(tert-butyl)dimethylsilane (2.75 g, 8.30 mmol) in THF (40 mL) was cooled to −78° C. n-Butyllithium (3.49 mL, 8.72 mmol) was added portion wise over 10 min. The resulting solution was stirred for 15 in at −78° C. A solution of cyclobutanone (0.611 g, 8.72 mmol) in THF (10 mL) was added portion wise over 5 in. The reaction mixture was allowed to warm to RT, stirred overnight, and poured into saturated NaHCO₃ solution (100 ml). Extraction with EtOAc (3×40 mL) afforded combined organic phases that were washed with brine (3×40 ml), dried (MgSO₄), filtered and concentrated. Flash chromatography (80 g SiO₂ column, loaded in DCM, 0 to 25% EtOAc in hexanes) gave 1-(4-(((tert-butyldimethylsilyl)oxy)methyl)-3-methoxyphenyl)cyclobutan-1-ol (2.015 g, 75% yield) as an oil, which solidified on standing.

¹H NMR (400 MHz, CHLOROFORM-d) δ 7.35 (d, J=7.9 Hz, 1H), 6.98 (dd, J=7.7, 1.5 Hz, 1H), 6.87 (d, J=1.5 Hz, 1H), 4.64 (s, 2H), 3.73 (s, 3H), 2.50-2.42 (m, 2H), 2.30-2.21 (m, 2H), 1.95-1.81 (m, 1H), 1.64-1.52 (m, 1H), 0.86-0.83 (m, 9H), 0.00 (s, 6H).

Step 2. A 20 mL scintillation vial was charged with 1-(4-(((tert-butyldimethylsilyl)-oxy)methyl)-3-methoxyphenyl)cyclobutan-1-ol (2 g, 6.20 mmol), triethylamine (1.729 mL, 12.40 mmol), DMAP (0.076 g, 0.620 mmol) and DCM (20 mL). Acetic anhydride (0.644 mL, 6.82 mmol) was added. The reaction mixture was stirred at RT for 2 h, and evaporated to dryness. The residue was dissolved in MeCN (5 mL) and evaporated to dryness twice. The residue was re-dissolved in MeCN (8 mL). TBAF (12.40 mL, 12.40 mmol, 1N in THF was added, and the reaction mixture was stirred for 1 h. The reaction mixture was evaporated to dryness. The residue was dissolved in MeCN (5 mL) and evaporated to dryness twice. The crude material was purified using flash chromatography, giving 1-(4-(hydroxymethyl)-3-methoxyphenyl-cyclobutyl acetate (1.0 g, 64% yield) as an oil.

¹H NMR (400 MHz, CHLOROFORM-d) δ 7.26 (d, J=1.8 Hz, 1H), 7.07 (dd, J=7.8, 1.7 Hz, 1H), 6.98 (d, J=1.3 Hz, 1H), 4.67 (s, 2H), 3.89 (s, 3H), 2.70-2.55 (m, 4H), 2.09-1.92 (m, 4H), 1.74 (dquin, J=11.2, 8.8 Hz, 1H).

Step 3. 1-(4-(Hydroxymethyl)-3-methoxyphenyl)cyclobutyl acetate (500 mg, 1.998 mmol) was dissolved in DCM (10 mL) and cooled in an ice bath. DIPEA (0.436 mL, 2.497 mmol) was added, followed by methanesulfonyl chloride (0.467 mL, 5.99 mmol). The reaction mixture was stirred at 0° C. for 30 min, then at RT overnight. The reaction mixture was quenched with saturated NaHCO₃ solution (10 mL) and extracted with DCM (2×5 ml). The combined organic phases were washed with NaHCO₃ solution (10 mL) and brine (2×10 ml), dried (MgSO₄), filtered and concentrated, giving 1-(3-methoxy-4-(((methylsulfonyl)oxy)methyl)-phenyl)cyclobutyl acetate (498 mg, 76% yield) as an oil.

Step 4. To a stirred solution of methyl (3-bromo-7-(butylamino)-1H-pyrazolo[4,3-d]pyrimidin-5-yl)carbamate (650 mg, 1.894 mmol) in DMF (2 mL) at 0° C. was added Cs₂CO₃ (1234 mg, 3.79 mmol), followed by a solution of 1-(3-methoxy-4-(((methylsulfonyl)oxy)-methyl)phenyl)cyclobutyl acetate (498 mg, 1.515 mmol) in DMF (1 ml). The reaction mixture was allowed to warm to RT, stirred for 72 h, and poured into saturated NaHCO₃ solution (50 ml). Extraction with EtOAc (3×40 mL) afforded organic phases that were combined and washed with brine (4×30 ml), dried (MgSO₄), filtered and concentrated. Flash chromatography (40 g SiO₂ column, loaded in DCM, 0 to 65% EtOAc in hexanes) gave 1-(4-((3-bromo-7-(butylamino)-5-((methoxycarbonyl)amino)-1H-pyrazolo[4,3-d]pyrimidin-1-yl)methyl)-3-methoxyphenyl)cyclobutyl acetate (242 mg, 22% yield) as a solid.

¹H NMR (400 MHz, DMSO-d₆) δ 9.86 (s, 1H), 7.30 (t, J=5.6 Hz, 1H), 7.02-6.96 (m, 2H), 6.73 (d, J=7.7 Hz, 1H), 5.68 (s, 2H), 3.80 (s, 3H), 3.63 (s, 3H), 3.60-3.50 (m, 2H), 3.32 (s, 2H), 2.59-2.44 (m, 2H), 1.96 (s, 3H), 1.94-1.86 (m, 1H), 1.77-1.65 (m, 1H), 1.59 (quin, J=7.3 Hz, 2H), 1.27 (sxt, J=7.4 Hz, 2H), 0.88 (t, J=7.4 Hz, 3H). LC/MS [M+H]⁺ 575.37, 577.37.

Step 5. 1-(4-((3-bromo-7-(butylamino)-5-((methoxycarbonyl)amino)-1H-pyrazolo[4,3-d]pyrimidin-1-yl)methyl)-3-methoxyphenyl)cyclobutyl acetate (200 mg, 0.348 mmol) was dissolved in EtOH (25 mL). 10% Pd/C (50 mg) was added. The reaction mixture was evacuated and purged six times with hydrogen, stirred overnight under a hydrogen atmosphere, filtered, and evaporated to dryness. The residue was dissolved in dioxane (2 mL). NaOH (0.242 mL, 1.208 mmol) was added, and the reaction was heated to 80° C. and maintained at this temperature for 4 hours. After cooling, the reaction mixture was neutralized with 5N HCl, then evaporated to dryness. The residue was dissolved in DMF (2 mL), filtered and purified via preparative LC/MS with the following conditions: Column: XBridge C18, 200 mm×19 mm, 5-μm particles; Mobile Phase A: 5:95 acetonitrile: water with NH₄OAc; Mobile Phase B: 95:5 acetonitrile: water with NH₄OAc; Gradient: a 0-minute hold at 16% B, 16-56% B over 20 min, then a 0-minute hold at 100% B; Flow Rate: 20 mL/min; Column Temperature: 25 C. Fraction collection was triggered by MS and UV signals. Fractions containing the desired product were combined and dried via centrifugal evaporation, giving Compound 117 (22.5 mg, 16% yield).

### Example 15—Compound 118

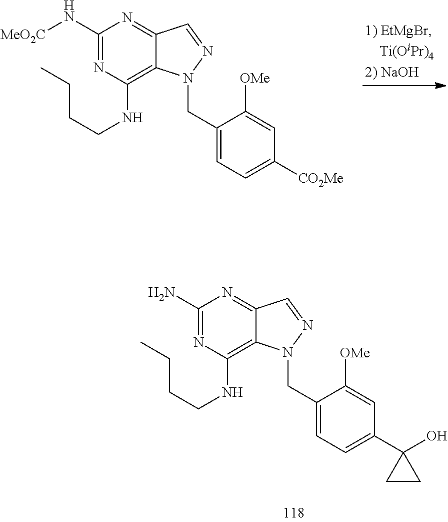 [SMILES: CCCCNc1nc(NC(=O)OC)nc2cnn(Cc3ccc(C(=O)OC)cc3OC)c12 ; CCCCNc1nc(N)nc2cnn(Cc3ccc(C4(O)CC4)cc3OC)c12]

To a solution of titanium(IV) isopropoxide (257 mg, 0.904 mmol) in THF (8 mL) at −78° C. was added ethylmagnesium bromide (2.71 mL, 2.71 mmol), portion wise over 10 min. The reaction was stirred at −78° C. for 60 min. Then a solution of methyl 4-((7-(butylamino)-5-((methoxycarbonyl)amino)-1H-pyrazolo[4,3-d]pyrimidin-1-yl)methyl)-3-methoxybenzoate (100 mg, 0.226 mmol) in THF (1 mL) was added. The reaction was allowed to warm slowly to RT and stirred overnight. The reaction was quenched with water (20 mL), filtered and extracted with EtOAc (3×5 mL). The combined organic phases were washed with brine (3×5 mL), dried (MgSO₄), filtered and concentrated. The residue was dissolved in dioxane (2 mL). NaOH (0.436 mL, 2.179 mmol) was added, and the reaction mixture was stirred at 80° C. for 2 hours. After cooling it was neutralized with HCl and evaporated to dryness. The crude material was dissolved in DMF (2 mL), filtered and purified via preparative LC/MS with the following conditions: Column: XBridge C18, 200 mm×19 mm, 5-μm particles; Mobile Phase A: 5:95 acetonitrile: water with NH₄OAc; Mobile Phase B: 95:5 acetonitrile: water with NH₄OAc; Gradient: a 0-minute hold at 19% B, 19-59% B over 20 min, then a 0-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×19 mm, 5-μm particles; Mobile Phase A: 5:95 acetonitrile: water with NH₄OAc; Mobile Phase B: 95:5 acetonitrile: water with NH₄OAc; Gradient: a 0-minute hold at 20% B, 20-60% B over 20 min, then a 0-minute hold at 100% B; Flow Rate: 20 mL/min; Column Temperature: 25 C. Fraction collection was triggered by MS and UV signals. Fractions containing the desired product were combined and dried via centrifugal evaporation, giving Compound 118 (9.4 mg, 11% yield).

### Example 16—Compound 109

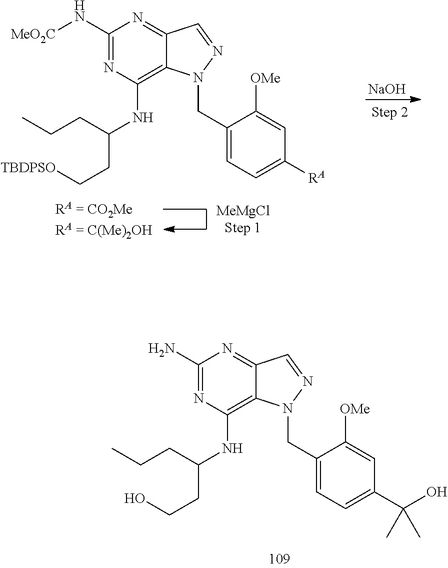

Step 1. A solution of methyl (S)-4-((7-((1-((tert-butyldiphenylsilyl)oxy)hexan-3-yl)amino)-5-((methoxycarbonyl)amino)-1H-pyrazolo[4,3-d]pyrimidin-1-yl)methyl)-3-methoxybenzoate (30 mg, 0.041 mmol; US 2020/0038403, FIG. 3A, compound 24) in THF (1 mL) was treated with methylmagnesium chloride in THF (0.069 mL, 0.207 mmol). The reaction mixture was stirred for 1 h, after which LCMS showed completion of the reaction. The reaction was quenched with MeOH (1 mL) and the solvent was evaporated. The crude product was taken to next step as-is.

Step 2. A solution of methyl (S)-(7-((1-((tert-butyldiphenylsilyl)oxy)hexan-3-yl)amino)-1-(4-(2-hydroxypropan-2-yl)-2-methoxybenzyl)-1H-pyrazolo[4,3-d]pyrimidin-5-yl)carbamate (26 mg, 0.036 mmol) in dioxane (0.5 mL) was treated with NaOH (0.179 mL, 0.179 mmol) and heated at 80° C. overnight, after which at which LCMS showed de-protection of carbamate and TBDPS. The reaction was neutralized to pH 7 by the slow addition of 6M HCl and the solvent was evaporated. The crude material was purified via preparative LC/MS with the following conditions: Column: XBridge C18, 200 mm×19 mm, 5-μm particles; Mobile Phase A: 5:95 acetonitrile: water with 10-mM NH₄OAc; Mobile Phase B: 95:5 acetonitrile: water with 10-mM NH₄OAc; Gradient: a 0-minute hold at 11% B, 11-51% B over 20 min, then a 4-minute hold at 100% B; Flow Rate: 20 mL/min; Column Temperature: 25 C. Fraction collection was triggered by MS and UV signals. Fractions containing the desired Compound 109 were combined and dried via centrifugal evaporation.

### Example 17—Compound 119

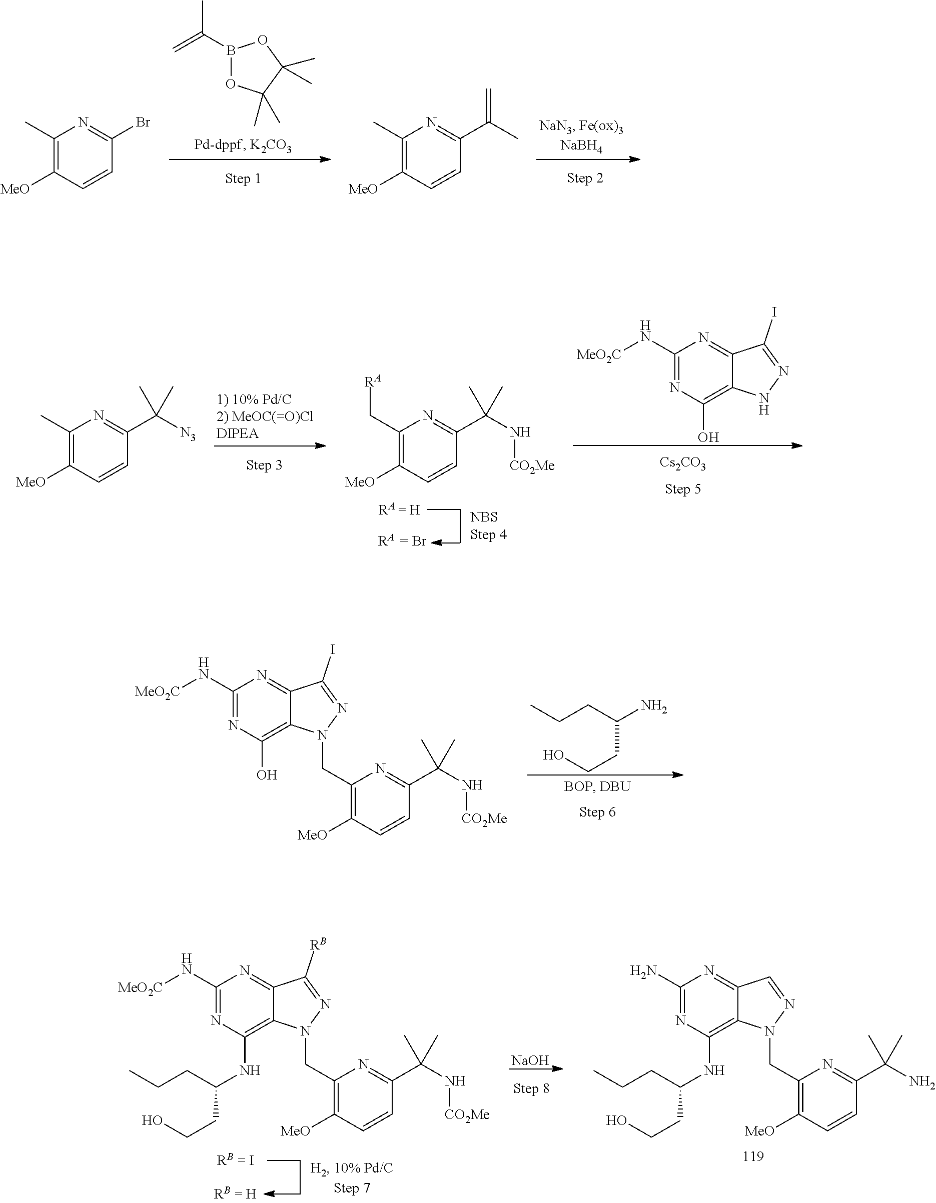

Step 1. A vial was charged with 4,4,5,5-tetramethyl-2-(prop-1-en-2-yl)-1,3,2-dioxaborolane (0.832 g, 4.95 mmol), 6-bromo-3-methoxy-2-methylpyridine (1 g, 4.95 mmol), [1,1′-bis(diphenylphosphino)ferrocene]dichloropalladium(II) (0.362 g, 0.495 mmol), dioxane (9.90 ml) and water (2.475 ml) The reaction mixture was heated at 65° C. overnight. The reaction mixture was poured into saturated NaHCO₃ solution (10 mL) and extracted with EtOAc (3×5 mL). The combined organic phases were washed with brine (3×5 mL), dried (MgSO₄), filtered and concentrated. The crude material was purified using flash chromatography (40 g SiO₂ column, loaded in DCM, 0 to 20% EtOAc in hexanes), giving 3-methoxy-2-methyl-6-(prop-1-en-2-yl)pyridine (366 mg, 45% yield) as an oil.

LC/MS [M+H]⁺ 164.1.

Step 2. A suspension of Iron(III) oxalate hexahydrate (1766 mg, 4.48 mmol in water (70 mL) was stirred for 4 hours to dissolve the solid material. The solution was then cooled in an ice bath and degassed for 10 min with nitrogen. A solution of sodium azide (437 mg, 6.73 mmol) in EtOH (35 mL) was added, followed by a solution of 3-methoxy-2-methyl-6-(prop-1-en-2-yl)pyridine (366 mg, 2.242 mmol) in EtOH (35 mL). The reaction mixture was stirred at 0° C. for 5 min; then sodium borohydride (254 mg, 6.73 mmol) was added in two portions 5 min apart. The reaction was stirred for 30 min and quenched with ammonia solution (40 mL) and stirred for a further 30 min at RT. The product was extracted with DCM (3×50 mL) and the combined organic phases were washed with brine (50 mL), dried (MgSO₄), filtered and concentrated. The crude material was purified using flash chromatography, giving 6-(2-azidopropan-2-yl)-3-methoxy-2-methylpyridine (290 mg, 63% yield) as colorless liquid.

LC/MS [M+H]⁺ 207.2.

Step 3. To a solution of 6-(2-azidopropan-2-yl)-3-methoxy-2-methylpyridine (290 mg, 1.406 mmol) in ethanol (7 mL) was added 10% palladium on carbon (74.8 mg, 0.070 mmol). The reaction mixture was stirred under hydrogen atmosphere for 4 h, filtered through CELITE™ and concentrated. The residue was dissolved in DCM (7 mL) and cooled to 0° C. DIPEA (0.737 mL, 4.22 mmol) was added, followed by methyl chloroformate (0.218 mL, 2.81 mmol). The reaction mixture was stirred at RT overnight, quenched with saturated NaHCO₃ solution (20 mL) and extracted with DCM (3×10 mL). The combined organic phases were washed with brine (10 mL), dried (MgSO₄) and concentrated. The crude material was purified using flash chromatography, providing methyl (2-(5-methoxy-6-methylpyridin-2-yl)propan-2-yl)carbamate (219 mg, 65% yield) as an oil.

LC/MS [M+H]⁺ 239.2.

Step 4. To a solution of NBS (164 mg, 0.919 mmol) and AIBN (15.09 mg, 0.092 mmol) in carbon tetrachloride (4 mL) was added methyl (2-(5-methoxy-6-methylpyridin-2-yl)propan-2-yl)carbamate (219 mg, 0.919 mmol). The reaction mixture was stirred at 75° C. for 3 h. After cooling, it was evaporated to dryness and purified using flash chromatography (24 g SiO₂ column, loaded in DCM, 0 to 50% EtOAc in hexanes), giving methyl (2-(6-(bromomethyl)-5-methoxypyridin-2-yl)propan-2-yl)carbamate (273 mg, 94% yield).

LC/MS [M+H]⁺ 317.1, 319.1.

Step 5. To a stirred solution of methyl (7-hydroxy-3-iodo-1H-pyrazolo[4,3-d]pyrimidin-5-yl)carbamate (288 mg, 0.861 mmol) in DMF (5738 μl) was added Cs₂CO₃ (308 mg, 0.947 mmol) followed by methyl (7-hydroxy-3-iodo-1H-pyrazolo[4,3-d]pyrimidin-5-yl)carbamate (288 mg, 0.861 mmol). The reaction mixture was stirred at RT overnight, diluted with EtOAc (50 ml), washed with brine (2×20 ml), dried (MgSO₄), concentrated, and purified using flash chromatography (0-20% MeOH/DCM). Product-containing fractions were concentrated to afford methyl (7-hydroxy-3-iodo-1-((3-methoxy-6-(2-((methoxycarbonyl)amino)propan-2-yl)pyridin-2-yl)methyl)-1H-pyrazolo[4,3-d]pyrimidin-5-yl)carbamate (374 mg, 76% yield) as a solid.

LC/MS [M+H]⁺ 572.2.

Step 6. To a stirred solution of methyl (7-hydroxy-3-iodo-1-((3-methoxy-6-(2-((methoxycarbonyl)amino)propan-2-yl)pyridin-2-yl)methyl)-1H-pyrazolo[4,3-d]pyrimidin-5-yl)carbamate (94 mg, 0.165 mmol), (S)-3-aminohexan-1-ol, HCl (50.6 mg, 0.329 mmol) and BOP (109 mg, 0.247 mmol) in DMSO (1645 l) was added DBU (99 pI, 0.658 mmol). The reaction mixture was stirred at RT overnight, diluted with EtOAc (30 mL), washed with brine (4×20 mL), dried (MgSO₄) and concentrated. The crude material was purified using flash chromatography (24 g SiO₂ column, loaded in DCM, 0 to 10% MeOH/DCM), giving methyl (S)-(7-((1-hydroxy-hexan-3-yl)amino)-3-iodo-1-((3-methoxy-6-(2-((methoxycarbonyl)amino)propan-2-yl)pyridin-2-yl)methyl)-1H-pyrazolo[4,3-d]pyrimidin-5-yl)carbamate (64 mg, 58.0% yield) as solid.

¹H NMR (400 MHz, DMSO-d₆) δ 9.84 (s, 1H), 7.41 (d, J=8.8 Hz, 1H), 7.18 (d, J=8.6 Hz, 1H), 7.12 (s, 1H), 6.66 (d, J=8.6 Hz, 1H), 5.98 (d, J=17.6 Hz, 1H), 5.80 (d, J=17.6 Hz, 1H), 4.43 (br d, J=6.8 Hz, 1H), 4.34 (dd, J=6.6, 4.6 Hz, 1H), 3.87 (s, 3H), 3.62 (s, 3H), 3.46-3.40 (m, 4H), 1.70-1.59 (m, 2H), 1.55-1.45 (m, 1H), 1.41 (br d, J=9.2 Hz, 1H), 1.14-0.96 (m, 8H), 0.73-0.68 (m, 3H).

LC/MS [M+H]⁺ 671.3.

Step 7. Methyl (S)-(7-((1-hydroxyhexan-3-yl)amino)-3-iodo-1-((3-methoxy-6-(2-((methoxycarbonyl)amino)propan-2-yl)pyridin-2-yl)methyl)-1H-pyrazolo[4,3-d]pyrimidin-5-yl)carbamate (64 mg, 0.095 mmol) was dissolved in EtOH (4772 μl). 10% palladium on carbon (7.11 mg, 6.68 μmol) was added. The reaction mixture was evacuated, purged three times with hydrogen, and stirred under a hydrogen atmosphere overnight. The reaction mixture was filtered through CELITE™, washing with ethanol (10 mL). The filtrate was evaporated to give methyl (S)-(7-((1-hydroxyhexan-3-yl)amino)-1-((3-methoxy-6-(2-((methoxycarbonyl)amino)-propan-2-yl)pyridin-2-yl)methyl)-1H-pyrazolo[4,3-d]pyrimidin-5-yl)carbamate (52.0 mg, 100% yield) as a solid.

LC/MS [M+H]⁺=545.4.

Step 8. To a stirred solution of methyl (S)-(7-((1-hydroxyhexan-3-yl)amino)-1-((3-methoxy-6-(2-((methoxycarbonyl)amino)propan-2-yl)pyridin-2-yl)methyl)-1H-pyrazolo[4,3-d]pyrimidin-5-yl)carbamate (52 mg, 0.095 mmol) in MeOH (955 μl) was added NaOH (191 μL, 1.910 mmol). The reaction mixture was stirred at 80° C. overnight, concentrated and re-dissolved in dioxane (1 mL). It was then treated with NaOH (0.2 mL) and stirred at 100° C. overnight. The reaction was cooled to 0° C., quenched with HCl (159 μL, 1.910 mmol) and concentrated. The crude material was dissolved in DMF (2 ml), filtered and purified via preparative LC/MS with the following conditions: Column: XBridge C18, 200 mm×19 mm, 5-μm particles; Mobile Phase A: 5:95 acetonitrile: water with NH₄OAc; Mobile Phase B: 95:5 acetonitrile: water with NH₄OAc; Gradient: a 0-minute hold at 0% B, 0-40% B over 25 min, then a 0-minute hold at 100% B; Flow Rate: 20 ml/min; Column Temperature: 25 C. Fraction collection was triggered by MS signals. Fractions containing the desired product were combined and dried via centrifugal evaporation, giving Compound 119 (6.2 mg, 15% yield).

### Example 18—Compound 120

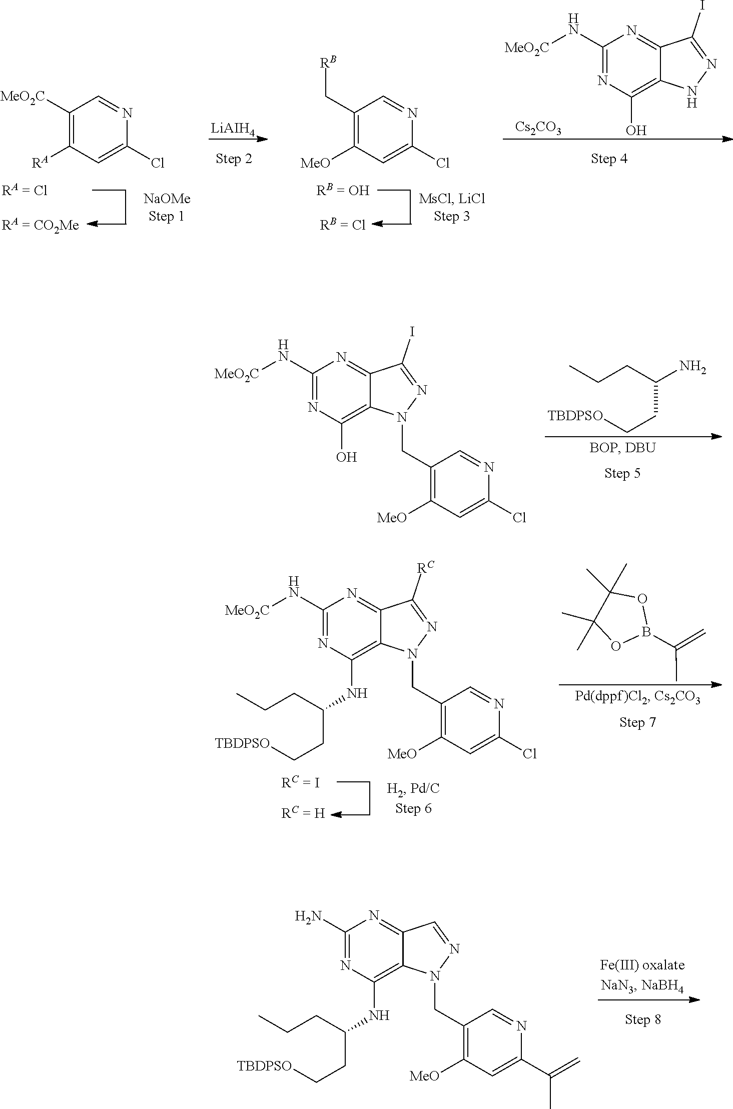

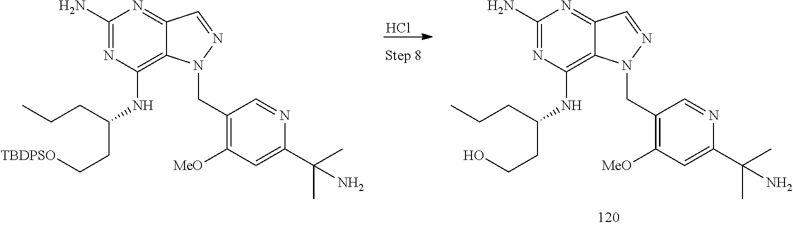

Step 1. To a stirred solution of methyl 4,6-dichloronicotinate (5 g, 24.27 mmol) in THF (50 ml), sodium methanolate (5.41 mL, 29.1 mmol) was added dropwise over 2 min at 0° C. The reaction mixture was stirred at 0° C. for 5 min and then at RT for 12 h. The reaction mixture was partitioned between water (50 mL) and ethyl acetate (50 mL). The organic layer was separated out and the aqueous layer was extracted with EtOAc (2×30 mL). The combined organic layers were washed with brine solution (100 mL), dried over anhydrous Na₂SO₄, filtered and concentrated under vacuum to give the crude product, which was purified by flash chromatography (40 g silica gel column, 30% EtOAc in petroleum ether) to afford methyl 6-chloro-4-methoxynicotinate (3.4 g, 16.86 mmol, 69.5% yield) as an off-white solid.

¹H NMR (400 MHz, DMSO-d₆) δ=8.57 (s, 1H), 7.37 (s, 1H), 3.96 (s, 3H), 3.82 (s, 3H). LC-MS m/z 202.2 [M+H]⁺.

Step 2. To a stirred solution of methyl 6-chloro-4-methoxynicotinate (3.2 g, 15.87 mmol) in THF (40 mL) at 0° C., was added LiAlH₄ (31.7 mL, 31.7 mmol) in a dropwise fashion over 10 min. After the addition was over, the reaction mixture was allowed to warm to RT and stirred for 2 h. The reaction mixture was cooled and quenched by the successive dropwise addition of water (1.0 ml), 15% aqueous NaOH (1.0 mL) and water (2.0 ml). After being stirred for 30 min, the mixture was filtered through a pad of CELITE™, which was washed with excess EtOAc. The filtrate was concentrated under reduced pressure to give a residue, which was washed with cold ether (15 mL) and dried to afford (6-chloro-4-methoxypyridin-3-yl)methanol (2.2 g, 12.67 mmol, 80% yield) as an off-white solid.

¹H NMR (400 MHz, DMSO-d₆) δ=8.20-8.14 (m, 1H), 7.15-7.07 (m, 1H), 5.20 (t, J=5.5 Hz, 1H), 4.46 (d, J=5.5 Hz, 2H), 3.90-3.86 (m, 3H). LC-MS m/z 174.2 [M+H]⁺.

Step 3. To a stirred solution of (6-chloro-4-methoxypyridin-3-yl)methanol (2.9 g, 16.71 mmol) in DCM (30.0 ml), were added TEA (4.66 mL, 33.4 mmol), MsCl (2.60 mL, 33.4 mmol) and lithium chloride (anhydrous, 1.416 g, 33.4 mmol) at 0° C. The reaction mixture was stirred at 0° C. for 30 min and then at RT for 3 h. The reaction mixture was partitioned between DCM and water. The organic layer was washed with brine solution and dried over Na₂SO₄, filtered and concentrated under reduced pressure to afford 2-chloro-5-(chloromethyl)-4-methoxypyridine (3.0 g, 9.22 mmol, 55.2% yield) as a light brown oil.

¹H NMR (400 MHz, Chloroform-d) δ=8.24-8.22 (m, 1H), 6.86-6.84 (m, 1H), 4.59-4.54 (m, 2H), 3.96-3.95 (s, 3H). LC-MS m/z 192.0 [M+H]⁺.

Step 4. To a stirred solution of methyl (7-hydroxy-3-iodo-1H-pyrazolo[4,3-d]pyrimidin-5-yl)carbamate (5.0 g, 14.92 mmol) in DMF (50.0 mL) at 0° C., Cs₂CO₃ (9.72 g, 29.8 mmol) and 2-chloro-5-(chloromethyl)-4-methoxypyridine (2.87 g, 14.92 mmol) were added. The reaction mixture was stirred at 0° C. for 1 h. and then water was added. The precipitated solid was filtered and washed with excess of water followed by petroleum ether. The solid was dried under vacuum to afford methyl (1-((6-chloro-4-methoxypyridin-3-yl)methyl)-7-hydroxy-3-iodo-1H-pyrazolo[4,3-d]pyrimidin-5-yl)carbamate (4.9 g, 8.79 mmol, 58.9% yield) as an off-white solid.

¹H NMR (400 MHz, DMSO-d₆) δ=11.69 (br s, 1H), 11.40-11.34 (m, 1H), 7.97-7.94 (m, 1H), 7.23-7.19 (m, 1H), 5.70-5.67 (m, 2H), 3.88-3.84 (m, 3H), 3.78-3.74 (m, 3H). LC-MS m/z 490.8 [M+H]⁺.

Step 5. To a stirred solution of methyl (1-((6-chloro-4-methoxypyridin-3-yl)methyl)-7-hydroxy-3-iodo-1H-pyrazolo[4,3-d]pyrimidin-5-yl)carbamate (4.0 g, 8.15 mmol) in DMSO (30.0 mL), DBU (3.69 mL, 24.46 mmol), BOP (5.41 g, 12.23 mmol) and (S)-1-((tert-butyldiphenylsilyl)oxy)hexan-3-amine (2.90 g, 8.15 mmol) were added. The reaction mixture was stirred at 45° C. for 2 h and then partitioned between EtOAc and water. The organic layer was washed with brine solution, dried over Na₂SO₄, filtered and concentrated under reduced pressure to afford a residue. The crude compound was purified by ISCO Combiflash chromatography by eluting with 50-100% ethyl acetate in chloroform to afford methyl (S)-(7-((1-((tert-butyldiphenylsilyl)oxy)hexan-3-yl)amino)-1-((6-chloro-4-methoxypyridin-3-yl)methyl)-3-iodo-1H-pyrazolo[4,3-d]pyrimidin-5-yl)carbamate (2.89 g, 3.25 mmol, 39.8% yield) as a light brown solid.

¹H NMR (400 MHz, DMSO-d₆) δ=9.74 (s, 1H), 7.75 (s, 1H), 7.59-7.55 (m, 2H), 7.52-7.33 (m, 7H), 7.28-7.22 (m, 2H), 7.16 (s, 1H), 6.75 (d, J=8.5 Hz, 1H), 5.78-5.59 (m, 2H), 4.72-4.61 (m, 1H), 3.73 (s, 3H), 3.71-3.66 (m, 2H), 3.59 (s, 3H), 1.95-1.86 (m, 2H), 1.66-1.47 (m, 2H), 1.32-1.14 (m, 2H), 0.93 (s, 9H), 0.86-0.81 (m, 3H); LC-MS m/z 828.2 [M+H]⁺.

Step 6. To a stirred solution of methyl (S)-(7-((1-((tert-butyldiphenylsilyl)oxy)hexan-3-yl)amino)-1-((6-chloro-4-methoxypyridin-3-yl)methyl)-3-iodo-1H-pyrazolo[4,3-d]pyrimidin-5-yl)carbamate (1.65 g, 1.992 mmol) in a mixture of ethyl acetate (10.0 mL) and ethanol (10.0 mL), Pd/C (1.060 g, 0.996 mmol) was added. The reaction mixture was stirred at RT under hydrogen bladder pressure for 16 h. The reaction mixture was filtered through a CELITE” bed. The CELITE” bed was washed with excess of methanol. The filtrate was concentrated under reduced pressure to afford methyl (S)-(7-((1-((tert-butyldiphenylsilyl)oxy)hexan-3-yl)amino)-1-((6-chloro-4-methoxypyridin-3-yl)methyl)-1H-pyrazolo[4,3-d]pyrimidin-5-yl)carbamate (1.5 g, 1.730 mmol, 87% yield) as a light brown solid.

LC-MS m/z 702.2 [M+H]⁺.

Step 7. To a stirred solution of methyl (S)-(7-((1-((tert-butyldiphenylsilyl)oxy)hexan-3-yl)amino)-1-((6-chloro-4-methoxypyridin-3-yl)methyl)-1H-pyrazolo[4,3-d]pyrimidin-5-yl)carbamate (0.5 g, 0.712 mmol) in a mixture of 1,4-dioxane (4.0 mL) and water (1.0 mL), Cs₂CO₃ (0.696 g, 2.136 mmol), 4,4,5,5-tetramethyl-2-(prop-1-en-2-yl)-1,3,2-dioxaborolane (0.201 mL, 1.068 mmol) and PdCl₂(dppf).CH₂Cl₂ adduct (0.058 g, 0.071 mmol) were added. The reaction mixture was purged with nitrogen and stirred at 100° C. for 16 h. The reaction mixture was filtered through a CELITE” bed. The filtrate was partitioned between EtOAc and water. The organic layer was washed with brine solution, dried over Na₂SO₄, filtered and concentrated under reduced pressure to afford the residue. The crude compound was purified by ISCO Combiflash chromatography by eluting with 40-100% ethyl acetate in chloroform to afford (S)-N7-(1-((tert-Butyldiphenylsilyl)oxy)hexan-3-yl)-1-((4-methoxy-6-(prop-1-en-2-yl)pyridin-3-yl)methyl)-1H-pyrazolo[4,3-d]pyrimidine-5,7-diamine (162 mg, 0.229 mmol, 32.2% yield) as a brown semi-solid.

¹H NMR (400 MHz, DMSO-d₆) δ=7.61-7.48 (m, 7H), 7.43-7.34 (m, 4H), 7.31-7.25 (m, 2H), 7.17 (s, 1H), 5.86 (s, 1H), 5.77-5.49 (m, 4H), 5.27 (s, 1H), 4.56-4.45 (m, 1H), 3.86-3.83 (m, 3H), 3.69-3.61 (m, 2H), 2.08-2.05 (m, 3H), 1.83-1.76 (m, 2H), 1.49-1.40 (m, 2H), 1.16-1.05 (m, 2H), 0.96 (s, 9H), 0.80-0.74 (m, 3H).

LC-MS m/z 650.4 [M+H]⁺.

Step 8. Iron(III) oxalate hexahydrate (0.606 g, 1.539 mmol) was stirred in water (10.0 mL) at RT for 2 h to make homogeneous solution. This mixture was degassed with nitrogen for 10 min at 0° C. To this mixture THF (10.0 mL) and sodium azide (0.200 g, 3.08 mmol) were added followed by a solution of (S)-N7-(1-((tert-butyldiphenylsilyl)oxy)hexan-3-yl)-1-((4-methoxy-6-(prop-1-en-2-yl)pyridin-3-yl)methyl)-1H-pyrazolo[4,3-d]pyrimidine-5,7-diamine (0.2 g, 0.308 mmol) in THF (10.0 mL). The reaction mixture was stirred at 0° C. for 5 min. Subsequently, sodium borohydride (0.075 g, 1.969 mmol) was added in two lots over 10 min. The reaction mixture was stirred at 0° C. for 30 min, treated with ammonia solution and stirred at RT for 30 min. The reaction mixture was partitioned between DCM and water. The organic layer was washed with brine solution, dried over Na₂SO₄, filtered and concentrated under reduced pressure to afford a residue. The crude compound was purified by ISCO Combiflash chromatography by eluting with 0-20% methanol in chloroform to afford (S)-1-((6-(2-aminopropan-2-yl)-4-methoxypyridin-3-yl)methyl)-N7-(1-((tert-butyldiphenylsilyl)oxy)hexan-3-yl)-1H-pyrazolo[4,3-d]pyrimidine-5,7-diamine (130 mg, 0.177 mmol, 57.6% yield) as a light brown semi-solid.

LC-MS m/z 667.2 [M+H]⁺.

Step 9. To a stirred solution of (S)-1-((6-(2-aminopropan-2-yl)-4-methoxypyridin-3-yl)methyl)-N7-(1-((tert-butyldiphenylsilyl)oxy)hexan-3-yl)-1H-pyrazolo[4,3-d]pyrimidine-5,7-diamine (100.0 mg, 0.150 mmol) in MeOH (3.0 mL), conc. HCl (1.0 mL, 1.500 mmol) was added at 0° C. The reaction mixture was stirred at RT for 1 h and concentrated under reduced pressure to afford a residue. The residue was triturated with diethyl ether and petroleum ether, the solid was dried under vacuum. The crude compound was purified by reversed phase preparative LC/MS (Column: Waters XBridge C18, 19×150 mm, 5-μm particles; mobile phase A: 10 mM NH₄OAc; mobile phase B: acetonitrile; gradient: 15-55% B over 20 min, then a 5-minute hold at 100% B; flow: 15 mL/min.). The fraction collection was triggered by MS and UV signals. Fractions containing the desired product were combined and dried via centrifugal evaporation using a Genevac apparatus to afford Compound 120 (11.1 mg, 0.026 mmol, 17.28% yield).

### Example 19—Starting Materials and Intermediates

Chart 1 below show schemes for making compounds that could be useful as starting materials or intermediates for the preparation of TLR7 agonists disclosed herein. The schemes can be adapted to make other, analogous compounds that could be used as starting materials or intermediates. The reagents employed are well known in the art and in many instances their use has been demonstrated in the preceding Examples.

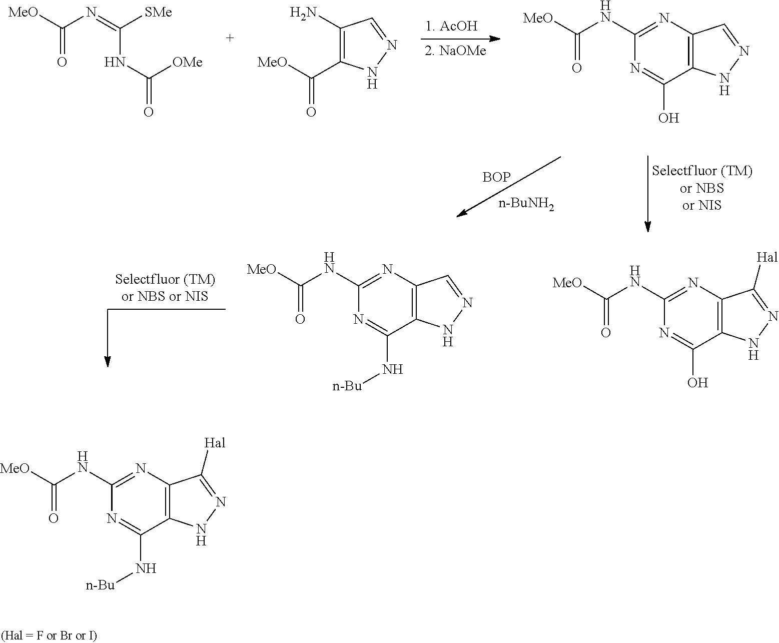

### Biological Activity

The biological activity of compounds disclosed herein as TLR7 agonists can be assayed by the procedures following.

### Human TLR7 Agonist Activity Assay

This procedure describes a method for assaying human TLR7 (hTLR7) agonist activity of the compounds disclosed in this specification.

Engineered human embryonic kidney blue cells (HEK-Blue™ TLR cells; Invivogen) possessing a human TLR7-secreted embryonic alkaline phosphatase (SEAP) reporter transgene were suspended in a non-selective, culture medium (DMEM high-glucose (Invitrogen), supplemented with 10% fetal bovine serum (Sigma)). HEK-Blue™ TLR7 cells were added to each well of a 384-well tissue-culture plate (15,000 cells per well) and incubated 16-18 h at 37° C., 5% CO₂. Compounds (100 nl) were dispensed into wells containing the HEK-Blue™ TLR cells and the treated cells were incubated at 37° C., 5% CO₂. After 18 h treatment ten microliters of freshly-prepared Quanti-Blue” reagent (Invivogen) was added to each well, incubated for 30 min (37° C., 5% CO₂) and SEAP levels measured using an Envision plate reader (OD=620 nm). The half maximal effective concentration values (EC₅₀; compound concentration which induced a response halfway between the assay baseline and maximum) were calculated.

### Induction of Type I Interferon Genes (MX-1) and CD69 in Human Blood

The induction of Type I interferon (IFN) MX-1 genes and the B-cell activation marker CD69 are downstream events that occur upon activation of the TLR7 pathway. The following is a human whole blood assay that measures their induction in response to a TLR7 agonist.

Heparinized human whole blood was harvested from human subjects and treated with test TLR7 agonist compounds at 1 mM. The blood was diluted with RPMI 1640 media and Echo was used to predot 10 nL per well giving a final concentration of 1 uM (10 nL in 10 uL of blood). After mixing on a shaker for 30 sec, the plates were covered and placed in a 37° C. chamber for o/n=17 hrs. Fixing/lysis buffer was prepared (5×→1× in H₂O, warm at 37° C.; Cat #BD 558049) and kept the perm buffer (on ice) for later use.

For surface markers staining (CD69): prepared surface Abs: 0.045 ul hCD14-FITC (ThermoFisher Cat #MHCD1401)+0.6 ul hCD19-ef450 (ThermoFisher Cat #48-0198-42)+1.5 ul hCD69-PE (cat #BD555531)+0.855 ul FACS buffer. Added 3 ul/well, spin 1000 rpm for 1 min and mixed on shaker for 30 sec, put on ice for 30 mins. Stop stimulation after 30 min with 70 uL of prewarmed 1× fix/lysis buffer and use Feliex mate to resuspend (15 times, change tips for each plate) and incubate at 37 C for 10 min.

Centrifuge at 2000 rpm for 5 min aspirate with HCS plate washer, mix on shaker for 30 sec and then wash with 70 uL in dPBS and pelleted 2×s (2000 rpm for 5 min) and 50 ul wash in FACS buffer pelleted 1×s (2000 rpm for 5 min). Mix on shaker for 30 sec. For Intracellular markers staining (MX-1): Add 50 ul of BD Perm buffer III and mix on shaker for 30 sec. Incubate on ice for 30 min (in the dark). Wash with 50 uL of FACS buffer 2× (spin @2300 rpm×5 min after perm) followed by mixing on shaker for 30 sec. Resuspended in 20 ul of FACS buffer containing MX1 antibody ( )(4812)-Alexa 647: Novus Biologicals #NBP2-43704AF647) 20 ul FACS bf+0.8 ul hIgG+0.04 ul MX-1. Spin 1000 rpm for 1 min, mix on shaker for 30 se and the samples were incubated at RT in the dark for 45 min followed by washing 2×FACS buffer (spin @2300 rpm×5 min after perm). Resuspend 20 ul (35 uL total per well) of FACS buffer and cover with foil and place in 4° C. to read the following day. Plates were read on iQuePlus. The results were loaded into toolset and IC50 curves are generated in curve master. The y-axis 100% is set to 1 uM of resiquimod.

### Induction of TNF-Alpha and Type I IFN Response Genes in Mouse Blood

The induction of TNF-alpha and Type I IFN response genes are downstream events that occur upon activation of the TLR7 pathway. The following is an assay that measures their induction in whole mouse blood in response to a TLR7 agonist.

Heparinized mouse whole blood was diluted with RPMI 1640 media with Pen-Strep in the ratio of 5:4 (50 uL whole blood and 40 uL of media). A volume of 90 uL of the diluted blood was transferred to wells of Falcon flat bottom 96-well tissue culture plates, and the plates were incubated at 4° C. for 1 h. Test compounds in 100% DMSO stocks were diluted 20-fold in the same media for concentration response assays, and then 10 uL of the diluted test compounds were added to the wells, so that the final DMSO concentration was 0.5%. Control wells received 10 uL media containing 5% DMSO. The plates were then incubated at 37° C. in a 5% Co₂ incubator for 17 h. Following the incubation, 100 uL of the culture medium as added to each well. The plates were centrifuged and 130 uL of supernatant was removed for use in assays of TNFa production by ELISA (Invitrogen, Catalog Number 88-7324 by Thermo-Fisher Scientific). A 70 uL volume of mRNA catcher lysis buffer (1×) with DTT from the Invitrogen mRNA Catcher Plus kit (Cat #K1570-02) was added to the remaining 70 uL sample in the well, and was mixed by pipetting up and down 5 times. The plate was then shaken at RT for 5-10 min, followed by addition of 2 uL of proteinase K (20 mg/mL) to each well. Plates were then shaken for 15-20 min at RT. The plates were then stored at −80° C. until further processing.

The frozen samples were thawed and mRNA was extracted using the Invitrogen mRNA Catcher Plus kit (Cat #K1570-02) according to the manufacturer's instructions. Half yield of mRNA from RNA extraction were used to synthesize cDNA in 20 μL reverse transcriptase reactions using Invitrogen SuperScript IV VILO Master Mix (Cat #11756500). TaqMan® real-time PCR was performed using QuantStudio Real-Time PCR system from ThermoFisher (Applied Biosystems). All real-time PCR reactions were run in duplicate using commercial predesigned TaqMan assays for mouse IFIT1, IFIT3, MX1 and PPIA gene expression and TaqMan Master Mix. PPIA was utilized as the housekeeping gene. The recommendations from the manufacturer were followed. All raw data (Ct) were normalized by average housekeeping gene (Ct) and then the comparative Ct (ΔΔCt) method were utilized to quantify relative gene expression (RQ) for experimental analysis.

### Definitions

“Aliphatic” means a straight- or branched-chain, saturated or unsaturated, non-aromatic hydrocarbon moiety having the specified number of carbon atoms (e.g., as in “C₃ aliphatic,” “C₁₋₅ aliphatic,” “C₁-C₅ aliphatic,” or “C₁ to C₅ aliphatic,” the latter three phrases being synonymous for an aliphatic moiety having from 1 to 5 carbon atoms) or, where the number of carbon atoms is not explicitly specified, from 1 to 4 carbon atoms (2 to 4 carbons in the instance of unsaturated aliphatic moieties). A similar understanding is applied to the number of carbons in other types, as in C₂-4 alkene, C₄-C₇ cycloaliphatic, etc. In a similar vein, a term such as “(CH₂)₁₋₃” is to be understand as shorthand for the subscript being 1, 2, or 3, so that such term represents CH₂, CH₂CH₂, and CH₂CH₂CH₂.

“Alkyl” means a saturated aliphatic moiety, with the same convention for designating the number of carbon atoms being applicable. By way of illustration, C₁-C₄ alkyl moieties include, but are not limited to, methyl, ethyl, propyl, isopropyl, isobutyl, t-butyl, 1-butyl, 2-butyl, and the like. “Alkanediyl” (sometimes also referred to as “alkylene”) means a divalent counterpart of an alkyl group, such as

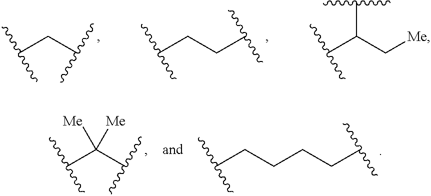

“Alkenyl” means an aliphatic moiety having at least one carbon-carbon double bond, with the same convention for designating the number of carbon atoms being applicable. By way of illustration, C₂-C₄ alkenyl moieties include, but are not limited to, ethenyl (vinyl), 2-propenyl (allyl or prop-2-enyl), cis-1-propenyl, trans-1-propenyl, E- (or Z-) 2-butenyl, 3-butenyl, 1,3-butadienyl (but-1,3-dienyl) and the like.

“Alkynyl” means an aliphatic moiety having at least one carbon-carbon triple bond, with the same convention for designating the number of carbon atoms being applicable. By way of illustration, C₂-C₄ alkynyl groups include ethynyl (acetylenyl), propargyl (prop-2-ynyl), 1-propynyl, but-2-ynyl, and the like.

“Cycloaliphatic” means a saturated or unsaturated, non-aromatic hydrocarbon moiety having from 1 to 3 rings, each ring having from 3 to 8 (preferably from 3 to 6) carbon atoms. “Cycloalkyl” means a cycloaliphatic moiety in which each ring is saturated. “Cyclo-alkenyl” means a cycloaliphatic moiety in which at least one ring has at least one carbon-carbon double bond. “Cycloalkynyl” means a cycloaliphatic moiety in which at least one ring has at least one carbon-carbon triple bond. By way of illustration, cycloaliphatic moieties include, but are not limited to, cyclopropyl, cyclobutyl, cyclopentyl, cyclopentenyl, cyclohexyl, cyclohexenyl, cycloheptyl, cyclooctyl, and adamantyl. Preferred cycloaliphatic moieties are cycloalkyl ones, especially cyclopropyl, cyclobutyl, cyclopentyl, and cyclohexyl. “Cycloalkanediyl” (sometimes also referred to as “cycloalkylene”) means a divalent counterpart of a cycloalkyl group. Similarly, “bicycloalkanediyl” (osr “bicycloalkylene”) and “spiroalkanediyl” (or “spiroalkylene”) refer to divalent counterparts of a bicycloalkyl and spiroalkyl (or “spirocycloalkyl”) group.

“Heterocycloaliphatic” means a cycloaliphatic moiety wherein, in at least one ring thereof, up to three (preferably 1 to 2) carbons have been replaced with a heteroatom independently selected from N, O, or S, where the N and S optionally may be oxidized and the N optionally may be quaternized. Preferred cycloaliphatic moieties consist of one ring, 5- to 6-membered in size. Similarly, “heterocycloalkyl,” “heterocycloalkenyl,” and “heterocycloalkynyl” means a cycloalkyl, cycloalkenyl, or cycloalkynyl moiety, respectively, in which at least one ring thereof has been so modified. Exemplary heterocycloaliphatic moieties include aziridinyl, azetidinyl, 1,3-dioxanyl, oxetanyl, tetrahydrofuryl, pyrrolidinyl, piperidinyl, piperazinyl, tetrahydropyranyl, tetrahydrothiopyranyl, tetrahydrothiopyranyl sulfone, morpholinyl, thiomorpholinyl, thiomorpholinyl sulfoxide, thiomorpholinyl sulfone, 1,3-dioxolanyl, tetrahydro-1,1-dioxothienyl, 1,4-dioxanyl, thietanyl, and the like. “Heterocycloalkylene” means a divalent counterpart of a heterocycloalkyl group.

“Alkoxy,” “aryloxy,” “alkylthio,” and “arylthio” mean —O(alkyl), —O(aryl), —S(alkyl), and —S(aryl), respectively. Examples are methoxy, phenoxy, methylthio, and phenylthio, respectively.

“Halogen” or “halo” means fluorine, chlorine, bromine or iodine, unless a narrower meaning is indicated.

“Aryl” means a hydrocarbon moiety having a mono-, bi-, or tricyclic ring system (preferably monocyclic) wherein each ring has from 3 to 7 carbon atoms and at least one ring is aromatic. The rings in the ring system may be fused to each other (as in naphthyl) or bonded to each other (as in biphenyl) and may be fused or bonded to non-aromatic rings (as in indanyl or cyclohexylphenyl). By way of further illustration, aryl moieties include, but are not limited to, phenyl, naphthyl, tetrahydronaphthyl, indanyl, biphenyl, phenanthryl, anthracenyl, and acenaphthyl. “Arylene” means a divalent counterpart of an aryl group, for example 1,2-phenylene, 1,3-phenylene, or 1,4-phenylene.

“Heteroaryl” means a moiety having a mono-, bi-, or tricyclic ring system (preferably 5- to 7-membered monocyclic) wherein each ring has from 3 to 7 carbon atoms and at least one ring is an aromatic ring containing from 1 to 4 heteroatoms independently selected from N, O, or S, where the N and S optionally may be oxidized and the N optionally may be quaternized. Such at least one heteroatom containing aromatic ring may be fused to other types of rings (as in benzofuranyl or tetrahydroisoquinolyl) or directly bonded to other types of rings (as in phenylpyridyl or 2-cyclopentylpyridyl). By way of further illustration, heteroaryl moieties include pyrrolyl, furanyl, thiophenyl (thienyl), imidazolyl, pyrazolyl, oxazolyl, isoxazolyl, thiazolyl, isothiazolyl, triazolyl, tetrazolyl, pyridyl, N-oxopyridyl, pyridazinyl, pyrimidinyl, pyrazinyl, quinolinyl, isoquinolynyl, quinazolinyl, cinnolinyl, quinozalinyl, naphthyridinyl, benzofuranyl, indolyl, benzothiophenyl, oxadiazolyl, thiadiazolyl, phenothiazolyl, benzimidazolyl, benzotriazolyl, dibenzofuranyl, carbazolyl, dibenzothiophenyl, acridinyl, and the like. “Heteroarylene” means a divalent counterpart of a heteroaryl group.

Where it is indicated that a moiety may be substituted, such as by use of “unsubstituted or substituted” or “optionally substituted” phrasing as in “unsubstituted or substituted C₁-C₅ alkyl” or “optionally substituted heteroaryl,” such moiety may have one or more independently selected substituents, preferably one to five in number, more preferably one or two in number. Substituents and substitution patterns can be selected by one of ordinary skill in the art, having regard for the moiety to which the substituent is attached, to provide compounds that are chemically stable and that can be synthesized by techniques known in the art as well as the methods set forth herein. Where a moiety is identified as being “unsubstituted or substituted” or “optionally substituted,” in a preferred embodiment such moiety is unsubstituted.

“Arylalkyl,” (heterocycloaliphatic)alkyl,” “arylalkenyl,” “arylalkynyl,” “biarylalkyl,” and the like mean an alkyl, alkenyl, or alkynyl moiety, as the case may be, substituted with an aryl, heterocycloaliphatic, biaryl, etc., moiety, as the case may be, with the open (unsatisfied) valence at the alkyl, alkenyl, or alkynyl moiety, for example as in benzyl, phenethyl, N-imidazoylethyl, N-morpholinoethyl, and the like. Conversely, “alkylaryl,” “alkenylcycloalkyl,” and the like mean an aryl, cycloalkyl, etc., moiety, as the case may be, substituted with an alkyl, alkenyl, etc., moiety, as the case may be, for example as in methylphenyl (tolyl) or allylcyclohexyl. “Hydroxyalkyl,” “haloalkyl,” “alkylaryl,” “cyanoaryl,” and the like mean an alkyl, aryl, etc., moiety, as the case may be, substituted with one or more of the identified substituent (hydroxyl, halo, etc., as the case may be).

For example, permissible substituents include, but are not limited to, alkyl (especially methyl or ethyl), alkenyl (especially allyl), alkynyl, aryl, heteroaryl, cycloaliphatic, heterocycloaliphatic, halo (especially fluoro), haloalkyl (especially trifluoromethyl), hydroxyl, hydroxyalkyl (especially hydroxyethyl), cyano, nitro, alkoxy, —O(hydroxyalkyl), —O(haloalkyl) (especially —OCF₃), —O(cycloalkyl), —O(heterocycloalkyl), —O(aryl), alkylthio, arylthio, ═O, ═NH, ═N(alkyl), ═NOH, ═NO(alkyl), —C(═O)(alkyl), —C(═O)H, —CO₂H, —C(═O)NHOH, —C(═O)O(alkyl), —C(═O)O(hydroxyalkyl), —C(═O)NH₂, —C(═O)NH(alkyl), —C(═O)N(alkyl)₂, —OC(═O)(alkyl), —OC(═O)(hydroxyalkyl), —OC(═O)O(alkyl), —OC(═O)O(hydroxyalkyl), —OC(═O)NH₂, —OC(═O)NH(alkyl), —OC(═O)N(alkyl)₂, azido, —NH₂, —NH(alkyl), —N(alkyl)₂, —NH(aryl), —NH(hydroxyalkyl), —NHC(═O)(alkyl), —NHC(═O)H, —NHC(═O)NH₂, —NHC(═O)NH(alkyl), —NHC(═O)N(alkyl)₂, —NHC(═NH)NH₂, —OSO₂(alkyl), —SH, —S(alkyl), —S(aryl), —S(cycloalkyl), —S(═O)alkyl, —SO₂(alkyl), —SO₂NH₂, —SO₂NH(alkyl), —SO₂N(alkyl)₂, and the like.

Where the moiety being substituted is an aliphatic moiety, preferred substituents are aryl, heteroaryl, cycloaliphatic, heterocycloaliphatic, halo, hydroxyl, cyano, nitro, alkoxy, —O(hydroxyalkyl), —O(haloalkyl), —O(cycloalkyl), —O(heterocycloalkyl), —O(aryl), alkylthio, arylthio, ═O, ═NH, ═N(alkyl), ═NOH, ═NO(alkyl), —CO₂H, —C(═O)NHOH, —C(═O)O(alkyl), —C(═O)O(hydroxyalkyl), —C(═O)NH₂, —C(═O)NH(alkyl), —C(═O)N(alkyl)₂, —OC(═O)(alkyl), —OC(═O)(hydroxyalkyl), —OC(═O)O(alkyl), —OC(═O)O(hydroxyalkyl), —OC(═O)NH₂, —OC(═O)NH(alkyl), —OC(═O)N(alkyl)₂, azido, —NH₂, —NH(alkyl), —N(alkyl)₂, —NH(aryl), —NH(hydroxyalkyl), —NHC(═O)(alkyl), —NHC(═O)H, —NHC(═O)NH₂, —NHC(═O)NH(alkyl), —NHC(═O)N(alkyl)₂, —NHC(═NH)NH₂, —OSO₂(alkyl), —SH, —S(alkyl), —S(aryl), —S(═O)alkyl, —S(cycloalkyl), —SO₂(alkyl), —SO₂NH₂, —SO₂NH(alkyl), and —SO₂N(alkyl)₂. More preferred substituents are halo, hydroxyl, cyano, nitro, alkoxy, —O(aryl), ═O, ═NOH, ═NO(alkyl), —OC(═O)(alkyl), —OC(═O)O(alkyl), —OC(═O)NH₂, —OC(═O)NH(alkyl), —OC(═O)N(alkyl)₂, azido, —NH₂, —NH(alkyl), —N(alkyl)₂, —NH(aryl), —NHC(═O)(alkyl), —NHC(═O)H, —NHC(═O)NH₂, —NHC(═O)NH(alkyl), —NHC(═O)N(alkyl)₂, and —NHC(═NH)NH₂. Especially preferred are phenyl, cyano, halo, hydroxyl, nitro, C₁-C₄ alkyoxy, O(C₂-C₄ alkanediyl)OH, and O(C₂-C₄ alkanediyl)halo.

Where the moiety being substituted is a cycloaliphatic, heterocycloaliphatic, aryl, or heteroaryl moiety, preferred substituents are alkyl, alkenyl, alkynyl, halo, haloalkyl, hydroxyl, hydroxyalkyl, cyano, nitro, alkoxy, —O(hydroxyalkyl), —O(haloalkyl), —O(aryl), —O(cycloalkyl), —O(heterocycloalkyl), alkylthio, arylthio, —C(═O)(alkyl), —C(═O)H, —CO₂H, —C(═O)NHOH, —C(═O)O(alkyl), —C(═O)O(hydroxyalkyl), —C(═O)NH₂, —C(═O)NH(alkyl), —C(═O)N(alkyl)₂, —OC(═O)(alkyl), —OC(═O)(hydroxyalkyl), —OC(═O)O(alkyl), —OC(═O)O(hydroxyalkyl), —OC(═O)NH₂, —OC(═O)NH(alkyl), —OC(═O)N(alkyl)₂, azido, —NH₂, —NH(alkyl), —N(alkyl)₂, —NH(aryl), —NH(hydroxyalkyl), —NHC(═O)(alkyl), —NHC(═O)H, —NHC(═O)NH₂, —NHC(═O)NH(alkyl), —NHC(═O)N(alkyl)₂, —NHC(═NH)NH₂, —OSO₂(alkyl), —SH, —S(alkyl), —S(aryl), —S(cycloalkyl), —S(═O)alkyl, —SO₂(alkyl), —SO₂NH₂, —SO₂NH(alkyl), and —SO₂N(alkyl)₂. More preferred substituents are alkyl, alkenyl, halo, haloalkyl, hydroxyl, hydroxyalkyl, cyano, nitro, alkoxy, —O(hydroxyalkyl), —C(═O)(alkyl), —C(═O)H, —CO₂H, —C(═O)NHOH, —C(═O)O(alkyl), —C(═O)O(hydroxyalkyl), —C(═O)NH₂, —C(═O)NH(alkyl), —C(═O)N(alkyl)₂, —OC(═O)(alkyl), —OC(═O)(hydroxyalkyl), —OC(═O)O(alkyl), —OC(═O)O(hydroxyalkyl), —OC(═O)NH₂, —OC(═O)NH(alkyl), —OC(═O)N(alkyl)₂, —NH₂, —NH(alkyl), —N(alkyl)₂, —NH(aryl), —NHC(═O)(alkyl), —NHC(═O)H, —NHC(═O)NH₂, —NHC(═O)NH(alkyl), —NHC(═O)N(alkyl)₂, and —NHC(═NH)NH₂. Especially preferred are C₁-C₄ alkyl, cyano, nitro, halo, and C₁-C₄alkoxy.

Where a range is stated, as in “C₁-C₅ alkyl” or “5 to 10%,” such range includes the end points of the range, as in C₁ and C₅ in the first instance and 5% and 10% in the second instance.

Unless particular stereoisomers are specifically indicated (e.g., by a bolded or dashed bond at a relevant stereocenter in a structural formula, by depiction of a double bond as having E or Z configuration in a structural formula, or by use stereochemistry-designating nomenclature or symbols), all stereoisomers are included within the scope of the invention, as pure compounds as well as mixtures thereof. Unless otherwise indicated, racemates, individual enantiomers (whether optically pure or partially resolved), diastereomers, geometrical isomers, and combinations and mixtures thereof are all encompassed by this invention.

Those skilled in the art will appreciate that compounds may have tautomeric forms (e.g., keto and enol forms), resonance forms, and zwitterionic forms that are equivalent to those depicted in the structural formulae used herein and that the structural formulae encompass such tautomeric, resonance, or zwitterionic forms.

“Pharmaceutically acceptable ester” means an ester that hydrolyzes in vivo (for example in the human body) to produce the parent compound or a salt thereof or has perse activity similar to that of the parent compound. Suitable esters include C₁-C₅ alkyl, C₂-C₅ alkenyl or C₂-C₅ alkynyl esters, especially methyl, ethyl or n-propyl.

“Pharmaceutically acceptable salt” means a salt of a compound suitable for pharmaceutical formulation. Where a compound has one or more basic groups, the salt can be an acid addition salt, such as a sulfate, hydrobromide, tartrate, mesylate, maleate, citrate, phosphate, acetate, pamoate (embonate), hydroiodide, nitrate, hydrochloride, lactate, methyl-sulfate, fumarate, benzoate, succinate, mesylate, lactobionate, suberate, tosylate, and the like. Where a compound has one or more acidic groups, the salt can be a salt such as a calcium salt, potassium salt, magnesium salt, meglumine salt, ammonium salt, zinc salt, piperazine salt, tromethamine salt, lithium salt, choline salt, diethylamine salt, 4-phenylcyclohexylamine salt, benzathine salt, sodium salt, tetramethylammonium salt, and the like. Polymorphic crystalline forms and solvates are also encompassed within the scope of this invention.

“Subject” refers to an animal, including, but not limited to, a primate (e.g., human), monkey, cow, pig, sheep, goat, horse, dog, cat, rabbit, rat, or mouse. The terms “subject” and “patient” are used interchangeably herein in reference, for example, to a mammalian subject, such as a human.

The terms “treat,” “treating,” and “treatment,” in the context of treating a disease or disorder, are meant to include alleviating or abrogating a disorder, disease, or condition, or one or more of the symptoms associated with the disorder, disease, or condition; or to slowing the progression, spread or worsening of a disease, disorder or condition or of one or more symptoms thereof. The “treatment of cancer”, refers to one or more of the following effects: (1) inhibition, to some extent, of tumor growth, including, (i) slowing down and (ii) complete growth arrest; (2) reduction in the number of tumor cells; (3) maintaining tumor size; (4) reduction in tumor size; (5) inhibition, including (i) reduction, (ii) slowing down or (iii) complete prevention, of tumor cell infiltration into peripheral organs; (6) inhibition, including (i) reduction, (ii) slowing down or (iii) complete prevention, of metastasis; (7) enhancement of anti-tumor immune response, which may result in (i) maintaining tumor size, (ii) reducing tumor size, (iii) slowing the growth of a tumor, (iv) reducing, slowing or preventing invasion and/or (8) relief, to some extent, of the severity or number of one or more symptoms associated with the disorder.

In the formulae of this specification, a wavy line ( [symbol] ) transverse to a bond or an asterisk (*) at the end of the bond denotes a covalent attachment site. For instance, a statement that R is

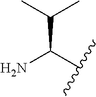 [SMILES: *[C@@H](N)C(C)C]

or that R is

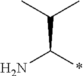 [SMILES: *[C@@H](N)C(C)C]

in the formula

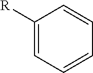

means

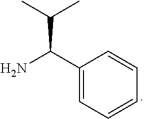

In the formulae of this specification, a bond traversing an aromatic ring between two carbons thereof means that the group attached to the bond may be located at any of the positions of the aromatic ring made available by removal of the hydrogen that is implicitly there (or explicitly there, if written out). By way of illustration:

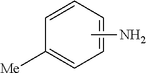

represents

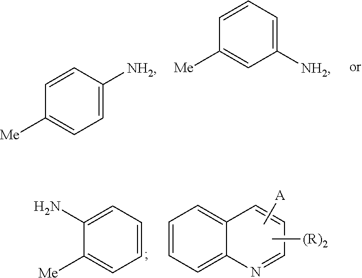

represents

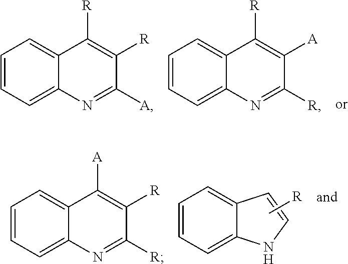

represents

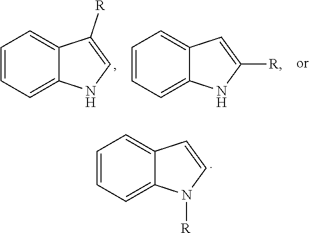

This disclosure includes all isotopes of atoms occurring in the compounds described herein. Isotopes include those atoms having the same atomic number but different mass numbers. By way of general example and without limitation, isotopes of hydrogen include deuterium and tritium. Isotopes of carbon include ¹³C and ¹⁴C. Isotopically-labeled compounds of the invention can generally be prepared by conventional techniques known to those skilled in the art or by processes analogous to those described herein, using an appropriate isotopically-labeled reagent in place of the non-labeled reagent otherwise employed. By way of example, a C₁-C₃ alkyl group can be undeuterated, partially deuterated, or fully deuterated and “CH₃” includes CH₃, ¹³CH₃, ¹⁴CH₃, CH₂T, CH₂D, CHD₂, CD₃, etc. In one embodiment, the various elements in a compound are present in their natural isotopic abundance.

Those skilled in the art will appreciate that certain structures can be drawn in one tautomeric form or another—for example, keto versus enol—and that the two forms are equivalent.

### ACRONYMS AND ABBREVIATIONS

Table C provides a list of acronyms and abbreviations used in this specification, along with their meanings.

TABLE C

| ACRONYM OR | |
|---|---|
| ABBREVIATION | MEANING OR DEFINITION |
| AIBN | Azobisisobutyronitrile |
| Alloc | Allyloxycarbonyl |
| Aq. | Aqueous |
| Boc | t-Butyloxycarbonyl |
| BOP | (Benzotriazol-1-yloxy)tris(dimethylamino)- |
| | phosphonium hexafluorophosphate (V) |
| BOP | (Benzotriazol-1-yloxy)tris(dimethylamino)- |
| | phosphonium hexafluorophosphate (V) |
| DBU | 1,8-Diazabicyclo[5.4.0]undec-7-ene |
| DCM | Dichloromethane |
| DIAD | Diisopropyl azodicarboxylate |
| DIPEA, DIEA | N,N-diisopropylethylamine, also known as |
| | Hünig's base |
| DMA | N,N-Dimethylacetamide |
| DMAP | 4-(Dimethylamino)pyridine |
| DMF | N,N-dimethylformamide |
| DMSO | Dimethyl sulfoxide |
| DTDP | 2,2′-dithiodipyridine |
| DTPA | Diethylenetriaminepentaacetic acid |
| EEDQ | Ethyl 2-ethoxyquinoline-1(2H)-carboxylate |
| Fmoc | Fluorenylmethyloxycarbonyl |
| HATU | Hexafluorophosphate Azabenzotriazole |
| | Tetramethyl Uronium; 1-[Bis(dimeth- |
| | ylamino)-methylene]-1H-1,2,3- |
| | triazolo[4,5-b]pyridinium 3- |
| | oxide hexafluorophosphate |
| HEPES | 4-(2-Hydroxyethyl)piperazine-1- |
| | ethanesulfonic acid, N-(2- |
| | Hydroxyethyl)piperazine-N′-(2- |
| | ethanesulfonic acid) |
| HPLC | High pressure liquid chromatography |
| Hunig's base | See DIPEA, DIEA |
| LCMS, LC-MS, LC/MS | Liquid chromatography-mass spectrometry |
| mCPBA | m-chloroperbenzoic acid |
| MS | Mass spectrometry |
| MsCl | Methanesylfonyl chloride, mesyl chloride |
| NBS | N-Bromosuccinimide |
| NMR | Nuclear magnetic resonance |
| PEG | Poly(ethylene glycol) |
| PTFE | Poly(tetrafluoroethylene) |
| RT (in context of liquid | Retention time, in min |
| chromatography) | |
| RT (in the context of | Room (ambient) temperature, circa 25° C. |
| reaction conditions) | |
| Sat. | Saturated |
| Soln | Solution |
| TBDPS | tert-Butyldiphenylsilyl |
| TBS | t-Butyldimethylsilyl group |
| TEA | Triethylamine |
| TEAA | Triethylammonium acetate |
| TFA | Trifluoroacetic acid |
| THF | Tetrahydrofuran |

### REFERENCES

Full citations for the following references cited in abbreviated fashion by first author (or inventor) and date earlier in this specification are provided below. Each of these references is incorporated herein by reference for all purposes.
Akinbobuyi et al., Tetrahedron Lett. 2015, 56, 458, “Facile syntheses of functionalized toll-like receptor 7 agonists”.
Akinbobuyi et al., Bioorg. Med. Chem. Lett. 2016, 26, 4246, “Synthesis and immunostimulatory activity of substituted TLR7 agonists.”
Barberis et al., US 2012/0003298 A1 (2012).
Beesu et al., J. Med. Chem. 2017, 60, 2084, “Identification of High-Potency Human TLR8 and Dual TLR7/TLR8 Agonists in Pyrimidine-2,4-diamines.”
Berghöfer et al., J. Immunol. 2007, 178, 4072, “Natural and Synthetic TLR7 Ligands Inhibit CpG-A- and CpG-C-Oligodeoxynucleotide-Induced IFN-α Production.”
Bonfanti et al., US 2014/0323441 A1 (2015) [2015a].
Bonfanti et al., US 2015/0299221 A1 (2015) [2015b].
Bonfanti et al., US 2016/0304531 A1 (2016).
Carson et al., US 2013/0202629 A1 (2013).
Carson et al., U.S. Pat. No. 8,729,088 B2 (2014).
Carson et al., U.S. Pat. No. 9,050,376 B2 (2015).
Carson et al., US 2016/0199499 A1 (2016).
Chan et al., Bioconjugate Chem. 2009, 20, 1194, “Synthesis and Immunological Characterization of Toll-Like Receptor 7 Agonistic Conjugates.”
Chan et al., Bioconjugate Chem. 2011, 22, 445, “Synthesis and Characterization of PEGylated Toll Like Receptor 7 Ligands.”
Chen et al., U.S. Pat. No. 7,919,498 B2 (2011).
Coe et al., U.S. Pat. No. 9,662,336 B2 (2017).
Cortez and Va, Medicinal Chem. Rev. 2018, 53, 481, “Recent Advances in Small-Molecule TLR7 Agonists for Drug Discovery”.
Cortez et al., US 2017/0121421 A1 (2017).
Cortez et al., U.S. Pat. No. 9,944,649 B2 (2018).
Dellaria et al., WO 2007/028129 A1 (2007).
Desai et al., U.S. Pat. No. 9,127,006 B2 (2015).
Ding et al., WO 2016/107536 A1 (2016).
Ding et al., US 2017/0273983 A1 (2017) [2017a].
Ding et al., WO 2017/076346 A1 (2017) [2017b].
Gadd et al., Bioconjugate Chem. 2015, 26, 1743, “Targeted Activation of Toll-Like Receptors: Conjugation of a Toll-Like Receptor 7 Agonist to a Monoclonal Antibody Maintains Antigen Binding and Specificity.”
Graupe et al., U.S. Pat. No. 8,993,755 B2 (2015).
Embrechts et al., J. Med. Chem. 2018, 61, 6236, “2,4-Diaminoquinazolines as Dual Toll Like Receptor (TLR) 7/8 Modulators for the Treatment of Hepatitis B Virus.”
Halcomb et al., U.S. Pat. No. 9,161,934 B2 (2015).
Hashimoto et al., US 2009/0118263 A1 (2009).
He et al., U.S. Pat. No. 10,487,084 B2 (2019) [2019a].
He et al., U.S. Pat. No. 10,508,115 B2 (2019) [2019b].
Hirota et al., U.S. Pat. No. 6,028,076 (2000).
Holldack et al., US 2012/0083473 A1 (2012).
Isobe et al., U.S. Pat. No. 6,376,501 B1 (2002).
Isobe et al., JP 2004137157 (2004).
Isobe et al., J. Med. Chem. 2006, 49 (6), 2088, “Synthesis and Biological Evaluation of Novel 9-Substituted-8-Hydroxyadenine Derivatives as Potent Interferon Inducers.”
Isobe et al., U.S. Pat. No. 7,521,454 B2 (2009) [2009a].
Isobe et al., US 2009/0105212 A1 (2009) [2009b].
Isobe et al., US 2011/0028715 A1 (2011).
Isobe et al., U.S. Pat. No. 8,148,371 B2 (2012).
Jensen et al., WO 2015/036044 A1 (2015).
Jones et al., U.S. Pat. No. 7,691,877 B2 (2010).
Jones et al., US 2012/0302598 A1 (2012).
Kasibhatla et al., U.S. Pat. No. 7,241,890 B2 (2007).
Koga-Yamakawa et al., Int. J. Cancer 2013, 132 (3), 580, “Intratracheal and oral administration of SM-276001: A selective TLR7 agonist, leads to antitumor efficacy in primary and metastatic models of cancer.”
Li et al., U.S. Pat. No. 9,902,730 B2 (2018).
Lioux et al., U.S. Pat. No. 9,295,732 B2 (2016).
Lund et al., Proc. Nat'l Acad. Sci (USA) 2004, 101 (15), 5598, “Recognition of single-stranded RNA viruses by Toll-like receptor 7.”
Maj et al., U.S. Pat. No. 9,173,935 B2 (2015).
McGowan et al., US 2016/0168150 A1 (2016) [2016a].
McGowan et al., U.S. Pat. No. 9,499,549 B2 (2016) [2016b].
McGowan et al., J. Med. Chem. 2017, 60, 6137, “identification and Optimization of Pyrrolo[3,2-d]pyrimidine Toll-like Receptor 7 (TLR7) Selective Agonists for the Treatment of Hepatitis B.”
Musmuca et al., J. Chem. Information & Modeling 2009, 49 (7), 1777, “Small-is Molecule Interferon Inducers. Toward the Comprehension of the Molecular Determinants through Ligand-Based Approaches.”
Nakamura et al., Bioorg. Med. Chem. Lett. 2013, 13, 669, “Synthesis and evaluation of 8-oxoadenine derivatives as potent Toll-like receptor agonists with high water solubility.”
Ogita et al., US 2007/0225303 A1 (2007).
Ota et al., WO 2019/124500 A1 (2019).
Pilatte et al., WO 2017/216293 A1 (2017).
Poudel et al., U.S. Pat. No. 10,472,361 B2 (2019) [2019a].
Poudel et al., U.S. Pat. No. 10,494,370 B2 (2019) [2019b].
Poudel et al., US 2020/0038403 A1 (2020) [2020a].
Poudel et al., US 2020/0039984 A1 (2020) [2020b].
Purandare et al., WO 2019/209811 A1 (2019).
Pryde, U.S. Pat. No. 7,642,350 B2 (2010).
Sato-Kaneko et al., JCi Insight 2017, 2, e93397, “Combination Immunotherapy with TLR Agonists and Checkpoint Inhibitors Suppresses Head and Neck Cancer”.
Smits et al., The Oncologist 2008, 13, 859, “The Use of TLR7 and TLR8 Ligands for the Enhancement of Cancer Immunotherapy”.
Vasilakos and Tomai, Expert Rev. Vaccines 2013, 12, 809, “The Use of Toll-like Receptor 7/8 Agonists as Vaccine Adjuvants”.
Vernejoul et al., U.S. Pat. No. 10,457,681 B2 (2014).
Young et al., US 2019/0055244 A1 (2019).
Yu et al., PLoS One 2013, 8 (3), e56514, “Toll-Like Receptor 7 Agonists: Chemical Feature Based Pharmacophore Identification and Molecular Docking Studies.”
Zhang et al., Immunity 2016, 45, 737, “Structural Analysis Reveals that Toll-like Receptor 7 Is a Dual Receptor for Guanosine and Single-Stranded RNA.”
Zhang et al., WO 2018/095426 A1 (2018)>
Zurawski et al., US 2012/0231023 A1 (2012).

The foregoing detailed description of the invention includes passages that are chiefly or exclusively concerned with particular parts or aspects of the invention. It is to be understood that this is for clarity and convenience, that a particular feature may be relevant in more than just the passage in which it is disclosed, and that the disclosure herein includes all the appropriate combinations of information found in the different passages. Similarly, although the various figures and descriptions herein relate to specific embodiments of the invention, it is to be understood that where a specific feature is disclosed in the context of a particular figure or embodiment, such feature can also be used, to the extent appropriate, in the context of another figure or embodiment, in combination with another feature, or in the invention in general.

Further, while the present invention has been particularly described in terms of certain preferred embodiments, the invention is not limited to such preferred embodiments. Rather, the scope of the invention is defined by the appended claims.

1. A compound having a structure according to formula I or formula (II) 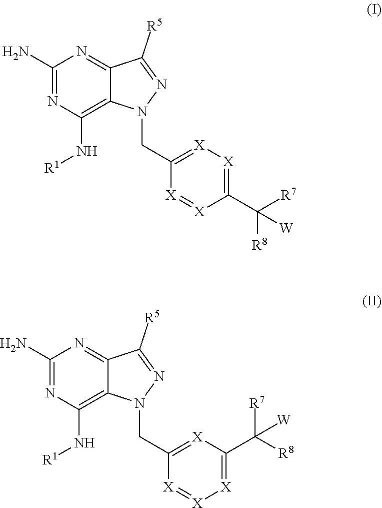
wherein
each X is independently N or CR²;
W is R³ or 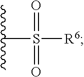
R¹ is (C₁-C₅ alkyl),
(C₂-C₅ alkenyl),
(C₁-C₈ alkanediyl)₀₋₁(C₃-C₆ cycloalkyl),
(C₁-C₈ alkanediyl)₀₋₁(C₅-C₁₀ spiroalkyl),
(C₂-C₈ alkanediyl)OH,
(C₂-C₈ alkanediyl)O(C₁-C₃ alkyl),
(C₁-C₄ alkanediyl)₀₋₁(5-6 membered heteroaryl),
(C₁-C₄ alkanediyl)₀₋₁phenyl,
(C₁-C₄ alkanediyl)CF₃,
(C₂-C₈ alkanediyl)N[C(═O)](C₁-C₃ alkyl),
or
(C₂-C₈ alkanediyl)NR^{x}R^{y};
each R² is independently H, O(C₁-C₃ alkyl), S(C₁-C₃ alkyl), SO₂(C₁-C₃ alkyl), C₁-C₃ alkyl, O(C₃-C₄ cycloalkyl), S(C₃-C₄ cycloalkyl), SO₂(C₃-C₄ cycloalkyl), C₃-C₄ cycloalkyl, Cl, F, CN; or [C(═O)]₀₋₁NR^{x}R^{y};
R³ is NH₂,
NH[C(═O)]₀₋₁(C₁-C₅ alkyl),
N(C₁-C₅ alkyl)₂,
NH[C(═O)]₀₋₁(C₁-C₄ alkanediyl)₀₋₁(C₃-C₈ cycloalkyl),
NH[C(═O)]₀₋₁(C₁-C₄ alkanediyl)₀₋₁(C₄-C₁₀ bicycloalkyl),
NH[C(═O)]₀₋₁(C₁-C₄ alkanediyl)₀₋₁(C₅-C₁₀ spiroalkyl),
N(C₃-C₆ cycloalkyl)₂,
N[C₁-C₃ alkyl]C(═O)(C₁-C₆ alkyl),
NH(SO₂)(C₁-C₈ alkyl),
NH(SO₂)(C₁-C₄ alkanediyl)₀₋₁(C₃-C₈ cycloalkyl),
NH(SO₂)(C₁-C₄ alkanediyl)₀₋₁(C₄-C₁₀ bicycloalkyl),
NH(SO₂)(C₁-C₄ alkanediyl)₀₋₁(C₅-C₁₀ spiroalkyl),
a 6-membered aromatic or heteroaromatic moiety,
a 5-membered heteroaromatic moiety, or
a moiety having the structure 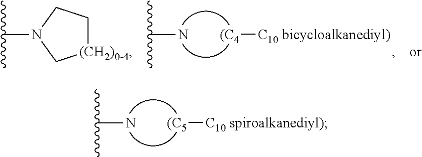
R⁵ is H, C₁-C₅ alkyl, C₂-C₅ alkenyl, C₃-C₆ cycloalkyl, halo, O(C₁-C₅ alkyl), (C₁-C₄ alkanediyl)OH, (C₁-C₄ alkanediyl)O(C₁-C₃ alkyl), phenyl, NH(C₁-C₅ alkyl), 5 or 6 membered heteroaryl, 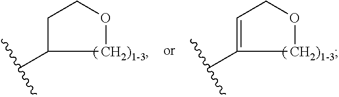
R⁶ is NH₂,
(NH)₀₋₁(C₁-C₈ alkyl),
N(C₁-C₅ alkyl)₂,
(NH)₀₋₁(C₁-C₄ alkanediyl)₀₋₁(C₃-C₈ cycloalkyl),
(NH)₀₋₁(C₁-C₄ alkanediyl)₀₋₁(C₄-C₁₀ bicycloalkyl),
(NH)₀₋₁(C₁-C₄ alkanediyl)₀₋₁(C₅-C₁₀ spiroalkyl),
N(C₃-C₆ cycloalkyl)₂,
or
a moiety having the structure 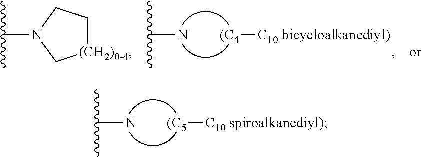
R⁷ and R⁸ are independently
C₁-C₄ alkyl,
C₂-C₄ alkylene,
C₃-C₄ cycloalkyl,
or R⁷ and R⁸ combine with the carbon to which they are bonded to form a 3- to 7-membered cycloalkyl moiety;
R^{x} and R^{y} are independently H or C₁-C₃ alkyl or R^{x} and R^{y} combine with the nitrogen to which they are bonded to form a 3- to 7-membered heterocycle;
wherein in R¹, R², R³, R⁵, R⁶, R⁷, and R⁸
an alkyl moiety, alkanediyl moiety, cycloalkyl moiety, or a moiety of the formula 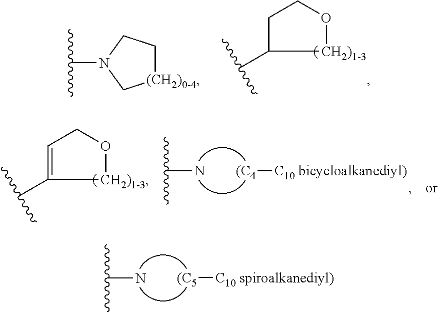
is optionally substituted with one or more substituents selected from OH, halo, CN, (C₁-C₃ alkyl), O(C₁-C₃ alkyl), C(═O)(C₁-C₃ alkyl), SO₂(C₁-C₃ alkyl), NR^{x}R^{y}, (C₁-C₄ alkanediyl)OH, (C₁-C₄ alkanediyl)O(C₁-C₃ alkyl);
and
an alkyl, alkanediyl, cycloalkyl, or moiety of the formula 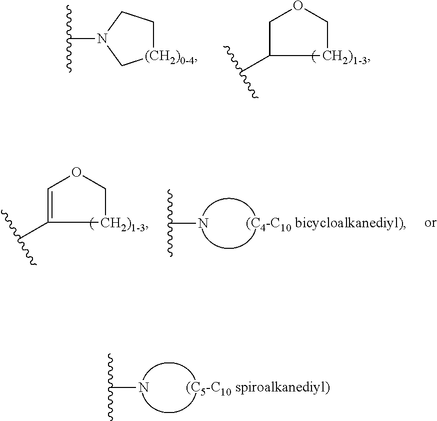
may have a CH₂ group replaced by O, SO₂, CF₂, C(═O), NH,
N[C(═O)]₀₋₁(C₁-C₃ alkyl),
N[C(═O)]₀₋₁(C₁-C₄ alkanediyl)CF₃,
N[C(═O)]₀₋₁(C₁-C₄ alkanediyl)OH,
or
N[C(═O)]₀₋₁(C₁-C₄ alkanediyl)₀₋₁(C₃-C₅ cycloalkyl).

2. A compound according to claim 1, wherein R¹ is selected from the group consisting of

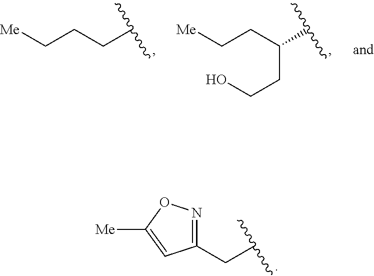 [SMILES: *CCCC ; *[C@@H](CCC)CCO ; *Cc1cc(C)on1]

3. A compound according to claim 1, wherein R² is OMe.

4. A compound according to claim 1, wherein R⁵ is H.

5. A compound according to claim 1, having a structure according to formula (Ia)

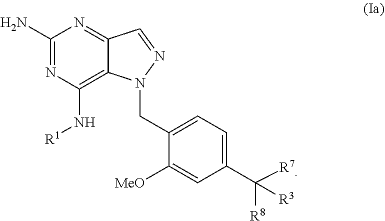

6. A compound according to claim 5, wherein

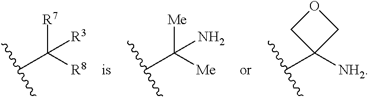

7. A compound according to claim 1, having a structure according to formula (IIa)

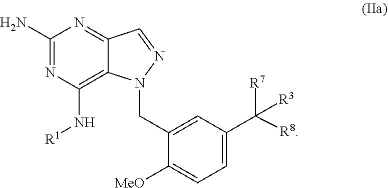

8. A compound according to claim 7, wherein

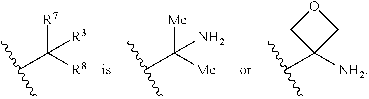

9. A compound according to formula (Ia) or (IIa) 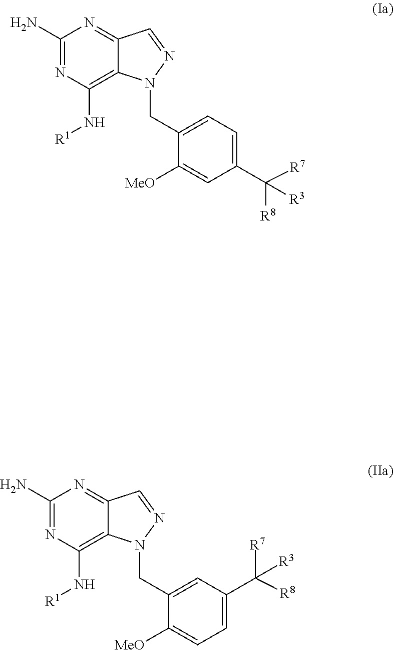
wherein
R¹ is

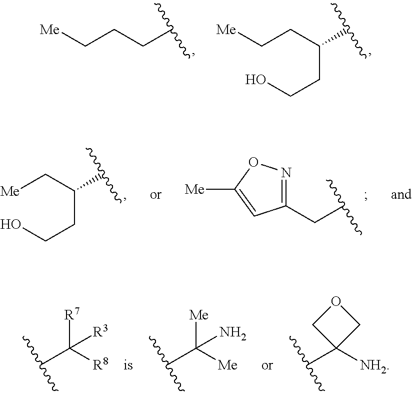

10. A method of treating a cancer, comprising administering to a patient suffering from such cancer a therapeutically effective combination of an anti-cancer immunotherapy agent and a compound according to claim 1.

11. A method according to claim 10, wherein the anti-cancer immunotherapy agent is an antagonistic anti-CTLA-4, anti-PD-1, or anti-PD-L1 antibody.

12. A method according to claim 11, wherein the cancer is lung cancer (including non-small cell lung cancer), pancreatic cancer, kidney cancer, head and neck cancer, lymphoma (including Hodgkin's lymphoma), skin cancer (including melanoma and Merkel skin cancer), urothelial cancer (including bladder cancer), gastric cancer, hepatocellular cancer, or colorectal cancer.

13. A method according to claim 12, wherein the anti-cancer immunotherapy agent is ipilimumab, nivolumab, or pembrolizumab.

14. A compound with a structure according to formula (I′) or (II′) 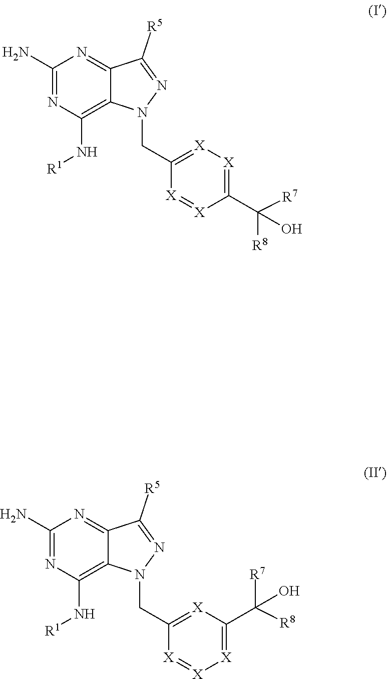
wherein
each X is independently N or CR²;
R¹ is (C₁-C₅ alkyl),
(C₂-C₅ alkenyl),
(C₁-C₈ alkanediyl)₀₋₁(C₃-C₆ cycloalkyl),
(C₁-C₈ alkanediyl)₀₋₁(C₅-C₁₀ spiroalkyl),
(C₂-C₈ alkanediyl)OH,
(C₂-C₈ alkanediyl)O(C₁-C₃ alkyl),
(C₁-C₄ alkanediyl)₀₋₁(5-6 membered heteroaryl),
(C₁-C₄ alkanediyl)₀₋₁phenyl,
(C₁-C₄ alkanediyl)CF₃,
(C₂-C₈ alkanediyl)N[C(═O)](C₁-C₃ alkyl),
or
(C₂-C₈ alkanediyl)NR^{x}R^{y};
each R² is independently H, O(C₁-C₃ alkyl), S(C₁-C₃ alkyl), SO₂(C₁-C₃ alkyl), C₁-C₃ alkyl, O(C₃-C₄ cycloalkyl), S(C₃-C₄ cycloalkyl), SO₂(C₃-C₄ cycloalkyl), C₃-C₄ cycloalkyl, Cl, F, CN; or [C(═O)]₀₋₁NR^{x}R^{y};
R⁵ is H, C₁-C₅ alkyl, C₂-C₅₈ alkenyl, C₃-C₆ cycloalkyl, halo, O(C₁-C₅ alkyl), (C₁-C₄ alkanediyl)OH, (C₁-C₄ alkanediyl)O(C₁-C₃ alkyl), phenyl, NH(C₁-C₅ alkyl), 5 or 6 membered heteroaryl, 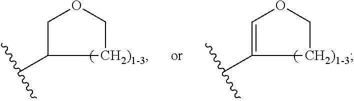
R⁷ and R⁸ are independently
C₁-C₄ alkyl,
C₂-C₄ alkylene,
C₃-C₄ cycloalkyl,
or R⁷ and R⁸ combine with the carbon to which they are bonded to form a 3- to 7-membered cycloalkyl moiety;
R^{x} and R^{y} are independently H or C₁-C₃ alkyl or R^{x} and R^{y} combine with the nitrogen to which they are bonded to form a 3- to 7-membered heterocycle;
wherein in R¹, R², R⁵, R⁷, and R⁸
an alkyl moiety, alkanediyl moiety, cycloalkyl moiety, or a moiety of the formula 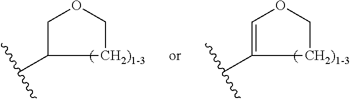
is optionally substituted with one or more substituents selected from OH, halo, CN, (C₁-C₃ alkyl), O(C₁-C₃ alkyl), C(═O)(C₁-C₃ alkyl), SO₂(C₁-C₃ alkyl), NR^{x}R^{y}, (C₁-C₄ alkanediyl)OH, (C₁-C₄ alkanediyl)O(C₁-C₃ alkyl);
and
an alkyl, alkanediyl, cycloalkyl, or moiety of the formula 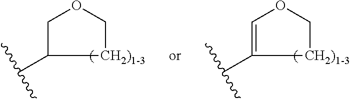
may have a CH₂ group replaced by O, SO₂, CF₂, C(═O), NH,
N[C(═O)]₀₋₁(C₁-C₃ alkyl),
N[C(═O)]₀₋₁(C₁-C₄ alkanediyl)CF₃,
N[C(═O)]₀₋₁(C₁-C₄ alkanediyl)OH,
or
N[C(═O)]₀₋₁(C₁-C₄ alkanediyl)₀₋₁(C₃-C₅ cycloalkyl).

15. A compound having a structure according to formula (Ia′) 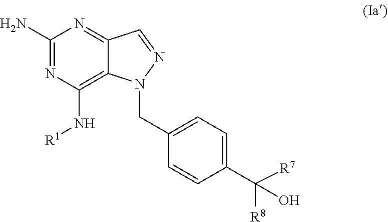
wherein
R¹ is 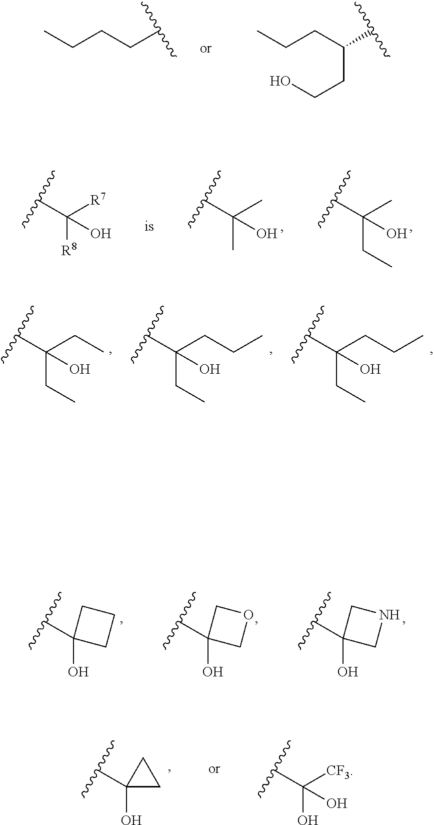
and

16. A compound according to formula (I) of claim 1, wherein the moiety

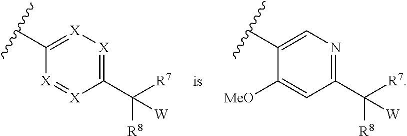

17. A compound according to formula (II) of claim 1, wherein the moiety

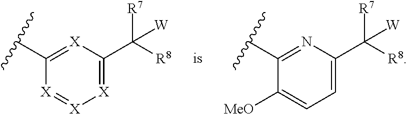

18. A method of treating a cancer, comprising administering to a patient suffering from such cancer a therapeutically effective combination of an anti-cancer immunotherapy agent and a compound according to claim 9.

19. A method according to claim 18, wherein the anti-cancer immunotherapy agent is an antagonistic anti-CTLA-4, anti-PD-1, or anti-PD-L1 antibody.

20. A method according to claim 18, wherein the cancer is lung cancer (including non-small cell lung cancer), pancreatic cancer, kidney cancer, head and neck cancer, lymphoma (including Hodgkin's lymphoma), skin cancer (including melanoma and Merkel skin cancer), urothelial cancer (including bladder cancer), gastric cancer, hepatocellular cancer, or colorectal cancer.

21. A method according to claim 19, wherein the anti-cancer immunotherapy agent is ipilimumab, nivolumab, or pembrolizumab.
